SEQUENCE LISTING (114 sequences)

SEQ ID NO: 1 - protein, 106 aa, Artificial Sequence
  source: /note=“Description of Artificial Sequence Synthetic polypeptide”
EIVLTQSPATMPASPGEKVTLTCRASSSIRYIYWYQQKSGTSPKLWIYDTSKLASGVPNRFSGSGSGTSYSLTISSMETEDTATYYCQQGRSYPLTFGSGTKLEIK

SEQ ID NO: 2 - protein, 116 aa, Artificial Sequence
  source: /note=“Description of Artificial Sequence Synthetic polypeptide”
EVQLVESGGGLVLPGRSMKLSCAASGFTFRTYYMAWVRQAPTKGLEWVASISIGGDNTYYRDSVMGRFTISRDDAKSTLHLQMDNLRSEDTATYYCVRLRGYFDYWGQGVMVTVSS

SEQ ID NO: 3 - protein, 10 aa, Artificial Sequence
  source: /note=“Description of Artificial Sequence Synthetic peptide”
RASSSIRYIY

SEQ ID NO: 4 - protein, 7 aa, Artificial Sequence
  source: /note=“Description of Artificial Sequence Synthetic peptide”
DTSKLAS

SEQ ID NO: 5 - protein, 9 aa, Artificial Sequence
  source: /note=“Description of Artificial Sequence Synthetic peptide”
QQGRSYPLT

SEQ ID NO: 6 - protein, 10 aa, Artificial Sequence
  source: /note=“Description of Artificial Sequence Synthetic peptide”
GFTFRTYYMA

SEQ ID NO: 7 - protein, 17 aa, Artificial Sequence
  source: /note=“Description of Artificial Sequence Synthetic peptide”
SISIGGDNTYYRDSVMG

SEQ ID NO: 8 - protein, 9 aa, Artificial Sequence
  source: /note=“Description of Artificial Sequence Synthetic peptide”
VRLRGYFDY

SEQ ID NO: 9 - protein, 23 aa, Artificial Sequence
  source: /note=“Description of Artificial Sequence Synthetic peptide”
EIVLTQSPATMPASPGEKVTLTC

SEQ ID NO: 10 - protein, 15 aa, Artificial Sequence
  source: /note=“Description of Artificial Sequence Synthetic peptide”
WYQQKSGTSPKLWIY

SEQ ID NO: 11 - protein, 32 aa, Artificial Sequence
  source: /note=“Description of Artificial Sequence Synthetic polypeptide”
GVPNRFSGSGSGTSYSLTISSMETEDTATYYC

SEQ ID NO: 12 - protein, 10 aa, Artificial Sequence
  source: /note=“Description of Artificial Sequence Synthetic peptide”
FGSGTKLEIK

SEQ ID NO: 13 - protein, 25 aa, Artificial Sequence
  source: /note=“Description of Artificial Sequence Synthetic peptide”
EVQLVESGGGLVLPGRSMKLSCAAS

SEQ ID NO: 14 - protein, 14 aa, Artificial Sequence
  source: /note=“Description of Artificial Sequence Synthetic peptide”
WVRQAPTKGLEWVA

SEQ ID NO: 15 - protein, 30 aa, Artificial Sequence
  source: /note=“Description of Artificial Sequence Synthetic polypeptide”
RFTISRDDAKSTLHLQMDNLRSEDTATYYC

SEQ ID NO: 16 - protein, 11 aa, Artificial Sequence
  source: /note=“Description of Artificial Sequence Synthetic peptide”
WGQGVMVTVSS

SEQ ID NO: 17 - protein, 106 aa, Artificial Sequence
  source: /note=“Description of Artificial Sequence Synthetic polypeptide”
NTVMTQSPTSMFISVGDRVTMNCKASRSVGTNVDWYQQKTGQSPTLLFYGASNRYIGVPDRFTGSGSGTDFTLTISNMQAEDLAVYYCLQYNYNWAFGGGTKLELK

SEQ ID NO: 18 - protein, 123 aa, Artificial Sequence
  source: /note=“Description of Artificial Sequence Synthetic polypeptide”
EVQLVESGGGLVQPGRSLKLSCVASGFTFNNYWMTWIRQAPGKGLEWFASITNTGGSTYYPDSVKGRFTISRDNAQSTLYLQTNSLRPEDTATYYCARRDGSYYPYYWYFDLWGPGTTVTVSS

SEQ ID NO: 19 - protein, 11 aa, Artificial Sequence
  source: /note=“Description of Artificial Sequence Synthetic peptide”
KASRSVGTNVD

SEQ ID NO: 20 - protein, 7 aa, Artificial Sequence
  source: /note=“Description of Artificial Sequence Synthetic peptide”
GASNRYI

SEQ ID NO: 21 - protein, 8 aa, Artificial Sequence
  source: /note=“Description of Artificial Sequence Synthetic peptide”
LQYNYNWA

SEQ ID NO: 22 - protein, 10 aa, Artificial Sequence
  source: /note=“Description of Artificial Sequence Synthetic peptide”
GFTFNNYWMT

SEQ ID NO: 23 - protein, 17 aa, Artificial Sequence
  source: /note=“Description of Artificial Sequence Synthetic peptide”
SITNTGGSTYYPDSVKG

SEQ ID NO: 24 - protein, 16 aa, Artificial Sequence
  source: /note=“Description of Artificial Sequence Synthetic peptide”
ARRDGSYYPYYWYFDL

SEQ ID NO: 25 - protein, 23 aa, Artificial Sequence
  source: /note=“Description of Artificial Sequence Synthetic peptide”
NTVMTQSPTSMFISVGDRVTMNC

SEQ ID NO: 26 - protein, 15 aa, Artificial Sequence
  source: /note=“Description of Artificial Sequence Synthetic peptide”
WYQQKTGQSPTLLFY

SEQ ID NO: 27 - protein, 32 aa, Artificial Sequence
  source: /note=“Description of Artificial Sequence Synthetic polypeptide”
GVPDRFTGSGSGTDFTLTISNMQAEDLAVYYC

SEQ ID NO: 28 - protein, 10 aa, Artificial Sequence
  source: /note=“Description of Artificial Sequence Synthetic peptide”
FGGGTKLELK

SEQ ID NO: 29 - protein, 25 aa, Artificial Sequence
  source: /note=“Description of Artificial Sequence Synthetic peptide”
EVQLVESGGGLVQPGRSLKLSCVAS

SEQ ID NO: 30 - protein, 14 aa, Artificial Sequence
  source: /note=“Description of Artificial Sequence Synthetic peptide”
WIRQAPGKGLEWFA

SEQ ID NO: 31 - protein, 30 aa, Artificial Sequence
  source: /note=“Description of Artificial Sequence Synthetic polypeptide”
RFTISRDNAQSTLYLQTNSLRPEDTATYYC

SEQ ID NO: 32 - protein, 11 aa, Artificial Sequence
  source: /note=“Description of Artificial Sequence Synthetic peptide”
WGPGTTVTVSS

SEQ ID NO: 33 - protein, 107 aa, Artificial Sequence
  source: /note=“Description of Artificial Sequence Synthetic polypeptide”
DIQMTQSPASLSASLGETVSIECLASEGISNSLAWYQQKPGKSPQLLIYGASSLQDGVPSRFSGSGSGTQFSLKISGMQPEDEGIYYCQQGYKYPFTFGSGTKLEIK

SEQ ID NO: 34 - protein, 124 aa, Artificial Sequence
  source: /note=“Description of Artificial Sequence Synthetic polypeptide”
EVQLVESGGSLVQPGRSMKVSCAASGFTFTKYYMAWVRQAPTKGLEWVASISTGGGNTYYRDSVKGRFTISRDNAKSTLYLQMDSLRSEDTATYYCARTLINYSDYADYVMDAWGQGASVTVSS

SEQ ID NO: 35 - protein, 11 aa, Artificial Sequence
  source: /note=“Description of Artificial Sequence Synthetic peptide”
LASEGISNSLA

SEQ ID NO: 36 - protein, 7 aa, Artificial Sequence
  source: /note=“Description of Artificial Sequence Synthetic peptide”
GASSLQD

SEQ ID NO: 37 - protein, 9 aa, Artificial Sequence
  source: /note=“Description of Artificial Sequence Synthetic peptide”
QQGYKYPFT

SEQ ID NO: 38 - protein, 10 aa, Artificial Sequence
  source: /note=“Description of Artificial Sequence Synthetic peptide”
GFTFTKYYMA

SEQ ID NO: 39 - protein, 17 aa, Artificial Sequence
  source: /note=“Description of Artificial Sequence Synthetic peptide”
SISTGGGNTYYRDSVKG

SEQ ID NO: 40 - protein, 17 aa, Artificial Sequence
  source: /note=“Description of Artificial Sequence Synthetic peptide”
ARTLINYSDYADYVMDA

SEQ ID NO: 41 - protein, 23 aa, Artificial Sequence
  source: /note=“Description of Artificial Sequence Synthetic peptide”
DIQMTQSPASLSASLGETVSIEC

SEQ ID NO: 42 - protein, 15 aa, Artificial Sequence
  source: /note=“Description of Artificial Sequence Synthetic peptide”
WYQQKPGKSPQLLIY

SEQ ID NO: 43 - protein, 32 aa, Artificial Sequence
  source: /note=“Description of Artificial Sequence Synthetic polypeptide”
GVPSRFSGSGSGTQFSLKISGMQPEDEGIYYC

SEQ ID NO: 44 - protein, 10 aa, Artificial Sequence
  source: /note=“Description of Artificial Sequence Synthetic peptide”
FGSGTKLEIK

SEQ ID NO: 45 - protein, 25 aa, Artificial Sequence
  source: /note=“Description of Artificial Sequence Synthetic peptide”
EVQLVESGGSLVQPGRSMKVSCAAS

SEQ ID NO: 46 - protein, 14 aa, Artificial Sequence
  source: /note=“Description of Artificial Sequence Synthetic peptide”
WVRQAPTKGLEWVA

SEQ ID NO: 47 - protein, 30 aa, Artificial Sequence
  source: /note=“Description of Artificial Sequence Synthetic polypeptide”
RFTISRDNAKSTLYLQMDSLRSEDTATYYC

SEQ ID NO: 48 - protein, 11 aa, Artificial Sequence
  source: /note=“Description of Artificial Sequence Synthetic peptide”
WGQGASVTVSS

SEQ ID NO: 49 - protein, 107 aa, Artificial Sequence
  source: /note=“Description of Artificial Sequence Synthetic polypeptide”
DIQMTQSPASLSASLGETVSIECLASEGISNSLAWYQQKPGKSPQLLIYDASSLQVGVPSRFSGSGSGTQYSLKISGLQPEDEGVYYCQQGYKYPFTFGSGTKLEIK

SEQ ID NO: 50 - protein, 124 aa, Artificial Sequence
  source: /note=“Description of Artificial Sequence Synthetic polypeptide”
EVQLVESGGGLVQPGRSMKLSCAASGFTLSNYYMAWVRQAPTKGLEWVASISTGGGYTYYRDSVKGRFTISRDLAKSTLYLQMDSLRSEDTATYHCARSLINYRNYGDYVMDAWGQGASVTVSS

SEQ ID NO: 51 - protein, 11 aa, Artificial Sequence
  source: /note=“Description of Artificial Sequence Synthetic peptide”
LASEGISNSLA

SEQ ID NO: 52 - protein, 7 aa, Artificial Sequence
  source: /note=“Description of Artificial Sequence Synthetic peptide”
DASSLQV

SEQ ID NO: 53 - protein, 9 aa, Artificial Sequence
  source: /note=“Description of Artificial Sequence Synthetic peptide”
QQGYKYPFT

SEQ ID NO: 54 - protein, 9 aa, Artificial Sequence
  source: /note=“Description of Artificial Sequence Synthetic peptide”
GFTLSNYYM

SEQ ID NO: 55 - protein, 17 aa, Artificial Sequence
  source: /note=“Description of Artificial Sequence Synthetic peptide”
SISTGGGYTYYRDSVKG

SEQ ID NO: 56 - protein, 17 aa, Artificial Sequence
  source: /note=“Description of Artificial Sequence Synthetic peptide”
ARSLINYRNYGDYVMDA

SEQ ID NO: 57 - protein, 23 aa, Artificial Sequence
  source: /note=“Description of Artificial Sequence Synthetic peptide”
DIQMTQSPASLSASLGETVSIEC

SEQ ID NO: 58 - protein, 15 aa, Artificial Sequence
  source: /note=“Description of Artificial Sequence Synthetic peptide”
WYQQKPGKSPQLLIY

SEQ ID NO: 59 - protein, 32 aa, Artificial Sequence
  source: /note=“Description of Artificial Sequence Synthetic polypeptide”
GVPSRFSGSGSGTQYSLKISGLQPEDEGVYYC

SEQ ID NO: 60 - protein, 10 aa, Artificial Sequence
  source: /note=“Description of Artificial Sequence Synthetic peptide”
FGSGTKLEIK

SEQ ID NO: 61 - protein, 25 aa, Artificial Sequence
  source: /note=“Description of Artificial Sequence Synthetic peptide”
EVQLVESGGGLVQPGRSMKLSCAAS

SEQ ID NO: 62 - protein, 14 aa, Artificial Sequence
  source: /note=“Description of Artificial Sequence Synthetic peptide”
WVRQAPTKGLEWVA

SEQ ID NO: 63 - protein, 30 aa, Artificial Sequence
  source: /note=“Description of Artificial Sequence Synthetic polypeptide”
RFTISRDLAKSTLYLQMDSLRSEDTATYHC

SEQ ID NO: 64 - protein, 11 aa, Artificial Sequence
  source: /note=“Description of Artificial Sequence Synthetic peptide”
WGQGASVTVSS

SEQ ID NO: 65 - protein, 111 aa, Artificial Sequence
  source: /note=“Description of Artificial Sequence Synthetic polypeptide”
QFTLTQPKSVSGSLRSTITIPCERSSGDIGHNYVSWYQQHLGRPPINVIYADDQRPSEVSDRFSGSIDSSSNSASLTITNLQMDDEADYFCQSYDSNVDIVFGGGTKLTVL

SEQ ID NO: 66 - protein, 120 aa, Artificial Sequence
  source: /note=“Description of Artificial Sequence Synthetic polypeptide”
QVQLKESGPGLVQPSQTLSLTCSVSGLSLTTSSLSWIRQPPGKGLEWMGGIWSKGGTEYNSPIKSRLSISRDTSKSQIFLKMNSLQTEDTAMYFCARNGVYHNYWYFDFWGPGTMVTVSS

SEQ ID NO: 67 - protein, 13 aa, Artificial Sequence
  source: /note=“Description of Artificial Sequence Synthetic peptide”
ERSSGDIGHNYVS

SEQ ID NO: 68 - protein, 7 aa, Artificial Sequence
  source: /note=“Description of Artificial Sequence Synthetic peptide”
ADDQRPS

SEQ ID NO: 69 - protein, 10 aa, Artificial Sequence
  source: /note=“Description of Artificial Sequence Synthetic peptide”
QSYDSNVDIV

SEQ ID NO: 70 - protein, 10 aa, Artificial Sequence
  source: /note=“Description of Artificial Sequence Synthetic peptide”
GLSLTTSSLS

SEQ ID NO: 71 - protein, 16 aa, Artificial Sequence
  source: /note=“Description of Artificial Sequence Synthetic peptide”
GIWSKGGTEYNSPIKS

SEQ ID NO: 72 - protein, 14 aa, Artificial Sequence
  source: /note=“Description of Artificial Sequence Synthetic peptide”
ARNGVYHNYWYFDF

SEQ ID NO: 73 - protein, 22 aa, Artificial Sequence
  source: /note=“Description of Artificial Sequence Synthetic peptide”
QFTLTQPKSVSGSLRSTITIPC

SEQ ID NO: 74 - protein, 15 aa, Artificial Sequence
  source: /note=“Description of Artificial Sequence Synthetic peptide”
WYQQHLGRPPINVIY

SEQ ID NO: 75 - protein, 34 aa, Artificial Sequence
  source: /note=“Description of Artificial Sequence Synthetic polypeptide”
EVSDRFSGSIDSSSNSASLTITNLQMDDEADYFC

SEQ ID NO: 76 - protein, 10 aa, Artificial Sequence
  source: /note=“Description of Artificial Sequence Synthetic peptide”
FGGGTKLTVL

SEQ ID NO: 77 - protein, 25 aa, Artificial Sequence
  source: /note=“Description of Artificial Sequence Synthetic peptide”
QVQLKESGPGLVQPSQTLSLTCSVS

SEQ ID NO: 78 - protein, 14 aa, Artificial Sequence
  source: /note=“Description of Artificial Sequence Synthetic peptide”
WIRQPPGKGLEWMG

SEQ ID NO: 79 - protein, 30 aa, Artificial Sequence
  source: /note=“Description of Artificial Sequence Synthetic polypeptide”
RLSISRDTSKSQIFLKMNSLQTEDTAMYFC

SEQ ID NO: 80 - protein, 11 aa, Artificial Sequence
  source: /note=“Description of Artificial Sequence Synthetic peptide”
WGPGTMVTVSS

SEQ ID NO: 81 - protein, 107 aa, Artificial Sequence
  source: /note=“Description of Artificial Sequence Synthetic polypeptide”
DIVLTQSPSSLSASLGDTITITCHASQNINVWLSWYQQKPGNIPKLLIYKASNLHSGVPSRFSGSGSGTGFTLTISSLQPEDIATYYCQQGQTFPYTFGGGTRLEIK

SEQ ID NO: 82 - protein, 122 aa, Artificial Sequence
  source: /note=“Description of Artificial Sequence Synthetic polypeptide”
QVQLQQPGSVLVRPGASVKLSCKASGYTFTGSWLHWAKQRPGQGLEWIGEIHPYSGNTNYNERFKGKATLTVDTPSSTAYVDLRSLTFEDSAVYYCAKEGGWFLRIYGMDYWGQGTSVTVSS

SEQ ID NO: 83 - protein, 11 aa, Artificial Sequence
  source: /note=“Description of Artificial Sequence Synthetic peptide”
HASQNINVWLS

SEQ ID NO: 84 - protein, 7 aa, Artificial Sequence
  source: /note=“Description of Artificial Sequence Synthetic peptide”
KASNLHS

SEQ ID NO: 85 - protein, 9 aa, Artificial Sequence
  source: /note=“Description of Artificial Sequence Synthetic peptide”
QQGQTFPYT

SEQ ID NO: 86 - protein, 10 aa, Artificial Sequence
  source: /note=“Description of Artificial Sequence Synthetic peptide”
GYTFTGSWLH

SEQ ID NO: 87 - protein, 17 aa, Artificial Sequence
  source: /note=“Description of Artificial Sequence Synthetic peptide”
EIHPYSGNTNYNERFKG

SEQ ID NO: 88 - protein, 15 aa, Artificial Sequence
  source: /note=“Description of Artificial Sequence Synthetic peptide”
AKEGGWFLRIYGMDY

SEQ ID NO: 89 - protein, 23 aa, Artificial Sequence
  source: /note=“Description of Artificial Sequence Synthetic peptide”
DIVLTQSPSSLSASLGDTITITC

SEQ ID NO: 90 - protein, 15 aa, Artificial Sequence
  source: /note=“Description of Artificial Sequence Synthetic peptide”
WYQQKPGNIPKLLIY

SEQ ID NO: 91 - protein, 32 aa, Artificial Sequence
  source: /note=“Description of Artificial Sequence Synthetic polypeptide”
GVPSRFSGSGSGTGFTLTISSLQPEDIATYYC

SEQ ID NO: 92 - protein, 10 aa, Artificial Sequence
  source: /note=“Description of Artificial Sequence Synthetic peptide”
FGGGTRLEIK

SEQ ID NO: 93 - protein, 25 aa, Artificial Sequence
  source: /note=“Description of Artificial Sequence Synthetic peptide”
QVQLQQPGSVLVRPGASVKLSCKAS

SEQ ID NO: 94 - protein, 14 aa, Artificial Sequence
  source: /note=“Description of Artificial Sequence Synthetic peptide”
WAKQRPGQGLEWIG

SEQ ID NO: 95 - protein, 30 aa, Artificial Sequence
  source: /note=“Description of Artificial Sequence Synthetic polypeptide”
KATLTVDTPSSTAYVDLRSLTFEDSAVYYC

SEQ ID NO: 96 - protein, 11 aa, Artificial Sequence
  source: /note=“Description of Artificial Sequence Synthetic peptide”
WGQGTSVTVSS

SEQ ID NO: 97 - protein, 621 aa, Homo sapiens
MELQPPEASIAVVSIPRQLPGSHSEAGVQGLSAGDDSETGSDCVTQAGLQLLASSDPPALASKNAEVTVETGFHHVSQADIEFLTSIDPTASASGSAGITGTMSQDTEVDMKEVELNELEPEKQPMNAASGAAMSLAGAEKNGLVKIKVAEDEAEAAAAAKFTGLSKEELLKVAGSPGWVWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 98 - DNA, 2350 bp, Homo sapiens
agttccagggaaggagggcgtagacaaagcgccacctgaacttgcggcgcgaaaaaggcgcgcatgcgtcctacgggagcgtgctggctcaccgaccgcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagacggggtctgactgtgttacccaggctggtcttcaactcttggcctcaagtgatcctcctgccttagcttccaagaatgctgaggttacagtagaaacggggtttcaccatgttagccaggctgatattgaattcctgacctcaattgatccgactgcctcggcctccggaagtgctgggattacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaagtcttccctctgcttctctcataaaaaaa

SEQ ID NO: 99 - protein, 630 aa, Homo sapiens
MELQPPEASIAVVSIPRQLPGSHSEAGVQGLSAGDDSELGSHCVAQTGLELLASGDPLPSASQNAEMIETGSDCVTQAGLQLLASSDPPALASKNAEVTGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 100 - DNA, 2347 bp, Homo sapiens
agttccagggaaggagggcgtagacaaagcgccacctgaacttgcggcgcgaaaaaggcgcgcatgcgtcctacgggagcgtgctggctcaccgaccgcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcagagttggggtctcactgtgttgcccagactggtctcgaactcttggcctcaggtgatcctcttccctcagcttcccagaatgccgagatgatagagacggggtctgactgtgttacccaggctggtcttcaactcttggcctcaagtgatcctcctgccttagcttccaagaatgctgaggttacaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaagtcttccctctgcttctctcataaaaaaa

SEQ ID NO: 101 - protein, 568 aa, Homo sapiens
MELQPPEASIAVVSIPRQLPGSHSEAGVQGLSAGDDSGTMSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 102 - DNA, 2161 bp, Homo sapiens
agttccagggaaggagggcgtagacaaagcgccacctgaacttgcggcgcgaaaaaggcgcgcatgcgtcctacgggagcgtgctggctcaccgaccgcattgcggcttggttttctcacccagtgcatgtggcaggagcggtgagatcactgcctcacggcgatcctggactgacggtcacgactgcctaccctctaaccctgttctgagctgccccttgcccacacaccccaaacctgtgtgcaggatccgcctccatggagctacagcctcctgaagcctcgatcgccgtcgtgtcgattccgcgccagttgcctggctcacattcggaggctggtgtccagggtctcagcgcgggggacgactcaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaagtcttccctctgcttctctcataaaaaaa

SEQ ID NO: 103 - protein, 529 aa, Homo sapiens
MSQDTEVDMKEVELNELEPEKQPMNAASGAAMSLAGAEKNGLVKIKVAEDEAEAAAAAKFTGLSKEELLKVAGSPGWVRTRWALLLLFWLGWLGMLAGAVVIIVRAPRCRELPAQKWWHTGALYRIGDLQAFQGHGAGNLAGLKGRLDYLSSLKVKGLVLGPIHKNQKDDVAQTDLLQIDPNFGSKEDFDSLLQSAKKKSIRVILDLTPNYRGENSWFSTQVDTVATKVKDALEFWLQAGVDGFQVRDIENLKDASSFLAEWQNITKGFSEDRLLIAGTNSSDLQQILSLLESNKDLLLTSSYLSDSGSTGEHTKSLVTQYLNATGNRWCSWSLSQARLLTSFLPAQLLRLYQLMLFTLPGTPVFSYGDEIGLDAAALPGQPMEAPVMLWDESSFPDIPGAVSANMTVKGQSEDPGSLLSLFRRLSDQRSKERSLLHGDFHAFSAGPGLFSYIRHWDQNERFLVVLNFGDVGLSAGLQASDLPASASLPAKADLLLSTQPGREEGSPLELERLKLEPHEGLLLRFPYAA

SEQ ID NO: 104 - DNA, 1938 bp, Homo sapiens
cagaggccgcgcctgctgctgagcagatgcagtagccgaaactgcgcggaggcacagaggccggggagagcgttctgggtccgagggtccaggtaggggttgagccaccatctgaccgcaagctgcgtcgtgtcgccggttctgcaggcaccatgagccaggacaccgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggcagccgcggctaagttcacgggcctgtccaaggaggagctgctgaaggtggcaggcagccccggctgggtacgcacccgctgggcactgctgctgctcttctggctcggctggctcggcatgcttgctggtgccgtggtcataatcgtgcgagcgccgcgttgtcgcgagctaccggcgcagaagtggtggcacacgggcgccctctaccgcatcggcgaccttcaggccttccagggccacggcgcgggcaacctggcgggtctgaaggggcgtctcgattacctgagctctctgaaggtgaagggccttgtgctgggtccaattcacaagaaccagaaggatgatgtcgctcagactgacttgctgcagatcgaccccaattttggctccaaggaagattttgacagtctcttgcaatcggctaaaaaaaagagcatccgtgtcattctggaccttactcccaactaccggggtgagaactcgtggttctccactcaggttgacactgtggccaccaaggtgaaggatgctctggagttttggctgcaagctggcgtggatgggttccaggttcgggacatagagaatctgaaggatgcatcctcattcttggctgagtggcaaaatatcaccaagggcttcagtgaagacaggctcttgattgcggggactaactcctccgaccttcagcagatcctgagcctactcgaatccaacaaagacttgctgttgactagctcatacctgtctgattctggttctactggggagcatacaaaatccctagtcacacagtatttgaatgccactggcaatcgctggtgcagctggagtttgtctcaggcaaggctcctgacttccttcttgccggctcaacttctccgactctaccagctgatgctcttcaccctgccagggacccctgttttcagctacggggatgagattggcctggatgcagctgcccttcctggacagcctatggaggctccagtcatgctgtgggatgagtccagcttccctgacatcccaggggctgtaagtgccaacatgactgtgaagggccagagtgaagaccctggctccctcctttccttgttccggcggctgagtgaccagcggagtaaggagcgctccctactgcatggggacttccacgcgttctccgctgggcctggactcttctcctatatccgccactgggaccagaatgagcgttttctggtagtgcttaactttggggatgtgggcctctcggctggactgcaggcctccgacctgcctgccagcgccagcctgccagccaaggctgacctcctgctcagcacccagccaggccgtgaggagggctcccctcttgagctggaacgcctgaaactggagcctcacgaagggctgctgctccgcttcccctacgcggcctgacttcagcctgacatggacccactacccttctcctttccttcccaggccctttggcttctgatttttctcttttttaaaaacaaacaaacaaactgttgcagattatgagtgaacccccaaatagggtgttttctgccttcaaataaaagtcacccctgcatggtgaagtcttccctctgcttctctcataaaaaaa

SEQ ID NO: 105 - protein, 565 aa, Mus musculus
MDPEPTEHSTDGVSVPRQPPSAQTGLDVQVVSAAGDSGTMSQDTEVDMKDVELNELEPEKQPMNAADGAAAGEKNGLVKIKVAEDETEAGVKFTGLSKEELLKVAGSPGWVRTRWALLLLFWLGWLGMLAGAVVIIVRAPRCRELPVQRWWHKGALYRIGDLQAFVGRDAGGIAGLKSHLEYLSTLKVKGLVLGPIHKNQKDEINETDLKQINPTLGSQEDFKDLLQSAKKKSIHIILDLTPNYQGQNAWFLPAQADIVATKMKEALSSWLQDGVDGFQFRDVGKLMNAPLYLAEWQNITKNLSEDRLLIAGTESSDLQQIVNILESTSDLLLTSSYLSNSTFTGERTESLVTRFLNATGSQWCSWSVSQAGLLADFIPDHLLRLYQLLLFTLPGTPVFSYGDELGLQGALPGQPAKAPLMPWNESSIFHIPRPVSLNMTVKGQNEDPGSLLTQFRRLSDLRGKERSLLHGDFHALSSSPDLFSYIRHWDQNERYLVVLNFRDSGRSARLGASNLPAGISLPASAKLLLSTDSARQSREEDTSLKLENLSLNPYEGLLLQFPFVA

SEQ ID NO: 106 - DNA, 2813 bp, Mus musculus
gtgggtagaggaatccgcccaaaggggcgtgcggagagctccgcctctgattttgcagcgcgaaaaagaggcgcaggcgctttaggggagtgcgacgctacgcctttggcgctgcggctaggcggttcttactcactgcgggtaaaacgtcatcgctggagattttggttcgcgacccatacagctcgactgtctgggtcacaactaccaatatccatacgttgaggcgatttctcaccctcactcacgctaagccgcgtgttgatccatctctatggatcctgaacctactgaacactccaccgacggtgtctcggttccccgccagccgcccagcgcgcagacggggcttgatgtccaggttgtcagcgcagcgggcgactcaggcaccatgagccaggacaccgaagtggacatgaaagatgtggagctgaacgagctagaaccggagaagcagcccatgaatgcagcggacggggcggcggccggggagaagaacggtctggtgaagatcaaggtggcggaggacgagacggaggccggggtcaagttcaccggcttatccaaggaggagctactgaaggtagcgggcagccctggctgggtgcgcacccgctgggcgctgctgctgctcttctggctcggttggctgggcatgctggcgggcgccgtggttatcatcgttcgggcgccgcgctgccgtgagctgcctgtacagaggtggtggcacaagggcgccctctaccgcatcggcgaccttcaggcctttgtaggccgggatgcgggaggcatagctggtctgaagagccatctggagtacttgagcaccctgaaggtgaagggcctggtgttaggcccaattcacaagaaccagaaggatgaaatcaatgaaaccgacctgaaacagattaatcccactttgggctcccaggaagattttaaagaccttctacaaagtgccaagaaaaagagcattcacatcattttggacctcactcccaactaccagggccagaatgcgtggttcctccctgctcaggctgacattgtagccaccaaaatgaaggaagctctgagttcttggttgcaggacggtgtggatggtttccaattccgggatgtgggaaagctgatgaatgcacccttgtacttggctgagtggcagaatatcaccaagaacttaagtgaggacaggcttttgattgcagggactgagtcctctgacctgcagcaaattgtcaacatacttgaatccaccagcgacctgctgttgaccagctcctacctgtcaaattccactttcactggggagcgtactgaatccctagtcactaggtttttgaatgccactggcagccaatggtgcagctggagtgtgtcgcaagcaggactcctcgcagactttataccggaccatcttctccgactctaccagctgctgctcttcactctgccagggactcctgtttttagctacggggatgagcttggccttcagggtgcccttcctggacagcctgcgaaggccccactcatgccgtggaatgagtccagcatctttcacatcccaagacctgtaagcctcaacatgacagtgaagggccagaatgaagaccctggctccctccttacccagttccggcggctgagtgaccttcggggtaaggagcgctctctgttgcacggtgacttccatgcactgtcttcctcacctgacctcttctcctacatacgacactgggaccagaatgagcgttacctggtggtgctcaacttccgagattcgggccggtcagccaggctaggggcctccaacctccctgctggcataagcctgccagccagcgctaaacttttgcttagtaccgacagtgcccggcaaagccgtgaggaggacacctccctgaagctggaaaacctgagcctgaatccttatgagggcttgctgttacagttcccctttgtggcctgatccttcctatgcagaacctaccaccctcctttgttctccccaggccttttggattctagtcttcctctccttgtttttaaacttttgcagattacatacgaattcttatactgggtgtttttgtcttcaaataaaaacatcacccctgcctcatgagattgtgactttcatccttccttccttctagaagaactttctcttgctcctgatctcttttgctcctccctgcccctgccatagtcgcagccagttgtagacagctattccagctctctttttttttttttttttttttttttttttggtttttcgagacagggtttctctgtatagccctggctgtcctggaactcactttgtagaccaggctggcctcgaactcagaaatccacctgcctctgcctcccaagtgctgggattaaaggcgtgcgccaccacgcccggccgctattccagctcttaaattaatcatttagagaccaaggctagagaagggcccttccatggttaacagcaaagtgtcttggctggagtaaccacacctcctcgctctggcccaagaatcttgggaattgccaactcttccttatctctcttagcacagtctttaagaaaaagggtggggtgagttgaagactgcatactgccaagggcctggggcttcccttctttactctttggtgaggcacttaccatatagacaggactgcgatccccagtacccagtggataccccatctccagaaaaagccaacaagacaaaccctttgcttccttaggctatgttatctcttgtgtggaaatggagaagaaataaggaataaacattttttgtatgaag

SEQ ID NO: 107 - protein, 526 aa, Mus musculus
MSQDTEVDMKDVELNELEPEKQPMNAADGAAAGEKNGLVKIKVAEDETEAGVKFTGLSKEELLKVAGSPGWVRTRWALLLLFWLGWLGMLAGAVVIIVRAPRCRELPVQRWWHKGALYRIGDLQAFVGRDAGGIAGLKSHLEYLSTLKVKGLVLGPIHKNQKDEINETDLKQINPTLGSQEDFKDLLQSAKKKSIHIILDLTPNYQGQNAWFLPAQADIVATKMKEALSSWLQDGVDGFQFRDVGKLMNAPLYLAEWQNITKNLSEDRLLIAGTESSDLQQIVNILESTSDLLLTSSYLSNSTFTGERTESLVTRFLNATGSQWCSWSVSQAGLLADFIPDHLLRLYQLLLFTLPGTPVFSYGDELGLQGALPGQPAKAPLMPWNESSIFHIPRPVSLNMTVKGQNEDPGSLLTQFRRLSDLRGKERSLLHGDFHALSSSPDLFSYIRHWDQNERYLVVLNFRDSGRSARLGASNLPAGISLPASAKLLLSTDSARQSREEDTSLKLENLSLNPYEGLLLQFPFVA

SEQ ID NO: 108 - DNA, 2572 bp, Mus musculus
cccgccgccacacccgcccagcggcagaagcagttaggaagctctgctagcctcacggccacgggacgcctctctgaacggggatccaggcaggattagagctgcctcactgactacaggccgtgtcgtgtcaccgtttctgcaggcaccatgagccaggacaccgaagtggacatgaaagatgtggagctgaacgagctagaaccggagaagcagcccatgaatgcagcggacggggcggcggccggggagaagaacggtctggtgaagatcaaggtggcggaggacgagacggaggccggggtcaagttcaccggcttatccaaggaggagctactgaaggtagcgggcagccctggctgggtgcgcacccgctgggcgctgctgctgctcttctggctcggttggctgggcatgctggcgggcgccgtggttatcatcgttcgggcgccgcgctgccgtgagctgcctgtacagaggtggtggcacaagggcgccctctaccgcatcggcgaccttcaggcctttgtaggccgggatgcgggaggcatagctggtctgaagagccatctggagtacttgagcaccctgaaggtgaagggcctggtgttaggcccaattcacaagaaccagaaggatgaaatcaatgaaaccgacctgaaacagattaatcccactttgggctcccaggaagattttaaagaccttctacaaagtgccaagaaaaagagcattcacatcattttggacctcactcccaactaccagggccagaatgcgtggttcctccctgctcaggctgacattgtagccaccaaaatgaaggaagctctgagttcttggttgcaggacggtgtggatggtttccaattccgggatgtgggaaagctgatgaatgcacccttgtacttggctgagtggcagaatatcaccaagaacttaagtgaggacaggcttttgattgcagggactgagtcctctgacctgcagcaaattgtcaacatacttgaatccaccagcgacctgctgttgaccagctcctacctgtcaaattccactttcactggggagcgtactgaatccctagtcactaggtttttgaatgccactggcagccaatggtgcagctggagtgtgtcgcaagcaggactcctcgcagactttataccggaccatcttctccgactctaccagctgctgctcttcactctgccagggactcctgtttttagctacggggatgagcttggccttcagggtgcccttcctggacagcctgcgaaggccccactcatgccgtggaatgagtccagcatctttcacatcccaagacctgtaagcctcaacatgacagtgaagggccagaatgaagaccctggctccctccttacccagttccggcggctgagtgaccttcggggtaaggagcgctctctgttgcacggtgacttccatgcactgtcttcctcacctgacctcttctcctacatacgacactgggaccagaatgagcgttacctggtggtgctcaacttccgagattcgggccggtcagccaggctaggggcctccaacctccctgctggcataagcctgccagccagcgctaaacttttgcttagtaccgacagtgcccggcaaagccgtgaggaggacacctccctgaagctggaaaacctgagcctgaatccttatgagggcttgctgttacagttcccctttgtggcctgatccttcctatgcagaacctaccaccctcctttgttctccccaggccttttggattctagtcttcctctccttgtttttaaacttttgcagattacatacgaattcttatactgggtgtttttgtcttcaaataaaaacatcacccctgcctcatgagattgtgactttcatccttccttccttctagaagaactttctcttgctcctgatctcttttgctcctccctgcccctgccatagtcgcagccagttgtagacagctattccagctctctttttttttttttttttttttttttttttggtttttcgagacagggtttctctgtatagccctggctgtcctggaactcactttgtagaccaggctggcctcgaactcagaaatccacctgcctctgcctcccaagtgctgggattaaaggcgtgcgccaccacgcccggccgctattccagctcttaaattaatcatttagagaccaaggctagagaagggcccttccatggttaacagcaaagtgtcttggctggagtaaccacacctcctcgctctggcccaagaatcttgggaattgccaactcttccttatctctcttagcacagtctttaagaaaaagggtggggtgagttgaagactgcatactgccaagggcctggggcttcccttctttactctttggtgaggcacttaccatatagacaggactgcgatccccagtacccagtggataccccatctccagaaaaagccaacaagacaaaccctttgcttccttaggctatgttatctcttgtgtggaaatggagaagaaataaggaataaacattttttgtatgaag

SEQ ID NO: 109 - protein, 529 aa, Macaca fascicularis
MSQDTEVDMKEVELNELEPEKQPMNAASGAAMAVVGAEKNGLVKIKVAEDEAEAAAAAKFTGLSKEELLKVAGSPGWVRTRWVLLLLFWLGWLGMLAGAVVIIVRAPRCRELPAQKWWHTGALYRIGDLQAFQGHGSGNLAGLKGRLDYLSSLKVKGLVLGPLHKNQKDDVAQTDLLQIDPNFGSKEDFDNLLQSAKKKSIRVILDLTPNYRGENLWFSTQVDSVATKVKDALEFWLQAGVDGFQVRDIENLKDASSFLAEWENITKGFSEDRLLIAGTNSSDLQQIVSPLESNKDLLLTSSYLSDSSFTGEHTKSLVTQYLNATGNRWCSWSLSQAGLLTSFLPAQLLRLYQLMLSTLPGTPVFSYGDEIGLKAAALPGQPVEAPVMLWDESSFPDIPGAVSANMTVKGQSEDPGSLLSLFRQLSDQRSKERSLLHGDFHTFSSGPGLFSYIRHWDQNERFLVVLNFGDVGLSAGLQASDLPASASLPTKADPVLSTQPGREEGSPLELERLKLEPHEGLLLRFPYVA

SEQ ID NO: 110 - DNA, 1922 bp, Macaca fascicularis
agatgcagtagccgaagctgcgcggaggcacacaggccgggagaccgttctgggtccgagggtccgggcaggggttgagccaccatctgacctcaagcttcgtcgtgtcgccggttctgcaggcaccatgagccaggacaccgaggtggatatgaaggaggtggagctgaatgagttagaacccgagaagcagccgatgaacgcggcgtctggggctgccatggccgtggtgggagccgagaagaatggtctggtgaagatcaaggtggcggaagacgaggcggaggcagcagccgccgctaagttcacgggcctgtccaaggaggagctgctgaaggtggcgggcagtcccggctgggtacgtacccgctgggtgctgctgctgctcttctggctcggctggcttggcatgctggcgggtgccgtggtcataatcgtgcgggcgccgcgctgtcgcgagctgccggcgcagaagtggtggcacacgggcgccctctaccgcatcggcgaccttcaggccttccagggccacggctcgggcaacttggcgggtctgaaggggcgtctcgattacctgagctctctgaaggtgaagggccttgtgctgggcccacttcacaagaaccagaaggacgatgtcgctcagaccgacttgctgcagatcgaccccaattttggctccaaggaagattttgacaatctcttgcaatcggctaaaaaaaagagcatccgtgtcattctggacctcactcccaactaccggggtgagaacttgtggttctccacccaggttgacagtgtggccaccaaggtgaaggatgctctggagttttggctgcaagctggcgtggatgggttccaggttcgggacatagagaatctgaaggatgcatcctcattcttggctgagtgggaaaacatcaccaagggcttcagtgaagataggctcttgattgcagggactaactcctccgaccttcagcagatcgtgagcccactcgaatccaacaaagacttgctgttgaccagctcatacctgtctgattccagctttactggggagcatacaaaatccctagtcacacagtatttgaatgccactggcaatcgctggtgcagctggagtttgtctcaggcagggctcctgacttccttcttgccggctcaacttctccgactctaccagctgatgctctccaccctgccagggacccctgtgttcagctacggggatgagattggcctgaaggcagctgcccttcctggacagcctgtggaggctccagtcatgctgtgggatgagtccagcttccctgacatcccaggggctgtaagtgccaacatgactgtgaagggccagagtgaagaccctggctccctcctttccttgttccggcagctgagtgaccagcggagtaaggagcgctccctattgcatggggacttccatacgttctcctctgggcctggactcttctcctatatccgccactgggaccagaatgagcgttttctggtagtgcttaactttggggatgtgggcctctcggctgggctgcaggcctccgacctgcccgccagcgccagcctgccaaccaaggctgaccctgtgctcagcacccagccaggccgtgaggagggctccccgcttgagctggaacgcctgaaactggagcctcacgaagggctgctgctccgcttcccctatgtggcctgaccccagcctgacgtggacccactgccctcctttccttcctagaccctttgggttctggtttttctctttttcccccttttttaaaaaacaacaacaaaacggttgcagattataaatgaacccccaaatagggtgttttctgccttcaaataaaagtcacccctgcctggtgaaaaaaaaaaaaaaaaaaaaaaaaaaaaaaaaa

SEQ ID NO: 111 - protein, 501 aa, Homo sapiens [sequence omitted]

SEQ ID NO: 112 - protein, 255 aa, Homo sapiens
MAAALFVLLGFALLGTHGASGAAGTVFTTVEDLGSKILLTCSLNDSATEVTGHRWLKGGVVLKEDALPGQKTEFKVDSDDQWGEYSCVFLPEPMGTANIQLHGPPRVKAVKSSEHINEGETAMLVCKSESVPPVTDWAWYKITDSEDKALMNGSESRFFVSSSQGRSELHIENLNMEADPGQYRCNGTSSKGSDQAIITLRVRSVLVLVTIIFIYEKRRKPEDVLDDDDAGSAPLKSSGQHQNDKGKNVRQRNSS

SEQ ID NO: 113 - protein, 454 aa, Mus musculus
MAAALLLALAFTLLSGQGACAAAGTIQTSVQEVNSKTQLTCSLNSSGVDIVGHRWMRGGKVLQEDTLPDLHTKYIVDADDRSGEYSCIFLPEPVGRSEINVEGPPRIKVGKKSEHSSEGELAKLVCKSDASYPPITDWFWFKTSDTGEEEAITNSTEANGKYVVVSTPEKSQLTISNLDVNVDPGTYVCNATNAQGTTRETISLRVRSRGNSRAQVTDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 114 - protein, 492 aa, Homo sapiens
MEPSSKKLTGRLMLAVGGAVLGSLQFGYNTGVINAPQKVIEEFYNQTWVHRYGESILPTTLTTLWSLSVAIFSVGGMIGSFSVGLFVNRFGRRNSMLMMNLLAFVSAVLMGFSKLGKSFEMLILGRFIIGVYCGLTTGFVPMYVGEVSPTALRGALGTLHQLGIVVGILIAQVFGLDSIMGNKDLWPLLLSIIFIPALLQCIVLPFCPESPRFLLINRNEENRAKSVLKKLRGTADVTHDLQEMKEESRQMMREKKVTILELFRSPAYRQPILIAVVLQLSQQLSGINAVFYYSTSIFEKAGVQQPVYATIGSGIVNTAFTVVSLFVVERAGRRTLHLIGLAGMAGCAILMTIALALLEQLPWMSYLSIVAIFGFVAFFEVGPGPIPWFIVAELFSQGPRPAAIAVAGFSNWTSNFIVGMCFQYVEQLCGPYVFIIFTVLLVLFFIFTYFKVPETKGRTFDEIASGFRQGGASQSDKTPEELFHPLGADSQV

(19) United States
(12) Patent Application Publication
(10) Pub. No.: US 2018/0000964 A1
(43) Pub. Date: Jan. 4, 2018

(54) BLOOD BRAIN BARRIER RECEPTOR ANTIBODIES AND METHODS OF USE

(71) Applicant: Genentech, Inc., South San Francisco, CA (US)

(72) Inventors: Christine Tan, San Mateo, CA (US); Ryan J. Watts, San Mateo, CA (US); Joy Yu Zuchero, San Mateo, CA (US); Xiaocheng Chen, Foster City, CA (US); Mark S. Dennis, San Carlos, CA (US)

(73) Assignee: Genentech, Inc., South San Francisco, CA (US)

(21) Appl. No.: 15/618,467

(22) Filed: Jun. 9, 2017

Related U.S. Application Data
(63) Continuation of application No. PCT/US2015/064805, filed on Dec. 9, 2015.
(60) Provisional application No. 62/251,983, filed on Nov. 6, 2015.
(60) Provisional application No. 62/090,295, filed on Dec. 10, 2014.

(51) Int. Cl. A61K 47/68; C07K 16/40; C07K 16/18; C07K 16/28

(52) U.S. Cl. CPC A61K 47/6849; C07K 16/2803; C07K 16/2896; C07K 16/40; C12Y 304/23046; C07K 16/18; C07K 2317/31; C07K 2317/565; C07K 2317/567; C07K 2317/92; C07K 2317/55

(57) ABSTRACT

The present invention relates to antibodies that bind to receptors expressed on the blood brain barrier and methods of using the same.

### CROSS REFERENCE TO RELATED APPLICATIONS

This application is a continuation of International Application Number PCT/US2015/064805, filed Dec. 9, 2015, the entire contents of which are incorporated herein by reference, and which claims the benefit under 35 USC §119(e) of U.S. Provisional Application No. 62/090,295, filed on Dec. 10, 2014, and Provisional Application No. 62/251,983, filed on Nov. 6, 2015.

### FIELD OF THE INVENTION

The present invention relates to antibodies that bind to receptors expressed on the blood brain barrier and methods of using the same.

### BACKGROUND

Brain penetration of large molecule drugs is severely limited by the largely impermeable blood-brain barrier (BBB). One strategy to overcome this obstacle is to utilize transcytosis trafficking pathways of endogenous receptors expressed at the brain capillary endothelium. Recombinant proteins such as monoclonal antibodies have been designed against these receptors to enable receptor-mediated delivery of large molecules to the brain.

Since these receptors carry out important biological functions, such as transports of essential amino acids, glucose, and other resources needed in the brain, it is important that the transport of those molecules is not blocked by such targeting antibodies. Further, since the receptors expressed on the BBB are often also expressed in other compartments, it is also important that such antibodies do not have dangerous, off-target effects.

### SUMMARY

Receptor mediated transport (RMT)-based bispecific targeting technology has the potential to open the door for a wide range of potential therapeutics for CNS diseases. Past studies have shown that antibodies against the transferrin receptor can deliver therapeutics including antibodies and small molecules across the BBB at both trace and therapeutically relevant doses after a single systemic injection in mice (see, e.g., WO 2012/075037). As discussed above, important considerations when designing these technologies include preservation of the transport function of target BBB receptors (BBB-Rs), and the safety profile. The present disclosure provides new targets for the RMT-based targeting technology, as well as antibodies specific for those targets.

For example, as demonstrated in the Examples below, novel BBB-R targets were identified based on high levels of expression at the BBB and ability to transport antibodies specific for the target across the BBB. Further, monospecific and multispecific antibodies against these BBB-R targets were generated. Using those antibodies, basigin, Glut1 and CD98hc were shown to be candidate targets on the BBB for transporting agents (e.g., therapeutic and/or imaging agents) across the BBB.

In one aspect of the present disclosure, provided herein is a method of transporting an agent across the blood-brain barrier. The method can include exposing the blood-brain barrier to an antibody which (i) binds to a blood-brain barrier receptor (BBB-R); and (ii) is coupled to the agent; wherein the antibody, upon binding to the BBB-R, transports the agent coupled thereto across the blood-brain barrier. In some aspects, the BBB-R is CD98 heavy chain (CD98hc). In some aspects, the BBB-R is basigin. In some aspects of this method, the BBB-R is Glucose Transporter Type 1 (Glut1). In some aspects of this method, the blood-brain barrier is in a mammal. In some aspects of this method, the mammal has a neurological disease or disorder.

In another aspect of the present disclosure, provided herein is a method of treating a neurological disease or disorder in a mammal. The method can include administering to the mammal an antibody which (i) binds to a BBB-R; and (ii) is coupled to a therapeutic agent which is effective for treating the neurological disease or disorder. In some aspects of the method of treatment, the BBB-R is CD98hc. In some aspects of the method of treatment, the BBB-R is basigin. In some aspects of the method of treatment, the BBB-R is Glutl. In some aspects of the method of treatment, the neurological disease or disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

In certain aspects of the above methods, the mammal can be a human. In certain aspects of the above methods, the agent can be an imaging agent. In any of the above aspects, the agent can be a neurological disorder drug. In certain aspects of the above methods, binding of the antibody to the BBB-R does not impair binding of the BBB-R to one or more of its native ligands. In certain aspects of the above methods, binding of the BBB-R to one or more of its native ligands in the presence of the antibody is at least 80% of the amount of binding in the absence of the antibody. In certain aspects of the above methods, binding of the antibody to the BBB-R does not impair transport of any of the native ligands of the BBB-R across the blood-brain barrier. In certain aspects of the above methods, transport of any of the native ligands of the BBB-R across the blood-brain barrier is at least 80% of the amount of transport in the absence of the antibody.

In certain aspects of the above methods, the antibody has been engineered to have a low binding affinity.

In certain aspects of the above methods, the antibody does not inhibit cell proliferation, cell division, and/or cell adhesion. In certain aspects of the above methods, the antibody does not induce cell death. In certain aspects of the above methods, the antibody has an IC₅₀ for the BBB-R from about 1 nM to about 100 jaM, from about 1 nM to about 10 nM, from about 5 nM to about 100 jaM, from about 50 nM to about 100 jaM, or from about 100 nM to about 100 aM.

In certain aspects of the above methods, the antibody has an affinity for the BBB-R from about 1 nM to about 10 CjM, from about 1 nM to about 1 jaM, from about 1 nM to about 500 nM, from about 1 nM to about 50 nM, from about 1 nM to about 100 aM.

In certain aspects of the above methods, the antibody is administered to the mammal at a therapeutic dose. In some aspects, the therapeutic dose is BBB-R-saturating.

In certain aspects of the above methods, the antibody is multispecific, and the agent coupled thereto comprises an antigen-binding site of the multispecific antibody which binds to a brain antigen. In some aspects, the multispecific antibody is bispecific. In some aspects, the brain antigen is selected from the group consisting of: beta-secretase 1 (BACE1), Abeta, epidermal growth factor receptor (EGFR), human epidermal growth factor receptor 2 (HER2), Tau, apolipoprotein (e.g., apolipoprotein E4 (ApoE4)), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6. In some aspects, the multispecific antibody binds both CD98hc and BACEI. In some aspects, the multispecific antibody binds both CD98hc and Abeta. In some aspects, the multispecific antibody binds both basigin and BACEI. In some aspects, the multispecific antibody binds both basigin and Abeta. In some aspects, the multispecific antibody binds both Glut1 and BACEI. In some aspects, the multispecific antibody binds both Glut1 and Abeta. In some aspects, the BBB-R is CD98hc. In some aspects, the BBB-R is basigin.

In one aspect, the present disclosure provides anti-basigin (anti-Bsg) antibodies which are suitable for use in the methods of transporting agents across the blood-brain barrier as provided herein. In some embodiments, an anti-Bsg antibody is provided wherein binding of the antibody to Bsg does not impair binding of basigin to one or more of its native ligands. In certain embodiments, an anti-Bsg antibody is provided wherein the amount of binding of Bsg to one or more of its native ligands in the presence of the antibody is at least 80% of the amount of binding of Bsg to the one or more native ligands in the absence of the antibody.

In some embodiments, an anti-Bsg antibody is provided wherein binding of the antibody to Bsg does not impair transport of one or more of Bsg's native ligands across the blood brain barrier. In certain embodiments, an anti-Bsg antibody is provided wherein the amount of transport across the blood brain barrier of one or more of Bsg's native ligands in the presence of the antibody is at least 80% of the amount of transport across the blood-brain barrier of one or more of the native ligands in the absence of the antibody.

In some embodiments, an anti-Bsg antibody of the present disclosure is specific for basigin from one or more species. In some embodiments, anti-Bsg antibodies provided herein specifically bind murine Bsg (mBsg). In some embodiments, anti-Bsg antibodies provided herein specifically bind human Bsg (hBsg). In some embodiments, anti-Bsg antibodies provided herein are capable of specifically binding hBsg and mBsg.

As described further below, there are multiple isoforms of Bsg known. Accordingly, in some embodiments, an anti-Bsg antibody of the present disclosure is isoform specific. In some embodiments, an anti-Bsg antibody of the present disclosure specifically binds an isoform of hBsg, e.g., hBsg isoform 1 (hBsg1), hBsg isoform 2 (hBsg2). For example, an anti-Bsg antibody of the present disclosure is an anti-hBsg2 antibody. In some embodiments, an anti-Bsg antibody of the present disclosure specifically binds an isoform of mBsg. In certain embodiments, an anti-Bsg antibody of the present disclosure binds to an epitope within the extracellular domain of Bsg.

In some aspects, the present disclosure provides anti-Bsg antibodies comprising complementarity determing regions (CDRs), framework regions (FRs), and/or light and heavy chain variable domains having amino acids as described herein. In some embodiments,

In certain embodiments, an anti-Bsg antibody of the present disclosure comprises a light chain CDR1 amino acid sequence selected from SEQ ID NOs:3, 19, 35, 51, and 67, a light chain CDR2 amino acid sequence selected from SEQ ID NOs:4, 20, 36, 52, and 68, and a light chain CDR3 amino acid sequence selected from SEQ ID NOs:5, 21, 37, 53, and 69.

In certain embodiments, an anti-Bsg antibody comprises a heavy chain CDR1 amino acid sequence selected from SEQ ID NOs:6, 22, 38, 54, and 70, a heavy chain CDR2 amino acid sequence selected from SEQ ID NOs:7, 23, 39, 55, and 71, and a heavy chain CDR3 amino acid sequence selected from SEQ ID NOs:8, 24, 40, 56, and 72.

In certain embodiments, an anti-Bsg antibody further comprises light chain variable domain framework regions comprising an amino acid sequence selected from SEQ ID NOs: 9, 25, 41, 57, and 73 for FR1, an amino acid sequence selected from SEQ ID NOs: 10. 26, 42, 58, and 74 for FR2, an amino acid sequence selected from SEQ ID NOs: 11, 27, 43, 59, and 75 for FR3, and an amino acid sequence selected from SEQ ID NOs: 12, 28, 44, 60, and 76 for FR4.

In certain embodiments, an anti-Bsg antibody further comprises heavy chain variable domain framework regions comprising an amino acid sequence selected from SEQ ID NOs: 13, 29, 45, 61, and 77 for FR1, an amino acid sequence selected from SEQ ID NOs:14. 30, 46, 62, and 78 for FR2, an amino acid sequence selected from SEQ ID NOs:15, 31, 47, 63, and 79 for FR3, and an amino acid sequence selected from SEQ ID NOs: 16, 32, 48, 64, and 80 for FR4.

In certain embodiments, an anti-Bsg antibody comprises a light chain comprising a variable domain comprising an amino acid sequence selected from SEQ ID NOs:1, 17, 33, 49, and 65.

In certain embodiments, an anti-Bsg antibody comprises a light chain variable domain comprising an amino acid sequence that is at least 90%, at least 91%, at least 92%, at least 93%, at least 94%, at least 95%, at least 96%, at least 97%, at least 98%, or at least 99% identical to an amino acid sequence selected from SEQ ID NOs:1, 17, 33, 49, and 65.

In certain embodiments, an anti-Bsg antibody comprises a heavy chain comprising a variable domain comprising an amino acid sequence selected from SEQ ID NOs:2, 18, 34, 50, and 66.

In certain embodiments, an anti-Bsg antibody comprises a heavy chain variable domain comprising an amino acid sequence that is at least 90%, at least 91%, at least 92%, at least 93%, at least 94%, at least 95%, at least 96%, at least 97%, at least 98%, or at least 99% identical to an amino acid sequence selected from SEQ ID NOs:2, 18, 34, 50, and 66.

In certain embodiments, an anti-Bsg antibody comprises a light chain variable domain comprising an amino acid sequence selected from SEQ ID NOs:1, 17, 33, 49, and 65 and a heavy chain variable domain comprising an amino acid sequence selected from SEQ ID NOs:2, 18, 34, 50, and 66.

In some embodiments, an anti-Bsg antibody comprises a light chain comprising an amino acid sequence corresponding to SEQ ID NO: 1 and a heavy chain comprising an amino acid sequence corresponding to SEQ ID NO:2. In one embodiment, the anti-Bsg antibody is anti-BsgA.

In some embodiments, an anti-Bsg antibody comprises a light chain comprising an amino acid sequence corresponding to SEQ ID NO: 17 and a heavy chain comprising an amino acid sequence corresponding to SEQ ID NO: 18. In one embodiment, the anti-Bsg antibody is anti-BsgB.

In some embodiments, an anti-Bsg antibody comprises a light chain comprising an amino acid sequence corresponding to SEQ ID NO:33 and a heavy chain comprising an amino acid sequence corresponding to SEQ ID NO:34. In one embodiment, the anti-Bsg antibody is anti-BsgC.

In some embodiments, an anti-Bsg antibody comprises a light chain comprising an amino acid sequence corresponding to SEQ ID NO:49 and a heavy chain comprising an amino acid sequence corresponding to SEQ ID NO:50. In one embodiment, the anti-Bsg antibody is anti-BsgD.

In some embodiments, an anti-Bsg antibody comprises a light chain comprising an amino acid sequence corresponding to SEQ ID NO:65 and a heavy chain comprising an amino acid sequence corresponding to SEQ ID NO:66. In one embodiment, the anti-Bsg antibody is anti-BsgE.

In one aspect, the present disclosure provides anti-Glut1 antibodies which are suitable for use in the methods of transporting agents across the blood-brain barrier as provided herein. In some embodiments, an anti-Glut1 antibody is provided wherein binding of the antibody to Glut1 does not impair binding of Glut1 to one or more of its native ligands. In certain embodiments, an anti-Glut1 antibody is provided wherein the amount of binding of Glut1 to one or more of its native ligands in the presence of the antibody is at least 80% of the amount of binding of Glut1 to the one or more native ligands in the absence of the antibody.

In some embodiments, an anti-Glut1 antibody is provided wherein binding of the antibody to Glut1 does not impair transport of one or more of Glut1's native ligands across the blood brain barrier. In certain embodiments, an anti-Glut1 antibody is provided wherein the amount of transport across the blood brain barrier of one or more of Glut1's native ligands in the presence of the antibody is at least 80% of the amount of transport across the blood brain barrier of the one or more native ligands in the absence of the antibody.

In some embodiments, an anti-Glut1 antibody of the present disclosure is specific for basigin from one or more species. In some embodiments, anti-Glut1 antibodies provided herein specifically bind murine Glut1 (mGlut1). In some embodiments, anti-Glut1 antibodies provided herein specifically bind human Glut1 (hGlut1). In some embodiments, anti-Glut1 antibodies provided herein are capable of specifically binding hGlut1 and mGlut1.

In certain embodiments, an anti-Glut1 antibody comprises a light chain CDR1 amino acid sequence comprising SEQ ID NO:83, a light chain CDR2 amino acid sequence comprising SEQ ID NO:84, and a light chain CDR3 amino acid sequence comprising SEQ ID NO:85 and/or a heavy chain CDR1 amino acid sequence comprising SEQ ID NO:86, a heavy chain CDR2 amino acid sequence comprising SEQ ID NO:87, and a heavy chain CDR3 amino acid sequence comprising SEQ ID NO:88.

In certain embodiments, an anti-Glut1 antibody comprises a light chain variable domain comprising framework regions comprising amino acid sequences corresponding to SEQ ID NO: 89 for FR1, SEQ ID NO:90 for FR2, SEQ ID NO:91 for FR3, and SEQ ID NO:92 for FR4.

In certain embodiments, an anti-Glut1 antibody comprises a heavy chain variable domain comprising framework regions comprising amino acid sequences corresponding to SEQ ID NO: 93 for FR1, SEQ ID NO:94 for FR2, SEQ ID NO:95 for FR3, and SEQ ID NO:96 for FR4.

In certain embodiments, an anti-Glut1 antibody comprises a light chain variable domain comprising an amino acid sequence corresponding to SEQ ID NO:81 and a heavy chain variable domain comprising an amino acid sequence corresponding to SEQ ID NO:82.

In certain aspects, the present disclosure provides multispecific antibodies capable of binding a BBB-R. In some embodiments, the multispecific antibody is a bispecific antibody. In some embodiments, the multispecific antibody comprises a first antigen binding site from any of the anti-BBB-R antibodies disclosed herein. In some embodiments, the multispecific antibody disclosed herein further comprise a second antigen binding site capable of binding a brain antigen as disclosed herein. In certain embodiments, the brain antigen is selected from the group consisting of: BASCE1, Abeta, EGFR, HER2, Tau, apolipoprotein (e.g., ApoE4), alpha-synuclein, CD20, huntingtin, PrP, LRRK2, parkin, presenilin 1, presenilin 2, gamma secretase, DR6, APP, p75NTR, and caspase.

In another aspect, the present disclosure provides nucleic acids encoding any of the polypeptides disclosed herein, including any of the antibodies provided.

In another aspect, the present disclosure provides host cells comprising such nucleic acids and methods of producing the antibodies disclosed herein. Accordingly, provided herein are methods for producting an antibody comprising culturing a host cell so as to produce an antibody of the present disclosure.

In another aspect, the present disclosure provides pharmaceutical compositions comprising one or more of the antibodies disclosed herein.

### BRIEF DESCRIPTION OF THE FIGURES

FIG. 1A depicts the screening cascade used to determine success of potential receptor-mediated transport targets.

FIG. 1B depicts binding of naïve phage library-derived anti-Lrp1 and anti-insulin receptor (InsR) antibodies to their corresponding murine receptors transfected in HEK293 cells by flow cytometry (the peak to the left in each histogram is the control antibody (“2^{nd} Ab-PE”) and the peak to the right is the anti-Lrp1 (left histogram) or anti-InsR (right histogram) antibody).

FIG. 1C is a line graph quantifying brain uptake of trace doses of I¹²⁵-labeled antibodies (anti-Transferrin receptor (TfR^{A}), anti-Lrp1, and anti-InsR) at various time points post-dose after intravenous administration in wild-type mice, quantified as mean±SEM percent injected dose per gram of brain tissue (n=3 per group and time point).

FIG. 1D is a bar graph quantifying antibody concentration in brain 1 and 24 hours after a 20 mg/kg dose of the indicated antibody. Bar graphs represent mean±SEM (n=6 per group and time point; *P<0.05, **P<0.01, ***P<0.001, ****P<0.0001).

FIG. 1E contains photographs of mouse cortical tissue sections following immunohistochemical staining, and depicts antibody localization 1 hour after a 5 mg/kg intravenous injection of the indicated antibody. Scale bar, 50 am.

FIG. 2A depicts genes that were enriched at the BBB as determined using microarray expression profiling of FACS-purified BBB and liver/lung endothelial cells from wild-type mice (described in Tam et al., Dev Cell. 2012 Feb. 14; 22(2):403-17).

FIG. 2B depicts flow cytometry analysis of anti-Lrp8, anti-Ldlrad3 and anti-CD320 antibodies and shows binding of the antibodies to their corresponding antigens expressed in HEK293 cells. The peak to the left in each histogram corresponds to the control antibody and the peak to the right corresponds to anti-Lrp8, anti-Ldlrad3 and anti-CD320 antibodies (from left to right histogram).

FIG. 2C is a line graph quantifying brain uptake of trace doses of I¹²⁵-labeled antibodies at various time points post-dose after intravenous administration in wild-type mice of the indicated antibodies. The data are quantified as mean±SEM percent injected dose per gram of brain tissue (n=3 per group and time point).

FIGS. 2D and 2E show bar graphs quantifying antibody concentration in brain 1 and 24 hours after a 20 mg/kg dose of the indicated antibody. Bar graphs represent mean±SEM (n=6 per group and time point; ****P<0.001, *P<0.05); “n.s.”, not statistically significant.

FIG. 2F contains photographs of mouse cortical tissue sections following immunohistochemical staining, and depicts antibody localization 1 hour after a 5 mg/kg intravenous injection of the indicated antibody. Scale bar, 50 μm.

FIG. 2G is a bar graph quantifying the average RPKM values (gene expression) generated from RNA-seq data of purified endothelial cells for commonly studied receptors for RMT (i.e., Tfrc, Lrp1, Insr) and BBB-enriched genes identified by microarray (i.e., Lrp8, Ldlrad3, CD320). The dataset revealed low absolute mRNA expression of Lrp8, Ldlrad3, and CD320 on brain endothelial cells.

FIG. 3A depicts the method used to isolate CD31-positive and CD45-negative brain endothelial cells (BECs) from wild-type mice by FACS as previously described (Tam et al., 2012, supra). The isolated BECs were analyzed by mass spectrometry (MS) and the results are shown in FIGS. 3B and 3C.

FIG. 3B is a bar graph quantifying the integrated intensity for the top three most abundant peptide hits as determined by MS for each endothelial cell protein (PgP, Glut1, ZO-1, Esam, Claudin5), compared to other brain cell-specific proteins (Fasn, Aldoc, Glul, Plp1) in brain endothelial cells (BEC) compared to non-BEC.

FIG. 3C is a bar graph quantifying the integrated intensity for the top three most abundant peptide hits for the indicated RMT targets.

FIG. 3D is a table summarizing potential RMT targets identified by literature, microarray, RNA-seq, and mass spectrometry.

FIG. 4A contains histograms quantifying binding, as determined by flow cytometry analysis, of anti-Bsg^{A} and anti-Bsg^{B} binding to HEK293 cells transfected with murine basigin. In each histogram, the left peak corresponds to the control antibody and the peak to the right corresponds to the anti-Basigin antibody.

FIG. 4B contains photographs of mouse cortical tissue following immunohistochemical staining and depicts antibody localization 1 hour after a 5 mg/kg intravenous injection of anti-Bsg^{A} or anti-Bsg^{B}. Scale bar, 50 am.

FIG. 4C is a line graph quantifying brain uptake in wild-type mice of trace doses of the indicated I¹²⁵-labeled anti-basigin antibodies at the indicated time points (in hours (hr)) post-dose after intravenous administration, quantified as mean±SEM percent injected dose per gram of brain tissue (n=3 per group and time point).

FIG. 4D and FIG. 4E are bar graphs quantifying the levels of the indicated antibodies in brain and plasma, respectively, 1 and 24 hours after a 20 mg/kg dose of the indicated antibody. Bar graphs represent mean±SEM (n=6 per group and time point; *P≦0.05, **P≦0.01, ****P≦0.0001).

FIG. 4F depicts results of a competitive ELISA comparison of bivalent (monospecific) anti-Bsg (solid) vs. bispecific (monovalent anti-Bsg) anti-Bsg/BACE1 antibodies (dashed) binding to murine basigin (IC₅₀: anti-Bsg^{A}-7.1 nM, anti-Bsg^{A}/BACE1-105.5 nM, anti-Bsg^{B}-17.5 nM, anti-Bsg^{B}/BACE1-126.6 nM).

FIGS. 4G-4I are bar graphs quantifying brain antibody concentration (nM), brain Aβ concentration (pg/g) and plasma antibody concentration (μM), respectively, 24 hours after a 50 mg/kg intravenous administration of anti-Bsg/BACE1 antibodies or control IgG. Bar graphs represent mean±SEM (n=6 per group and time point; **P≦0.01, ****P≦0.0001), “n.s.”, not statistically significant.

FIG. 5A is flow cytometry histogram depicting binding of the anti-Glut1 binding to HEK293 cells stably expressing Glut1. The leftmost peak corresponds to control antibody.

FIG. 5B is a photograph of mouse cortical tissue sections following immunohistochemical staining, and depicts antibody localization 1 hour after a 5 mg/kg intravenous injection of anti-Glut1 antibody. Scale bar, 50 μm.

FIG. 5C is a line graph quantifying brain uptake of trace doses of I¹²⁵-labeled anti-Glut1 at various time points post-dose after intravenous administration in wild-type mice, quantified as mean±SEM percent injected dose per gram of brain tissue (n=3 per group and time point).

FIG. 5D and FIG. 5E are line graphs quantifying the antibody levels in brain and plasma, respectively, days after a 20 mg/kg dose of the indicated antibody.

FIG. 5F is a line graph quantifying the mean fluorescence intensity (MFI) determined by flow cytometry analysis of the bivalent (monospecific) anti-Glut1 (solid) vs. the bispecific (monovalent anti-Glut1) anti-Glut1/BACE1 (dashed) binding to the HEK293 cells stably expressing Glut1. (EC50: anti-Glut1-0.6 μg/mL, anti-Glut1/BACE1->10 g/mL).

FIGS. 5G, 5H, 5I, and 5J are line graphs quantifying plasma antibody concentration, brain antibody concentration, brain Aβ levels, and plasma Aβ levels, respectively, days after a single 50 mg/kg intravenous administration of anti-Glut1/BACE1 or control IgG.

FIGS. 5K and 5L contain bar graphs quantifying the amount of antibody, expressed as percent (%) injected dose per gram of brain (FIG. 5K) or brain antibody concentration (FIG. 5L), in brain 1 and 24 hours after a 20 mg/kg dose of the indicated antibody. *P≦0.05, **P≦0.01, ***P≦0.001,****P≦0.0001 compared to control IgG at the same time point).

FIG. 6A is a flow cytometry analysis of the anti-CD98hc antibody binding to the HEK293 cells stably expressing CD98hc. In each histogram, the control antibody (2^{nd} Ab-PE) corresponds to the leftmost peak and the anti-CD98hc antibody corresponds to the rightmost peak.

FIG. 6B contains photographs of mouse cortical tissue sections following immunohistochemical staining, and depicts antibody localization 1 hour after a 5 mg/kg intravenous injection of anti-CD98hc^{A} or anti-CD98hcB. Scale bar, 50 μm.

FIG. 6C is a line graph quantifying brain uptake (% injected dose/gram brain) of trace doses of I¹²⁵-labeled anti-CD98hc antibodies (or IgG control or anti-TfR antibody) at various time points post-dose after intravenous administration in wild-type mice, quantified as mean±SEM percent injected dose per gram of brain tissue (n=3 per group and time point).

FIGS. 6D and 6E are bar graph quantifying antibody levels in brain (% injected dose/gram brain) and brain-to-plasma ratio, respectively, 1 and 24 hours after a 20 mg/kg dose of the indicated antibody. Bar graphs represent mean±SEM (n=6 per group and time point; ****P≦0.0001), “n.s.”, not statistically significant.

FIG. 6F is a line graph quantifying the affinities (expressed as normalized OD650) of parental bivalent (monospecific) anti-CD98hc antibodies compared to anti-CD98hc/BACE1 bispecific antibodies, as measured by flow cytometry with HEK293 cells expressing murine CD98hc (IC₅₀: anti-CD98hc^{A}-1.5 nM, anti-CD98hcA/BACE1-4.0 nM anti-CD98hc^{B}-4.6 nM, anti-CD98hc^{B}/BACE1-164.4 nM).

FIGS. 6G, 6H, 6I, and 6J, are graphs quantifying plasma antibody concentration, brain antibody concentration, brain Aβ levels, and plasma Aβ levels, respectively, at the indicated number of days post-dose after a 50 mg/kg intravenous administration of the indicated anti-CD98hc/BACE1 antibodies or control IgG. Bar graphs represent mean±SEM (n=5 per group and time point, **P≦0.01, ***P≦0.001, ****P≦0.0001; “n.s”, not statistically significant). In FIG. 6I, the columns in each time point (1 and 4 days post dose), ordered from left to right, correspond to: control IgG, anti-CD98hc^{A}/BACE1, and anti-CD98hc^{B}/BACE1.

FIG. 6K is a line graph showing brain uptake of trace doses of the indicated I¹²⁵-labeled antibodies at various time points (hours (hr)) post-dose after intravenous administration in wild-type mice, quantified as mean±SEM percent injected dose per gram of brain tissue (n=3 per group and time point).

FIG. 6L is a bar graph quantifying brain antibody concentration at the indicated time point (1 or 24 hours) post-dose after a 50 mg/kg intravenous administration of anti-CD98hc/BACE1 antibodies, anti-TfR^{A} antibody, or control IgG. Bar graphs represent mean±SEM (n=5 per group and time point, **P≦0.01, ***P≦0.001, ****P≦0.0001; “n.s”, not statistically significant).

FIGS. 6M and 6N are graphs quantifying percent (%) Aβₓ₋₄₀ reduction compared to control IgG (FIG. 6M) and Aβₓ₋₄₀ concentrations in brain (FIG. 6N) at the indicated number of days post-dose after a single 50 mg/kg intravenous administration of the indicated anti-CD98hc/BACE1 antibody or control IgG.

FIGS. 6O and 6P are graphs quantifying plasma antibody concentration (FIG. 6O) and brain antibody concentration (FIG. 6P) at the indicated number of days post-dose after a 50 mg/kg intravenous administration of the indicated anti-CD98hc/BACE1 antibody or control IgG. Error bars represent mean±SEM (n=5 per group and time point).

FIG. 7A is a photograph of a Western blot. Wild type IMCD3 cells were treated with the indicated antibodies and concentrations (μM) for 24 hours. Lysates were probed for endogenous CD98hc and actin as the loading control.

FIG. 7B is a bar graph quantifying the Western blot data represented in FIG. 7A. The Western blot data are averaged from 3 independent experiments each performed in triplicate. Bars represent mean±SEM (n=3).

FIG. 7C contains photographs of IMCD3 cells stably overexpressing mouse CD98hc treated with 1 μM of the indicated antibodies for 1 hour at 37° C. The cells were fixed, and stained for human IgG, mouse CD98hc, and lysosomal marker, Lamp1. The images are representative of cellular uptake of control IgG, anti-CD98hc^{A}/BACE1, and anti-CD98hc^{B}/BACE1 co-stained with Lamp1. Scale bar=5 am.

FIG. 7D is a bar graph quantifying CD98hc puncta. The puncta were analyzed and quantified for co-localization with Lamp1. Bars represent mean±SEM (n=5).

FIGS. 7E-H are photographs of Western blot results. The blots were analyzed for CD98hc expression in brain lysates after a single 50 mg/kg dose of the indicated antibodies at various days post-dose (n=5 per group and time point).

FIG. 7I is a bar graph quantifying CD98hc levels in the Western blots shown in FIGS. 7E-7H. All graphs represent mean±SEM (n=5 per group and time point).

FIG. 7J is a bar graph quantifying percent (%) amino acid uptake activity. IMCD3 cells stably overexpressing mouse CD98hc cells were treated with 1 aM of the indicated antibodies for 24 hours and amino acid uptake activity was assessed by the amount total internalized HPG, a methionine analog. BCH (2-amino-2-norbornane-carboxylic acid), an inhibitor of a system L amino acid transporter, was used as a positive control. Methionine uptake was expressed as a percentage of control IgG and plotted against each data point. Bars represent mean±SEM (n=12).

FIG. 8 is a bar graph quantifying the parenchyma antibody concentration (ng/mL) per mg of total protein in brains of mice injected with the indicated antibody. Antibody concentrations from parenchyma lysates were assessed by a human IgG ELISA and normalized to total protein concentrations. n=5 per group, bar graphs shown are mean±SEM. **p≦0.01 and ****p≦0.0001 by ANOVA versus Control IgG (by Dunnett's post-hoc test).

FIG. 9 is a table summarizing the affinities of the indicated RMT antibodies. All affinities were determined by Biacore except the anti-Glut1 antibody, which was evaluated using FACS analysis.

FIG. 10 contains microscopic images taken to evaluate CD98hc subcellular localization and trafficking. Mouse primary brain endothelial cells were fixed and stained with subcellular vesicular markers (left panels) and anti-mouse CD98hc (center panels). Endogenous CD98hc was localized to the plasma membrane (arrows) and also found in intracellular puncta (arrowheads). Colocalization was examined by co-staining with anti-caveolin1 (A), anti-TfR (B), or anti-EEA1 (C). On the plasma membrane, a subset of CD98hc is colocalized with caveolin1 (arrows in merged panel A). Some intracellular puncta are colocalized with caveolin1 (arrowheads in panel A). Very few puncta are colocalized with TfR as shown in (B) and the merged image. Some CD98hc intracellular puncta are colocalized with EEA1 (arrowheads in merged panel C). Scale bar=5 μM.

### DETAILED DESCRIPTION OF EMBODIMENTS OF THE INVENTION

### I. Definitions

The “blood-brain barrier” or “BBB” refers to the physiological barrier between the peripheral circulation and the brain and spinal cord (i.e., the CNS) which is formed by tight junctions within the brain capillary endothelial plasma membranes, creating a tight barrier that restricts the transport of molecules into the brain, even very small molecules such as urea (60 Daltons). The blood-brain barrier within the brain, the blood-spinal cord barrier within the spinal cord, and the blood-retinal barrier within the retina are contiguous capillary barriers within the CNS, and are herein collectively referred to as the blood-brain barrier or BBB. The BBB also encompasses the blood-CSF barrier (choroid plexus) where the barrier is comprised of ependymal cells rather than capillary endothelial cells.

A “blood-brain barrier receptor” (abbreviated “BBB-R” herein) is a transmembrane receptor protein expressed on brain endothelial cells which is capable of transporting molecules across the blood-brain barrier. As discussed above, one strategy to increase brain penetration of large molecule drugs is to utilize transcytosis trafficking pathways of BBB-R, e.g. using antibodies that target those receptors. The present disclosure provides novel BBB-R targets and methods of transporting agents across the BBB into the brain using monoclonal antibodies against those BBB-R. Such BBB-R include CD98 heavy chain (CD989hc), glucose transporter 1 (Glutl), and basigin (Bsg).

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In certain embodiments, an individual who may be administered and/or treated with an antibody disclosed herein is an individual who has not been diagnosed with cancer. In certain embodiments, an individual who may be administered and/or treated with an antibody disclosed herein is an individual who has not been diagnosed with brain cancer. In certain embodiments, an individual who may be administered and/or treated with an antibody disclosed herein is an individual who does not have cancer. In certain embodiments, an individual who may be treated and/or administered with an antibody disclosed herein is an individual who does not have brain cancer. The “central nervous system” or “CNS” refers to the complex of nerve tissues that control bodily function, and includes the brain and spinal cord.

The terms “amyloid beta,” “beta-amyloid,” “Abeta,” “amyloidpβ,” and “Aβ”, used interchangeably herein, refer to the fragment of amyloid precursor protein (“APP”) that is produced upon β-secretase 1 (“BACE1”) cleavage of APP, as well as modifications, fragments and any functional equivalents thereof, including, but not limited to, Aβ1-40, and Aβ1-42. Aβ is known to exist in monomeric form, as well as to associate to form oligomers and fibril structures, which may be found as constituent members of amyloid plaque. The structure and sequences of such Aβ peptides are well known to one of ordinary skill in the art and methods of producing said peptides or of extracting them from brain and other tissues are described, for example, in Glenner and Wong, Biochem Biophys Res. Comm. 129: 885-890 (1984). Moreover, Aβ peptides are also commercially available in various forms.

The term “cerebral vasogenic edema” refers to an excess accumulation of intravascular fluid or protein in the intracellular or extracellular spaces of the brain. Cerebral vasogenic edema is detectable by, e.g., brain MRI, including, but not limited to FLAIR MRI, and can be asymptomatic (“asymptomatic vasogenic edema”) or associated with neurological symptoms, such as confusion, dizziness, vomiting, and lethargy (“symptomatic vasogenic edema”) (see Sperling et al. Alzheimer's & Dementia, 7:367, 2011).

The term “cerebral microhemorrhage” refers to an intracranial hemorrhage, or bleeding in the brain, of an area that is less than about 1 cm in diameter. Cerebral microhemorrhage is detectable by, e.g., brain MRI, including, but not limited to T2*-weighted GRE MRI, and can be asymptomatic (“asymptomatic microhemorrhage”) or can potentially be associated with symptoms such as transient or permanent focal motor or sensory impairment, ataxia, aphasia, and dysarthria (“symptomatic microhemorrhage”). See, e.g., Greenberg, et al., 2009, Lancet Neurol. 8:165-74.

The term “sulcal effusion” refers to an effusion of fluid in the furrows, or sulci, of the brain. Sulcal effusions are detectable by, e.g., brain MRI, including but not limited to FLAIR MRI. See Sperling et al. Alzheimer's & Dementia, 7:367, 2011.

The term “superficial siderosis of the central nervous system” refers to bleeding or hemorrhage into the subarachnoid space of the brain and is detectable by, e.g., brain MRI, including but not limited to T2*-weighted GRE MRI. Symptoms indicative of superficial siderosis of the central nervous system include sensorineural deafness, cerebellar ataxia, and pyramidal signs. See Kumara-N, Am J Neuroradiol. 31:5, 2010.

The term “amyloidosis,” as used herein, refers to a group of diseases and disorders caused by or associated with amyloid or amyloid-like proteins and includes, but is not limited to, diseases and disorders caused by the presence or activity of amyloid-like proteins in monomeric, fibril, or polymeric state, or any combination of the three, including by amyloid plaques. Such diseases include, but are not limited to, secondary amyloidosis and age-related amyloidosis, such as diseases including, but not limited to, neurological disorders such as Alzheimer's Disease (“AD”), diseases or conditions characterized by a loss of cognitive memory capacity such as, for example, mild cognitive impairment (MCI), Lewy body dementia, Down's syndrome, hereditary cerebral hemorrhage with amyloidosis (Dutch type), the Guam Parkinson-Demential complex and other diseases which are based on or associated with amyloid-like proteins such as progressive supranuclear palsy, multiple sclerosis, Creutzfeld Jacob disease, Parkinson's disease, HIV-related dementia, ALS (amyotropic lateral sclerosis), inclusion-body myositis (IBM), adult onset diabetes, endocrine tumor and senile cardiac amyloidosis, and various eye diseases including macular degeneration, drusen-related optic neuropathy, glaucoma, and cataract due to beta-amyloid deposition.

Glaucoma is a group of diseases of the optic nerve involving loss of retinal ganglion cells (RGCs) in a characteristic pattern of optic neuropathy. RGCs are the nerve cells that transmit visual signals from the eye to the brain. Caspase-3 and Caspase-8, two major enzymes in the apoptotic process, are activated in the process leading to apoptosis of RGCs. Caspase-3 cleaves amyloid precursor protein (APP) to produce neurotoxic fragments, including Abeta. Without the protective effect of APP, Abeta accumulation in the retinal ganglion cell layer results in the death of RGCs and irreversible loss of vision.

Glaucoma is often, but not always, accompanied by an increased eye pressure, which may be a result of blockage of the circulation of aqueous, or its drainage. Although raised intraocular pressure is a significant risk factor for developing glaucoma, no threshold of intraocular pressure can be defined which would be determinative for causing glaucoma. The damage may also be caused by poor blood supply to the vital optic nerve fibers, a weakness in the structure of the nerve, and/or a problem in the health of the nerve fibers themselves. Untreated glaucoma leads to permanent damage of the optic nerve and resultant visual field loss, which can progress to blindness.

The term “mild Alzheimer's Disease” or “mild AD” as used herein (e.g., a “patient diagnosed with mild AD”) refers to a stage of AD characterized by an MMSE score of 20 to 26.

The term “mild to moderate Alzheimer's Disease” or “mild to moderate AD” as used herein encompasses both mild and moderate AD, and is characterized by an MMSE score of 18 to 26.

The term “moderate Alzheimer's Disease” or “moderate AD” as used herein (e.g., a “patient diagnosed with moderate AD”) refers to a stage of AD characterized by an MMSE score of 18 to 19.

A “neurological disorder” as used herein refers to a disease or disorder which affects the CNS and/or which has an etiology in the CNS. Exemplary CNS diseases or disorders include, but are not limited to, neuropathy, amyloidosis, cancer, an ocular disease or disorder, viral or microbial infection, inflammation, ischemia, neurodegenerative disease, seizure, behavioral disorders, and a lysosomal storage disease. For the purposes of this application, the CNS will be understood to include the eye, which is normally sequestered from the rest of the body by the blood-retina barrier. Specific examples of neurological disorders include, but are not limited to, neurodegenerative diseases (including, but not limited to, Lewy body disease, postpoliomyelitis syndrome, Shy-Draeger syndrome, olivopontocerebellar atrophy, Parkinson's disease, multiple system atrophy, striatonigral degeneration, tauopathies (including, but not limited to, Alzheimer disease and supranuclear palsy), prion diseases (including, but not limited to, bovine spongiform encephalopathy, scrapie, Creutzfeldt-Jakob syndrome, kuru, Gerstmann-Straussler-Scheinker disease, chronic wasting disease, and fatal familial insomnia), bulbar palsy, motor neuron disease, and nervous system heterodegenerative disorders (including, but not limited to, Canavan disease, Huntington's disease, neuronal ceroid-lipofuscinosis, Alexander's disease, Tourette's syndrome, Menkes kinky hair syndrome, Cockayne syndrome, Halervorden-Spatz syndrome, lafora disease, Rett syndrome, hepatolenticular degeneration, Lesch-Nyhan syndrome, and Unverricht-Lundborg syndrome), dementia (including, but not limited to, Pick's disease, and spinocerebellar ataxia), cancer (e.g., of the CNS, including brain metastases resulting from cancer elsewhere in the body).

A “neurological disorder drug” is a drug or therapeutic agent that treats one or more neurological disorder(s). Neurological disorder drugs of the invention include, but are not limited to, antibodies, peptides, proteins, natural ligands of one or more CNS target(s), modified versions of natural ligands of one or more CNS target(s), aptamers, inhibitory nucleic acids (e.g., small inhibitory RNAs (siRNA) and short hairpin RNAs (shRNA)), ribozymes, and small molecules, or active fragments of any of the foregoing. Exemplary neurological disorder drugs of the invention are described herein and include, but are not limited to: antibodies, aptamers, proteins, peptides, inhibitory nucleic acids and small molecules and active fragments of any of the foregoing that either are themselves or specifically recognize and/or act upon (e.g., inhibit, activate, or detect) a CNS antigen or target molecule such as, but not limited to, amyloid precursor protein or portions thereof, amyloid beta, beta-secretase, gamma-secretase, tau, alpha-synuclein, parkin, huntingtin, DR6, presenilin, ApoE, glioma or other CNS cancer markers, and neurotrophins. Non-limiting examples of neurological disorder drugs and the disorders they may be used to treat are provided in the following Table A:

TABLE A: Non-Limiting Examples of Neurological Disorder Drugs and the Corresponding Disorders they May be Used to Treat

TABLE A
Non-limiting examples of neurological disorder drugs and the
corresponding disorders they may be used to treat

| Drug | Neurological disorder |
|---|---|
| Anti-BACE1 Antibody | Alzheimer's, acute and |
| injury, stroke | chronic brain |
| Anti-Abeta Antibody | Alzheimer's disease |
| Anti-Tau Antibody | Alzheimer's disease, |
| | tauopathies |
| Neurotrophin | Stroke, acute brain injury, |
| | spinal cord injury |
| Brain-derived neurotrophic | Chronic brain injury |
| factor (BDNF), | (Neurogenesis) |
| Fibroblast growth factor 2 (FGF-2) | |
| Anti-Epidermal Growth Factor | Brain cancer |
| Receptor (EGFR)-antibody | |
| Glial cell-line derived neural factor | Parkinson's disease |
| (GDNF) | |
| Brain-derived neurotrophic factor | Amyotrophic lateral sclerosis, |
| (BDNF) | depression |
| Lysosomal enzyme | Lysosomal storage disorders |
| | of the brain |
| Ciliary neurotrophic factor (CNTF) | Amyotrophic lateral sclerosis |
| Neuregulin-1 | Schizophrenia |
| Anti-HER2 antibody (e.g. | Brain metastasis from HER2- |
| trastuzumab, pertuzumab, etc.) | positive cancer |
| Anti-VEGF antibody (e.g., | Recurrent or newly diagnosed |
| bevacizumab) | glioblastoma, recurrent |
| | malignant glioma, brain |
| | metastasis |

As used herein, an “agent”, e.g., an agent that is delivered across the blood-brain barrier by a BBB-R specific antibody disclosed herein (e.g., anti-CD98hc, anti-Bsg, or anti-Glut1 antibody), is a therapeutic agent or imaging agent. In certain aspects, the therapeutic agent is an antibody (e.g., that is specific for a CNS or brain antigen). In certain aspects, the therapeutic agent is a drug, e.g., a neurological disorder drug, e.g., as described above. In certain aspects, the therapeutic agent is a cytotoxic agent. In certain aspects, the therapeutic agent is an antibody (e.g., the agent (antibody) is one arm of a multispecific antibody).

As used herein, an “imaging agent” is a compound that has one or more properties that permit its presence and/or location to be detected directly or indirectly. Examples of such imaging agents include proteins and small molecule compounds incorporating a labeled moiety that permits detection.

A “CNS antigen” or “brain antigen” is an antigen expressed in the CNS, including the brain, which can be targeted with an antibody or small molecule. Examples of such antigens include, without limitation: beta-secretase 1 (BACE1), amyloid beta (Abeta), epidermal growth factor receptor (EGFR), human epidermal growth factor receptor 2 (HER2), tau, an apolipoprotein, e.g., apolipoprotein E4 (ApoE4), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p⁷⁵ neurotrophin receptor (p75NTR), interleukin 6 receptor (IL6R), TNF receptor 1 (TNFR1), interleukin 1 beta (IL1β), and caspase 6. In a specific embodiment, the antigen is BACE1.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

As used herein, “specifically binding” or “binds specifically to” refers to an antibody selectively or preferentially binding to an antigen. The binding affinity is generally determined using a standard assay, such as Scatchard analysis, or surface plasmon resonance technique (e.g. using BIACORE®).

An “antibody fragment” refers to a molecule other than an intact antibody that comprises a portion of an intact antibody that binds the antigen to which the intact antibody binds. Examples of antibody fragments are well known in the art (see, e.g., Nelson, MAbs (2010) 2(1): 77-83) and include but are not limited to Fab, Fab′, Fab′-SH, F(ab′)₂, and Fv; diabodies; linear antibodies; single-chain antibody molecules including but not limited to single-chain variable fragments (scFv), fusions of light and/or heavy-chain antigen-binding domains with or without a linker (and optionally in tandem); and monospecific or multispecific antigen-binding molecules formed from antibody fragments (including, but not limited to multispecific antibodies constructed from multiple variable domains which lack Fc regions).

The term “monoclonal antibody” as used herein refers to an antibody obtained from a population of substantially homogeneous antibodies, i.e., the individual antibodies comprising the population are identical and/or bind the same epitope, except for possible variants, e.g., containing naturally occurring mutations or that may arise during production of the monoclonal antibody, such variants generally being present in minor amounts. In contrast to polyclonal antibody preparations, which typically include different antibodies directed against different determinants (epitopes), each monoclonal antibody of a monoclonal antibody preparation is directed against a single determinant on the antigen. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including but not limited to the hybridoma method (see, e.g., Kohler et al., Nature, 256:495 (1975)), recombinant DNA methods (see, e.g., U.S. Pat. No. 4,816,567), phage-display methods (e.g., using the techniques described in Clackson et al., Nature, 352:624-628 (1991) and Marks et al., J. Mol. Biol., 222:581-597 (1991)), and methods utilizing transgenic animals containing all or part of the human immunoglobulin loci, such methods and other exemplary methods for making monoclonal antibodies being described herein. Specific examples of monoclonal antibodies herein include chimeric antibodies, humanized antibodies, and human antibodies, including antigen-binding fragments thereof.

The monoclonal antibodies herein specifically include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; Morrison et al., Proc. Natl. Acad. Sci. USA, 81:6851-6855 (1984)).

The term “multispecific antibody” is used in the broadest sense and specifically covers an antibody comprising an antigen-binding domain that has polyepitopic specificity (i.e., is capable of specifically binding to two, or more, different epitopes on one biological molecule or is capable of specifically binding to epitopes on two, or more, different biological molecules).

A “bispecific antibody” is a multispecific antibody comprising an antigen-binding domain that is capable of specifically binding to two different epitopes on one biological molecule or is capable of specifically binding to epitopes on two different biological molecules. A bispecific antibody may also be referred to herein as having “dual specificity” or as being “dual specific.”

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. An exemplary competition assay is provided herein.

The term “chimeric” antibody refers to an antibody in which a portion of the heavy and/or light chain is derived from a particular source or species, while the remainder of the heavy and/or light chain is derived from a different source or species.

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some embodiments, the number of amino acid changes are 10 or less, 9 or less, 8 or less, 7 or less, 6 or less, 5 or less, 4 or less, 3 or less, or 2 or less. In some embodiments, the VL acceptor human framework is identical in sequence to the VL human immunoglobulin framework sequence or human consensus framework sequence.

The term “cytotoxic agent” as used herein refers to a substance that inhibits or prevents a cellular function and/or causes cell death or destruction. Cytotoxic agents include, but are not limited to, radioactive isotopes (e.g., At²¹¹, I³¹¹, I¹²⁵, Y⁹⁰, Re¹⁸⁶, Re¹⁸⁸, Sm¹⁵³, Bi²¹², P³², Pb²¹² and radioactive isotopes of Lu); chemotherapeutic agents or drugs (e.g., methotrexate, adriamicin, vinca alkaloids (vincristine, vinblastine, etoposide), doxorubicin, melphalan, mitomycin C, chlorambucil, daunorubicin or other intercalating agents); growth inhibitory agents; enzymes and fragments thereof such as nucleolytic enzymes; antibiotics; toxins such as small molecule toxins or enzymatically active toxins of bacterial, fungal, plant or animal origin, including fragments and/or variants thereof; and the various antitumor or anticancer agents disclosed below.

“Effector functions” refer to those biological activities attributable to the Fc region of an antibody, which vary with the antibody isotype. Examples of antibody effector functions include: Clq binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

An “effective amount” of an agent, e.g., a pharmaceutical formulation, refers to an amount effective, at dosages and for periods of time necessary, to achieve the desired therapeutic or prophylactic result.

A “native sequence” protein herein refers to a protein comprising the amino acid sequence of a protein found in nature, including naturally occurring variants of the protein.

The term as used herein includes the protein as isolated from a natural source thereof or as recombinantly produced.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991.

The term “FcRn receptor” or “FcRn” as used herein refers to an Fc receptor (“n” indicates neonatal) which is known to be involved in transfer of maternal IgGs to a fetus through the human or primate placenta, or yolk sac (rabbits) and to a neonate from the colostrum through the small intestine. It is also known that FcRn is involved in the maintenance of constant serum IgG levels by binding the IgG molecules and recycling them into the serum. “FcRn binding region” or “FcRn receptor binding region” refers to that portion of an antibody which interacts with the FcRn receptor. Certain modifications in the FcRn binding region of an antibody increase the affinity of the antibody or fragment thereof, for the FcRn, and also increase the in vivo half-life of the molecule. Amino acid substitutions in one or more of the following amino acid positions 251 256, 285, 290, 308, 314, 385, 389, 428, 434 and 436 increases the interaction of the antibody with the FcRn receptor. Substitutions at the following positions also increases the interaction of an antibody with the FcRn receptor 238, 265, 272, 286, 303, 305, 307, 311, 312, 317, 340, 356, 360, 362, 376, 378, 380, 382, 413, 424 or 434, e.g., substitution of (U.S. Pat. No. 7,371,826).

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3(L3)-FR4.

The terms “full length antibody,” “intact antibody,” and “whole antibody” are used herein interchangeably to refer to an antibody having a structure substantially similar to a native antibody structure or having heavy chains that contain an Fc region as defined herein.

The terms “host cell,” “host cell line,” and “host cell culture” are used interchangeably and refer to cells into which exogenous nucleic acid has been introduced, including the progeny of such cells. Host cells include “transformants” and “transformed cells,” which include the primary transformed cells and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. Examples of “host cells” for producing recombinant antibodies include: (1) mammalian cells, for example, Chinese Hamster Ovary (CHO), COS, myeloma cells (including Y0 and NS0 cells), baby hamster kidney (BHK), Hela and Vero cells; (2) insect cells, for example, sf9, sf21 and Tn5; (3) plant cells, for example plants belonging to the genus Nicotiana (e.g. Nicotiana tabacum); (4) yeast cells, for example, those belonging to the genus Saccharomyces (e.g. Saccharomyces cerevisiae) or the genus Aspergillus (e.g. Aspergillus niger); (5) bacterial cells, for example Escherichia coli cells or Bacillus subtilis cells, etc.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda Md. (1991), vols. 1-3. In one embodiment, for the VL, the subgroup is subgroup kappa I as in Kabat et al., supra. In one embodiment, for the VH, the subgroup is subgroup III as in Kabat et al., supra.

A “humanized” antibody refers to a chimeric antibody comprising amino acid residues from non-human HVRs and amino acid residues from human FRs. In certain embodiments, a humanized antibody will comprise substantially all of at least one, and typically two, variable domains, in which all or substantially all of the HVRs (e.g., CDRs) correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

“Humanized” forms of non-human (e.g., murine) antibodies are chimeric antibodies that contain minimal sequence derived from non-human antibodies. For the most part, humanized antibodies are human antibodies (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. For example, in certain embodiments, a humanized antibody will comprise substantially all of at least one, and typically two, variable domains, in which all or substantially all of the HVRs (e.g., CDRs) correspond to those of a non-human antibody, and all or substantially all of the framework regions (FRs) correspond to those of a human antibody. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In certain embodiments, a humanized antibody will comprise substantially all of at least one, and typically two, variable domains, in which all or substantially all of the hypervariable regions correspond to those of a non-human antibody and all or substantially all of the FRs are those of a human antibody, except for FR substitution(s) as noted above. The humanized antibody optionally also will comprise at least a portion of an antibody constant region, typically that of a human antibody. A “humanized form” of an antibody, e.g., a non-human antibody, refers to an antibody that has undergone humanization. For further details, see Jones et al., Nature 321:522-525 (1986); Riechmann et al., Nature 332:323-329 (1988); and Presta, Curr. Op. Struct. Biol. 2:593-596 (1992).

A “human antibody” herein is an antibody comprising an amino acid sequence structure that corresponds with the amino acid sequence structure of an antibody produced by a human or a human cell or derived from a non-human source that utilizes human antibody repertoires or other human antibody-encoding sequences. This definition of a human antibody specifically excludes a humanized antibody comprising non-human antigen-binding residues. Such antibodies can be identified or made by a variety of techniques, including, but not limited to: production by transgenic animals (e.g., mice) that are capable, upon immunization, of producing human antibodies in the absence of endogenous immunoglobulin production (see, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807)); selection from phage display libraries expressing human antibodies or human antibody fragments (see, for example, McCafferty et al., Nature 348:552-553 (1990); Johnson et al., Current Opinion in Structural Biology 3:564-571 (1993); Clackson et al., Nature, 352:624-628 (1991); Marks et al., J. Mol. Biol. 222:581-597 (1991); Griffith et al., EMBO J. 12:725-734 (1993); U.S. Pat. Nos. 5,565,332 and 5,573,905); generation via in vitro activated B cells (see U.S. Pat. Nos. 5,567,610 and 5,229,275); and isolation from human antibody-producing hybridomas.

Antibodies herein include “amino acid sequence variants” with altered antigen-binding or biological activity. Examples of such amino acid alterations include antibodies with enhanced affinity for antigen (e.g. “affinity matured” antibodies), and antibodies with altered Fc region, if present, e.g. with altered (increased or diminished) antibody dependent cellular cytotoxicity (ADCC) and/or complement dependent cytotoxicity (CDC) (see, for example, WO 00/42072, Presta, L. and WO 99/51642, Iduosogie et al.); and/or increased or diminished serum half-life (see, for example, WO 00/42072, Presta, L.).

An “affinity modified variant” has one or more substituted hypervariable region or framework residues of a parent antibody (e.g. of a parent chimeric, humanized, or human antibody) that alter (increase or reduce) affinity. A convenient way for generating such substitutional variants uses phage display. Briefly, several hypervariable region sites (e.g. 6-7 sites) are mutated to generate all possible amino substitutions at each site. The antibody variants thus generated are displayed in a monovalent fashion from filamentous phage particles as fusions to the gene III product of M13 packaged within each particle. The phage-displayed variants are then screened for their biological activity (e.g. binding affinity). In order to identify candidate hypervariable region sites for modification, alanine scanning mutagenesis can be performed to identify hypervariable region residues contributing significantly to antigen binding. Alternatively, or additionally, it may be beneficial to analyze a crystal structure of the antigen-antibody complex to identify contact points between the antibody and its target. Such contact residues and neighboring residues are candidates for substitution according to the techniques elaborated herein. Once such variants are generated, the panel of variants is subjected to screening and antibodies with altered affinity may be selected for further development.

The antibody herein may be conjugated with a “heterologous molecule” for example to increase half-life or stability or otherwise improve the antibody. For example, the antibody may be linked to one of a variety of non-proteinaceous polymers, e.g., polyethylene glycol (PEG), polypropylene glycol, polyoxyalkylenes, or copolymers of polyethylene glycol and polypropylene glycol. Antibody fragments, such as Fab′, linked to one or more PEG molecules are an exemplary embodiment of the invention. In another example, the heterologous molecule is a therapeutic compound or a visualization agent (e.g., a detectable label), and the antibody is being used to transport such heterologous molecule across the BBB. Examples of heterologous molecules include, but are not limited to, a chemical compound, a peptide, a polymer, a lipid, a nucleic acid, and a protein.

The antibody herein may be a “glycosylation variant” such that any carbohydrate attached to the Fc region, if present, is altered, either modified in presence/absence, or modified in type. For example, antibodies with a mature carbohydrate structure that lacks fucose attached to an Fc region of the antibody are described in US Pat Appl No US 2003/0157108 (Presta, L.). See also US 2004/0093621 (Kyowa Hakko Kogyo Co., Ltd). Antibodies with a bisecting N-acetylglucosamine (GlcNAc) in the carbohydrate attached to an Fc region of the antibody are referenced in WO 2003/011878, Jean-Mairet et al. and U.S. Pat. No. 6,602,684, Umana et al. Antibodies with at least one galactose residue in the oligosaccharide attached to an Fc region of the antibody are reported in WO 1997/30087, Patel et al. See, also, WO 1998/58964 (Raju, S.) and WO 1999/22764 (Raju, S.) concerning antibodies with altered carbohydrate attached to the Fc region thereof. See also US 2005/0123546 (Umana et al.) describing antibodies with modified glycosylation. Mutation of the consensus glycosylation sequence in the Fc region (Asn-X-Ser/Thr at positions 297-299, where X cannot be proline), for example by mutating the Asn of this sequence to any other amino acid, by placing a Pro at position 298, or by modifying position 299 to any amino acid other than Ser or Thr should abrogate glycosylation at that position (see, e.g., Fares Al-Ejeh et al., Clin. Cancer Res. (2007) 13:5519s-5527s; Imperiali and Shannon, Biochemistry (1991) 30(18): 4374-4380; Katsuri, Biochem J. (1997) 323(Pt 2): 415-419; Shakin-Eshleman et al., J. Biol. Chem. (1996) 271: 6363-6366).

The term “hypervariable region” or “HVR” as used herein refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs: three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

In some embodiments, HVR residues comprise those identified by the SEQ ID NOs in Table B, below (each column is a separate clone).

### Table B: HVR Sequences

TABLE B
HVR Sequences

| | BSG-A | BSG-B | BSG-C | BSG-D | BSG-E | GLUT1 |
|---|---|---|---|---|---|---|
| LC CDR1 | 3 | 19 | 35 | 51 | 67 | 83 |
| LC CDR2 | 4 | 20 | 36 | 52 | 68 | 84 |
| LC CDR3 | 5 | 21 | 37 | 53 | 69 | 85 |
| HC CDR1 | 6 | 22 | 38 | 54 | 70 | 86 |
| HC CDR2 | 7 | 23 | 39 | 55 | 71 | 87 |
| HC CDR3 | 8 | 24 | 40 | 56 | 72 | 88 |

“LC”, light chain; “HC”, heavy chain; “BSG”, basigin

“LC”, light chain; “HC”, heavy chain; “BSG”, basigin

Unless otherwise indicated, HVR residues and other residues in the variable domain (e.g., FR residues) are numbered herein according to Kabat et al., supra.

An “immunoconjugate” is an antibody conjugated to one or more heterologous molecule(s), including but not limited to a label or cytotoxic agent. Optionally such conjugation is via a linker.

A “linker” as used herein is a structure that covalently or non-covalently connects an antibody to heterologous molecule. In certain embodiments, a linker is a peptide. In other embodiments, a linker is a chemical linker.

A “label” is a marker coupled with the antibody herein and used for detection or imaging. Examples of such labels include: radiolabel, a fluorophore, a chromophore, or an affinity tag. In one embodiment, the label is a radiolabel used for medical imaging, for example tc99m or 1123, or a spin label for nuclear magnetic resonance (NMR) imaging (also known as magnetic resonance imaging, or MRI), for example but not limited to: iodine-123, iodine-131, indium-111, fluorine-19, carbon-13, nitrogen-15, oxygen-17, gadolinium, manganese, and iron.

An “isolated” antibody is one which has been separated from a component of its natural environment. In some embodiments, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC). For review of methods for assessment of antibody purity, see, e.g., Flatman et al., J. Chromatogr. B 848:79-87 (2007).

The term “BACE1,” as used herein, refers to any native beta-secretase 1 (also called β-site amyloid precursor protein cleaving enzyme 1, membrane-associated aspartic protease 2, memapsin 2, aspartyl protease 2 or Asp2) from any vertebrate source, including mammals such as primates (e.g. humans) and rodents (e.g., mice and rats), unless otherwise indicated. The term encompasses “full-length,” unprocessed BACE1 as well as any form of BACE1 which results from processing in the cell. The term also encompasses naturally occurring variants of BACE1, e.g., splice variants or allelic variants. The amino acid sequence of an exemplary BACE1 polypeptide is shown in SEQ ID NO: 111 below, and is the sequence for human BACE1, isoform A as reported in Vassar et al., Science 286:735-741 (1999), which is incorporated herein by reference in its entirety:

MAQALPWLLLWMGAGVLPAHGTQHGIRLPLRSGLGGAPLGLRLPRETDEE PEEPGRRGSFVEMVDNLRGKSGQGYYVEMTVGSPPQTLNILVDTGSSNFAVGAAP HPFLHRYYQRQLSSTYRDLRKGVYVPYTQGKWEGELGTDLVSIPHGPNVTVRANI AAITESDKFFINGSNWEGILGLAYAEIARPDDSLEPFFDSLVKQTHVPNLFSLQLCGA GFPLNQSEVLASVGGSMIIGGIDHSLYTGSLWYTPIRREWYYEVIIVRVEINGQDLK MDCKEYNYDKSIVDSGTTNLRLPKKVFEAAVKSIKAASSTEKFPDGFWLGEQLVC WQAGTTPWNIFPVISLYLMGEVTNQSFRITILPQQYLRPVEDVATSQDDCYKFAISQ SSTGTVMGAVIMEGFYVVFDRARKRIGFAVSACHVHDEFRTAAVEGPFVTLDMED CGYNIPQTDESTLMTIAYVMAAICALFMLPLCLMVCQWCCLRCLRQQHDDFADDI SLLK (SEQ ID NO: 111).

Several other isoforms of human BACE1 exist including isoforms B, C and D. See UniProtKB/Swiss-Prot Entry P56817, which is incorporated herein by reference in its entirety. Isoform B differs from isoform A in that it is missing amino acids 190-214 (i.e. deletion of amino acids 190-214 of SEQ ID NO: 111). Isoform C and differs from isoform A in that it is missing amino acids 146-189 (i.e. deletion of amino acids 146-189 of (SEQ ID NO: 111). Isoform D differs from isoform A in that it is missing amino acids 146-189 and 190-214 (i.e. deletion of amino acids 146-189 and 190-214 of SEQ ID NO: 111).

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (Kd). Affinity can be measured by common methods known in the art, including those described herein. Specific illustrative and exemplary embodiments for measuring binding affinity are described in the following.

An “affinity matured” antibody refers to an antibody with one or more alterations in one or more hypervariable regions (HVRs), compared to a parent antibody which does not possess such alterations, such alterations resulting in an improvement in the affinity of the antibody for antigen.

The terms “anti-Bsg antibody,” “anti-basigin antibody,” “an antibody that binds to basigin,” and “an antibody that binds to Bsg” refer to an antibody that is capable of binding basigin without impairing the binding of basigin to one or more of its native ligands. In one embodiment, the extent of binding of an anti-Bsg antibody to an unrelated, non-Bsg protein is less than about 10% of the binding of the antibody to Bsg as measured, e.g., by a radioimmunoassay (RIA). In certain embodiments, an antibody that binds to Bsg has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g., from 10 M to 10⁻¹³ M, e.g., from 10⁻⁹ M to 10⁻¹³ M). In certain embodiments, an anti-Bsg antibody binds to an epitope of Bsg that is conserved among Bsg from different species (e.g., human Bsg and murine Bsg). In certain embodiments, an anti-Bsg antibody binds to an epitope of Bsg that is conserved among different Bsg isoforms (e.g., two or more human Bsg isoforms).

The terms “anti-Glut1 antibody” and “an antibody that binds to Glut1” refer to an antibody that is capable of binding Glut1 without impairing the binding of Glut1 to one or more of its native ligands. In one embodiment, the extent of binding of an anti-Glut1 antibody to an unrelated, non-Glut1 protein is less than about 10% of the binding of the antibody to Glut1 as measured, e.g., by a radioimmunoassay (RIA). In certain embodiments, an antibody that binds to Glut1 has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g., from 10⁻⁸ M to 10⁻¹³ M, e.g., from 10⁻⁹ M to 10⁻¹³ M). In certain embodiments, an anti-Glut1 antibody binds to an epitope of Glut1 that is conserved among Glut1 from different species (e.g., human Glut1 and murine Glut1).

According to the present invention, a “low affinity” anti-BBB-R (e.g. anti-CD98hc, anti-Bsg, or anti-Glut1) antibody can be identified by various assays for measuring antibody affinity, for example and without limitation, the Scatchard assay and surface plasmon resonance technique (e.g. using BIACORE®). According to one embodiment of the invention, the antibody has an affinity for the BBB-R antigen (e.g. for CD98hc, Bsg, or Glutl) from about 5 nM, or from about 20 nM, or from about 100 nM, to about 10 μM, or to about 1 μM, or to about 500 nM. Thus, the affinity may be in the range from about 5 nM to about 10 μM, or in the range from about 20 nM to about 1 μM, or in the range from about 100 nM to about 500 nM, e.g. as measured by Scatchard analysis or BIACORE®.

An “isolated nucleic acid” refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

As used herein, an “isolated nucleic acid encoding an antibody” (e.g. an anti-CD98hc, anti-Bsg, or anti-Glut1 antibody) refers to one or more nucleic acid molecules encoding antibody heavy and light chains (or fragments thereof), including such nucleic acid molecule(s) in a single vector or separate vectors, and such nucleic acid molecule(s) present at one or more locations in a host cell.

A “naked antibody” refers to an antibody that is not conjugated to a heterologous moiety (e.g., a cytotoxic moiety or radiolabel). The naked antibody may be present in a pharmaceutical formulation.

Antibody “effector functions” refer to those biological activities of an antibody that result in activation of the immune system other than activation of the complement pathway. Such activities are largely found in the Fc region (a native sequence Fc region or amino acid sequence variant Fc region) of an antibody. Examples of antibody effector functions include, for example, Fc receptor binding and antibody-dependent cell-mediated cytotoxicity (ADCC). In one embodiment, the antibody herein essentially lacks effector function. In another embodiment, the antibody herein retains minimal effector function. Methods of modifying or eliminating effector function are well-known in the art and include, but are not limited to, eliminating all or a portion of the Fc region responsible for the effector function (e.g., using an antibody or antibody fragment in a format lacking all or a portion of the Fc region such as, but not limited to, a Fab fragment, a single-chain antibody, and the like as described herein and as known in the art); modifying the Fc region at one or more amino acid positions to eliminate effector function (Fc binding-impacting: positions 234, 235, 238, 239, 248, 249, 252, 254, 256, 265, 268, 269, 270, 272, 278, 289, 292, 293, 294, 295, 296, 297, 298, 301, 303, 311, 322, 324, 327, 329, 333, 335, 338, 340, 373, 376, 382, 388, 389, 414, 416, 419, 434, 435, 436, 437, 438, and 439); and modifying the glycosylation of the antibody (including, but not limited to, producing the antibody in an environment that does not permit wild-type mammalian glycosylation, removing one or more carbohydrate groups from an already-glycosylated antibody, and modifying the antibody at one or more amino acid positions to eliminate the ability of the antibody to be glycosylated at those positions (including, but not limited to N297G and N297A and D265A).

Antibody “complement activation” functions or properties of an antibody that enable or trigger “activation of the complement pathway” are used interchangeably, and refer to those biological activities of an antibody that engage or stimulate the complement pathway of the immune system in a subject. Such activities include, e.g., Clq binding and complement dependent cytotoxicity (CDC), and may be mediated by both the Fc portion and the non-Fc portion of the antibody. Methods of modifying or eliminating complement activation function are well-known in the art and include, but are not limited to, eliminating all or a portion of the Fc region responsible for complement activation (e.g., using an antibody or antibody fragment in a format lacking all or a portion of the Fc region such as, but not limited to, a Fab fragment, a single-chain antibody, and the like as described herein and as known in the art, or modifying the Fc region at one or more amino acid positions to eliminate or lessen interactions with complement components or the ability to activate complement components, such as positions 270, 322, 329 and 321, known to be involved in C1q binding), and modifying or eliminating a portion of the non-Fc region responsible for complement activation (e.g., modifying the CH1 region at position 132 (see, e.g., Vidarte et al., (2001) J. Biol. Chem. 276(41): 38217-38223)).

Depending on the amino acid sequence of the constant domain of their heavy chains, full length antibodies can be assigned to different “classes”. There are five major classes of full length antibodies: IgA, IgD, IgE, IgG, and IgM, and several of these may be further divided into “subclasses” (isotypes), e.g., IgG1, IgG2, IgG3, IgG4, IgA, and IgA2. The heavy-chain constant domains that correspond to the different classes of antibodies are called alpha, delta, epsilon, gamma, and mu, respectively. The subunit structures and three-dimensional configurations of different classes of immunoglobulins are well known in the art.

The term “recombinant antibody”, as used herein, refers to an antibody (e.g. a chimeric, humanized, or human antibody or antigen-binding fragment thereof) that is expressed by a recombinant host cell comprising nucleic acid encoding the antibody.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable region (VH), also called a variable heavy domain or a heavy chain variable domain, followed by three constant domains (CH1, CH2, and CH3). Similarly, from N- to C-terminus, each light chain has a variable region (VL), also called a variable light domain or a light chain variable domain, followed by a constant light (CL) domain. The light chain of an antibody may be assigned to one of two types, called kappa (κ) and lambda (λ), based on the amino acid sequence of its constant domain.

The term “package insert” is used to refer to instructions customarily included in commercial packages of therapeutic products, that contain information about the indications, usage, dosage, administration, combination therapy, contraindications and/or warnings concerning the use of such therapeutic products.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “pharmaceutical formulation” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical formulation, other than an active ingredient, which is nontoxic to a subject, A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector,” as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors.”

CD98 Heavy Chain

The term “CD98 heavy chain” or “CD98hc” as used herein, refers to any native CD98hc from any vertebrate source, including mammals such as primates (e.g., humans) and rodents (e.g., mice and rats), unless otherwise indicated. CD98hc is also known by the names, inter alia, SLC3A2, 4F2, 4F2hc, Mdu1, Ly10, Mdv1, Frp1, Mgp2, Mgp2hc, NACAE, 4T2, 4T2hc, and TROP4. The term CD98hc encompasses “full-length,” unprocessed CD98hc as well as any form of CD98hc which results from processing in the cell. The term also encompasses naturally occurring variants of CD98hc, e.g., splice variants or allelic variants. CD98hc is 80 kDa type II transmembrane protein and pairs with 6 different light chain (“lc”) members or “binding partners” of the L-type amino transporter family of about 40 kDa (LAT1, LAT2, y+LAT1, y+LAT2, xCT, Asc) by a disulfide bond to form a heterodimeric complex (see, Yanagida, et al. Biochim. Biophys. Acta 1514:291-302(2001); Torrents et al. J. Biol. Chem. 273:32437-32445(1998); Broeer et al. Biochem. J. 349:787-795(2000); Broeer et al. Biochem. J. 355:725-731(2001); Sato et al. Antioxid Redox Signal. 2000 Winter; 2(4):665-71). Thus, as used herein, “CD98 heterodimeric complex” refers to protein complexes comprising the CD98 heavy chain (e.g., LAT1/CD98hc, LAT2/CD98hc, y+LAT1/CD98hc, y+LAT2/CD98hc, xCT/CD98hc, and/or Asc/CD98hc). The CD98 heterodimeric complex functions as an amino acid transporter. CD98hc is required for the surface expression and basolateral localization of the amino acid transporter complex in polarized epithelial cells (Okubo et al. J Neurooncol (2010) 99:217-225; Palacin and Kanai. Pflügers Archiv; February 2004, 447(5):490-494). CD98hc also interacts with beta 1 integrins and regulates their activation through the cytoplasmic domains and transmembrane regions. Studies suggest that overexpression of CD98hc may contribute to cell growth and survival by regulating integrin signaling, and therefore may play an important role in tumorigenesis. Studies have shown that CD98hc expression is tightly linked to cell proliferation, and certain antibodies against CD98hc can inhibit cell growth or induce apoptosis in specific cell types (Yagita H. et al. (1986) Cancer Res. 46:1478-1484; Warren A. P., et al. (1996) Blood 87:3676-3687).

The structure of the ectodomain of human CD98hc has been solved using monoclinic (Protein Data Bank code 2DH2) and orthorhombic (Protein Data Bank code 2DH3) crystal forms at 2.1 and 2.8 Å, respectively. It is composed of a (betaalpha)(8) barrel and an antiparallel beta(8) sandwich related to bacterial alpha-glycosidases, although lacking key catalytic residues and consequently catalytic activity. 2DH3 is a dimer with Zn(2+) coordination at the interface. CD98hc has no significant hydrophobic patches at the surface. The CD98hc monomer and homodimer have a polarized charged surface. The N terminus of the solved structure, including the position of Cys109 residue located four residues apart from the transmembrane domain, is adjacent to the positive face of the ectodomain. This location of the N terminus and the Cys(109)-intervening di sulfide bridge imposes space restrictions sufficient to support a model for electrostatic interaction of the CD98hc ectodomain with membrane phospholipids (see, Fort et al. J Biol Chem. 2007 Oct. 26; 282(43):31444-52). Cys¹⁰⁹ is near the transmembrane domain of CD98hc and results in a disulfide bridge with a cysteine in an extracellular loop of the light chain between transmembrane domains 3 and 4. Mutation of Cys¹⁰⁹ and Cys³³⁰ disrupted the covalent association with the light chain but did not impair interactions with or effects on integrins. However, it was reported that a C109S mutant does still support the surface expression of the light chain and displays transport characteristics at a reduced rate (Pfeiffer R., et al. (1998) FEBS Lett. 439:157-162).

In some embodiments, the CD98hc disclosed herein is human CD98hc (“hCD98hc”) comprising the amino acid sequence as set forth in SEQ ID NO: 97, 99, 101, or 103, which correspond to GenBank® Accession Nos. NP_001012680.1 (isoform b), NP_002385.3 (isoform c), NP_001012682.1 (e), and NP_001013269.1 (isoform f), respectively. Isoforms b, c, e, and f are encoded by the nucleic acids having GenBank® Accession Nos.: NM_001012662.2 (SEQ ID NO: 98), NM_002394.5 (SEQ ID NO: 100), NM_001012664.2 (SEQ ID NO: 102), and NM_001013251.2 (SEQ ID NO: 104), respectively. In another embodiment, the CD98hc disclosed herein is primate CD98hc (“pCD98hc”) comprising the amino acid sequence as set forth in GenBank® Accession No.: NP_001272171.1 (SEQ ID NO: 109), which is encoded by the nucleic acid sequence as set forth in GenBank® Accession No.: NM_001285242.1 (SEQ ID NO: 110). In another embodiment, the CD98hc disclosed herein is murine CD98hc (“mCD98hc”) comprising the amino acid sequence as set forth in GenBank® Accession No.: NP_001154885.1 (isoform a) (SEQ ID NO: 105), which is encoded by the nucleic acid sequence as set forth in GenBank® Accession No.: NM_001161413.1 (SEQ ID NO: 106). In another embodiment, the murine CD98hc disclosed herein comprises the amino acid sequence as set forth in GenBank® Accession No.: NP_032603.3 (isoform b) (SEQ ID NO: 107), which is encoded by the nucleic acid sequence as set forth in GenBank® Accession No.: NM_008577.4 (SEQ ID NO: 108).

In certain embodiments, the CD98hc is glycosylated. In certain embodiments, the CD98hc is phosphorylated.

In certain embodiments, the transmembrane domain of CD98hc consists of amino acid residues 185-205 of SEQ ID NO: 97 (isoform b), the extracellular domain of hCD98hc consists of amino acid residues 206-630 of SEQ ID NO: 97 (isoform b), and the cytoplasmic domain of CD98hc consists of amino acid residues 102-184 of SEQ ID NO: 97 (isoform b). In certain embodiments, the extracellular domain of CD98hc consists of amino acid residues 105-529 of SEQ ID NO: 103 (isoform f).

The terms “anti-CD98hc antibody” and “an antibody that binds to CD98hc” refer to an antibody that is capable of binding CD98hc. In certain embodiments, the extent of binding of an anti-CD98hc antibody to an unrelated, non-CD98hc protein is less than about 10% of the binding of the antibody to CD98hc as measured, e.g., by a radioimmunoassay (RIA).

Basigin

The term “Bsg,” as used herein, refers to any native basigin (also known as CD147 or EMMPRIN) from any vertebrate source, including mammals such as primates (e.g. humans) and rodents (e.g., mice and rats), unless otherwise indicated. Other synonyms for Bsg include 5F7, OK, TCSF, HT7, 5A11, gp42, neurothelin, OX-47, and HAb18. The term encompasses “full-length,” unprocessed Bsg as well as any form of Bsg that results from processing in the cell. The term also encompasses naturally occurring variants of Bsg, e.g., splice variants or allelic variants. Examples of naturally occurring variants include human Bsg1 (176 amino acids), Bsg2 (269 amino acids), Bsg3 (385 amino acids), and Bsg4 (205 amino acids), of which Bsg2 is the predominant form found in humans. The amino acid sequence of an exemplary human Bsg2 is shown in SEQ ID NO: 112. The amino acid sequence of an exemplary murine Bsg is shown in SEQ ID NO: 113.

Glut1

The term “Glut1,” as used herein, refers to any native glucose transporter 1 from any vertebrate source, including mammals such as primates (e.g. humans) and rodents (e.g., mice and rats), unless otherwise indicated. Other synonyms for Glut1 include glucose transporter type 1, solute carrier family 2, member 1, SLC2A1, HTLVR, and human T-cell leukemia virus receptor. The term encompasses “full-length,” unprocessed Glut1 as well as any form of Glut1 that results from processing in the cell. The term also encompasses naturally occurring variants of Glut1, e.g., splice variants or allelic variants. The amino acid sequence of an exemplary human Glut1 is shown in SEQ ID NO: 114.

### II. Compositions and Methods

In certain aspects, the present invention provides compositions and/or methods for transporting an agent across the blood-brain barrier. In some aspects, an agent is transported across the blood-brain barrier using an antibody against CD98hc, Glut1 or basigin. In some embodiments, an anti-Basigin/BACE1 antibody is provided. In some embodiments, an anti-Glut1/BACE1 antibody is provided. In certain embodiment, an anti-CD98hc/BACE1 antibody for use in a method of transporting an agent across the blood-brain barrier is provided. In certain embodiments, antibodies contemplated herein bind to human and/or primate CD98hc, basigin, or Glut1. Antibodies of the invention are also useful, e.g., for the treatment of diseases or disorders affecting the CNS (e.g., brain).

A. Production of Anti-BBB-R Antibodies and Conjugates Thereof

The methods and articles of manufacture of the present invention use, or incorporate, an antibody that binds to BBB-R. The BBB-R antigen to be used for production of, or screening for, antibodies may be, e.g., a soluble form of or a portion thereof (e.g. the extracellular domain), containing the desired epitope. Alternatively, or additionally, cells expressing BBB-R at their cell surface can be used to generate, or screen for, antibodies. Other forms of BBB-R useful for generating antibodies will be apparent to those skilled in the art. Examples of BBB-Rs herein include CD98hc, Glut1 and Basigin.

In one aspect, the invention provides a method of making an antibody useful for transporting an agent (e.g., a neurological disorder drug or imaging agent) across the blood-brain barrier comprising selecting an antibody from a panel of antibodies against a BBB-R because it does not inhibit cell growth. In another aspect, the invention provides a method of making an antibody useful for transporting an agent (e.g., a neurological disorder drug or imaging agent) across the blood-brain barrier comprising selecting an antibody from a panel of antibodies against a BBB-R because it does not induce apoptosis. In another aspect, the invention provides a method of making an antibody useful for transporting an agent (e.g., a neurological disorder drug or imaging agent) across the blood-brain barrier comprising selecting an antibody from a panel of antibodies against a BBB-R because it does not inhibit one or more known functions of the BBB-R. In another aspect, the invention provides a method of making an antibody useful for transporting an agent (e.g., a neurological disorder drug or imaging agent) across the blood-brain barrier comprising selecting an antibody from a panel of antibodies against a BBB-R because it does not inhibit one or more of the known functions of the BBB-R. In a specific embodiment, the antibody binds to CD98hc and does not inhibit amino acid transport by the CD98 heterodimeric complex. In vitro assay which may be used to detect amino acid transport by CD98hc (e.g., in a heterodimeric complex with a CD98 light chain (e.g., LAT1, LAT2, y+LAT1, y+LAT2, xCT, and asc-1) are known and described in the art. See, e.g., Fenczik, C. A et al. (2001) J. Biol. Chem. 276, 8746-8752; see also, US 2013/0052197. In another aspect, the invention provides a method of making an antibody useful for transporting an agent (e.g., a neurological disorder drug or imaging agent) across the blood-brain barrier comprising selecting an antibody from a panel of antibodies against a BBB-R because it does not inhibit interaction of the BBB-R with one or more of its binding partners (e.g. does not inhibit the interaction of CD98hc with a light chain binding partner (e.g., LAT1, LAT2, y+LAT1, y+LAT2, xCT, and Asc-1).

In another aspect, the invention provides a method of making an antibody useful for transporting an agent (e.g., a neurological disorder drug or imaging agent) across the blood-brain barrier comprising selecting an antibody from a panel of antibodies against a BBB-R because binding of the BBB-R to one or more of its native ligands in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of binding in the absence of the anti-BBB-R antibody. In a specific embodiment, the BBB-R is CD98hc. In another specific embodiment, the BBB-R is Glut1. In another specific embodiment, the BBB-R is Basigin. Methods for determining binding to a native ligand are known in the art (e.g., immunoprecipitation assays, ELISA, etc.).

In another aspect, the invention provides a method of making an antibody useful for transporting an agent (e.g., a neurological disorder drug or imaging agent) across the blood-brain barrier comprising selecting an antibody from a panel of antibodies against a BBB-R because transport of one or more of the native ligands of the BBB-R across the blood-brain barrier in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of transport in the absence of the antibody. In another specific embodiment, the BBB-R is CD98hc. In another specific embodiment, the BBB-R is Glut1. In another specific embodiment, the BBB-R is Basigin.

In another aspect, the invention provides a method of making an anti-CD98hc antibody useful for transporting an agent (e.g., a neurological disorder drug or imaging agent) across the blood-brain barrier comprising selecting an antibody from a panel of antibodies against CD98hc because binding of CD98hc to its light chain binding partner (e.g., LAT1, LAT2, y+LAT1, y+LAT2, xCT, or Asc-1) in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of binding in the absence of the antibody. In a specific embodiment, the amount of binding of CD98hc to its light chain binding partner is at least 80%. In a specific embodiment, the amount of binding of CD98hc to its light chain binding partner is at least 90%. In a specific embodiment, the amount of binding of CD98hc to its light chain binding partner is at least 95%.

In another aspect, the invention provides a method of making an anti-CD98hc antibody useful for transporting an agent (e.g., a neurological disorder drug or imaging agent) across the blood-brain barrier comprising selecting an antibody from a panel of antibodies against CD98hc because the amount of amino acid transport across the BBB in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of amino acid transport across the BBB in the absence of the antibody. In a specific embodiment, the amount of amino acid transport across the BBB is at least 80% of the amount of amino acid transport across the BBB in the absence of the antibody. In another specific embodiment, the amount of amino acid transport across the BBB is at least 90% of the amount of amino acid transport across the BBB in the absence of the antibody. In another specific embodiment, the amount of binding of CD98hc to its light chain binding partner is at least 95% of the amount of amino acid transport across the BBB in the absence of the antibody. In another specific embodiment, the amount of binding of CD98hc to its light chain binding partner is at least 99% of the amount of amino acid transport across the BBB in the absence of the antibody. In another specific embodiment, the amount of binding of CD98hc to its light chain binding partner is 100% of the amount of amino acid transport across the BBB in the absence of the antibody.

In another aspect, the invention provides a method of making an antibody useful for transporting an agent (e.g., a neurological disorder drug or imaging agent) across the blood-brain barrier comprising selecting an antibody from a panel of antibodies against a blood-brain barrier receptor (BBB-R) because it has an affinity for the BBB-R which is in the range from about 5 nM, or from about 20 nM, or from about 100 nM, to about 10 μM, or to about 1 μM, or to about 500 mM. Thus, the affinity may be in the range from about 5 nM to about 10 μM or in the range from about 20 nM to about 1 μM, or in the range from about 100 nM to about 500 nM, e.g. as measured by Scatchard analysis or BIACORE®. As will be understood by one of ordinary skill in the art, conjugating a heterologous molecule/compound to an antibody will often decrease the affinity of the antibody for its target due, e.g., to steric hindrance or even to elimination of one binding arm if the antibody is made multispecific with one or more arms binding to a different antigen than the antibody's original target.

B. Therapeutic Methods and Compositions

Anti-CD98hc, anti-Bsg, and anti-Glut1 antibodies, e.g., as described herein, may be 5 used in therapeutic methods. For example, an anti-CD98hc, anti-Bsg, or anti-Glut1 antibody is useful as a medicament. In some aspects, an anti-CD98hc, anti-Bsg, or anti-Glut1 antibody is useful for treating a neurological disease or disorder, e.g., by delivering a therapeutic agent (e.g., a therapeutic drug, e.g., antibody) to a CNS site (e.g., brain). Non-limiting examples of neurological diseases or disorders encompassed by the uses and methods disclosed herein include, e.g., Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer (e.g., brain cancer, e.g., glioma, e.g., glioblastoma multiforme), and traumatic brain injury.

In certain embodiments, the invention provides a method of treating an individual having a neurological disease or disorder, wherein the method includes administering to the individual an effective amount of an anti-CD98hc, anti-Bsg, or anti-Glut1 antibody, wherein the anti-CD98hc, anti-Bsg, or anti-Glut1 antibody delivers a therapeutic agent across the blood-brain barrier. In certain embodiments, an effective amount of the anti-CD98hc, anti-Bsg, or anti-Glut1 antibody is an amount that is effective for transporting a therapeutic agent across the BBB. In one such embodiment, the method further includes administering to the individual an effective amount of at least one additional therapeutic agent, e.g., as described below. In some embodiments, the subject has not been diagnosed with cancer. In some embodiments, the subject has not been diagnosed with brain cancer. In some embodiments, the subject does not have cancer. In some embodiments, the subject does not have brain cancer.

In certain embodiments, the invention provides an anti-CD98hc, anti-Bsg, or anti-Glut1 antibody for use in transporting an agent across the BBB. In certain embodiments, the invention provides an anti-CD98hc, anti-Bsg, or anti-Glut1 antibody for use in a method of transporting an agent across the BBB in an individual comprising administering to the individual an effective amount of the anti-CD98hc, anti-Bsg, or anti-Glut1 antibody to transport the agent across the BBB. By way of example, and without limitation, an anti-CD98hc, anti-Bsg, or anti-Glut1 antibody herein may be a multispecific antibody (e.g., a bispecific antibody), and can comprise a therapeutic arm which is specific for a brain antigen of interest (e.g., a target). Without intending to be limited by any one particular theory or mechanism of action, it is expected that the anti-CD98hc, anti-Bsg, or anti-Glutl antibody portion of the multispecific antibody binds to the target receptor on the BBB and is transported to the abluminal side of the BBB. The therapeutic arm of the antibody (e.g., the portion specific for a brain antigen) is then able to bind to the target brain antigen.

In a specific example, a CD98hc/BACE1 bispecific antibody binds to CD98hc on the BBB, is then transported to the abluminal side of the BBB, and then the BACE1 antibody portion binds to BACE1 in the brain. In another specific example, a CD98hc/BACE1 bispecific antibody binds to CD98hc on the BBB, is then transported to the abluminal side of the BBB via the CD98 amino acid transporter, and then the BACE1 antibody portion binds to BACE1. This would be useful, e.g., for inhibiting BACE1, which would lead to a decrease in soluble Abeta levels.

In another specific example, a Basigin/BACE1 bispecific antibody binds to basigin on the BBB, is then transported to the abluminal side of the BBB via basigin, and then the BACE1 antibody portion binds to BACE1. This would be useful, e.g., for inhibiting BACE1, which would lead to a decrease in soluble Abeta levels.

In another specific example, a Glut1/BACE1 bispecific antibody binds to Glut1 on the BBB, is then transported to the abluminal side of the BBB via Glut1, and then the BACE1 antibody portion binds to BACE1. This would be useful, e.g., for inhibiting BACE1, which would lead to a decrease in soluble Abeta levels. In some embodiments, the Glut1-specific portion of the antibody does not inhibit glucose transport to the brain by Glut1.

In a further aspect, the invention provides for the use of an anti-CD98hc, anti-Bsg, or anti-Glut1 antibody in the manufacture or preparation of a medicament. In one embodiment, the medicament is for treatment of a neurological disease or disorder (e.g., Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer (e.g., brain cancer, e.g., glioma, e.g., glioblastoma multiforme), and traumatic brain injury). In a further embodiment, the medicament is for use in a method of treating a neurological disease or disorder comprising administering to an individual having the neurological disease or disorder an effective amount of the medicament. In one such embodiment, the method further comprises administering to the individual an effective amount of at least one additional therapeutic agent, e.g., as described below. In a further embodiment, the medicament is for, e.g., decreasing levels of a protein such as BACE1, Abeta, EGFR, HER2, Tau, apolipoprotein (e.g., ApoE4), alpha-synuclein, CD20, huntingtin, PrP, LRRK2, parkin, presenilin 1, presenilin 2, gamma secretase, DR6, APP, p75NTR, and caspase 6. In a further embodiment, the medicament is for use in a method of transporting an agent across the BBB in an individual comprising administering to the individual an amount effective of the medicament to transport the agent across the BBB.

In some aspects of the above-described therapeutic methods and uses, the anti-CD98hc antibody used in the method does not impair the normal and/or reported functions of CD98hc (e.g., amino acid transport). In some aspect, the anti-CD98hc antibody does not impair binding of CD98hc to one or more of its native ligands. In some aspects, the anti-CD98hc antibody does not impair binding of a CD98 heterodimeric complex (comprised of CD98hc and a light chain binding partner (e.g., LAT1, LAT2, y+LAT1, y+LAT2, xCT, and Asc-1)) to one or more native ligands of the heterodimeric complex. In some aspects, anti-CD98hc antibody does not inhibit the pairing of CD98hc with its light chain binding partner (e.g., LAT1, LAT2, y+LAT1, y+LAT2, xCT, and Asc-1).

In some aspects of the therapeutic methods, binding of the CD98hc and/or of a CD98 heterodimeric complex to one or more of its native ligands in the presence of the CD98hc antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of binding in the absence of the anti-CD98hc antibody.

In some aspects of the therapeutic methods, transport of one or more of the native ligands of a CD98 heterodimeric complex across the blood-brain barrier in the presence of a CD98hc antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%), of the amount of transport in the absence of the anti-CD98hc antibody.

In some aspects of the therapeutic methods, the anti-CD98hc antibody does not induce cell death and/or apoptosis. In another aspect, the anti-CD98hc antibody does not inhibit cell proliferation. In another aspect, the anti-CD98hc antibody does not inhibit cell division. In another aspect, the anti-CD98hc antibody does not inhibit cell adhesion. In some aspects, the anti-CD98hc antibody does not induce cell death or apoptosis, and does not inhibit cell proliferation. In some aspects, the anti-CD98hc antibody does not induce cell death or apoptosis, and does not inhibit cell proliferation, cell division, or cell adhesion.

In some aspects of the therapeutic methods, the anti-CD98hc antibody binds to a region in the extracellular domain of CD98hc (e.g., binds to an epitope in the region spanning amino acid residues 105 to 529 of SEQ ID NO: 103). In some aspects, the anti-CD98hc antibody binds to an epitope that does not include the extracellular cysteine Cysl09. In some aspects, the anti-CD98hc antibody binds to an epitope that does not include the extracellular cysteine Cys210. In some embodiments, the anti-CD98hc antibody binds to an epitope that does not include the extracellular cysteine Cys330 of the canonical 630 amino acid CD98hc sequence (isoform c, SEQ ID NO: 99).

In some aspects, the anti-CD98hc antibody binds to CD98hc with sufficient affinity such that the antibody is useful for transporting therapeutic agents across the BBB. In certain embodiments, an anti-CD98hc antibody for use in the methods has a dissociation constant (Kd) of ≦1 M, ≦100 nM, ≦10 nM, ≦1 nM, ≦0.1 nM, ≦0.01 nM, or ≦0.001 nM (e.g. 10⁻⁸ M or less, e.g., from 10⁻⁸ M to 10⁻¹³ M, e.g., from 10⁻⁹ M to 10⁻¹³ M). In certain embodiments, an anti-CD98hc antibody binds to an epitope of CD98hc that is conserved among CD98hc from different species.

In any of the above aspects, the anti-CD98hc antibody can be a humanized antibody.

In some aspects of the therapeutic methods, transport of one or more of the native ligands of basigin across the blood-brain barrier in the presence of an anti-Bsg antibody disclosed herein is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of transport in the absence of the anti-Bsg antibody.

In any of the above embodiments, an anti-Bsg antibody can be a humanized antibody.

In some aspects of the therapeutic methods, transport of one or more of the native ligands of Glut1 across the blood-brain barrier in the presence of an anti-Glut1 antibody disclosed herein is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of transport in the absence of the anti-Glut1 antibody.

In any of the above embodiments, an anti-Glut1 antibody can be a humanized antibody.

As disclosed above, the methods disclosed herein include methods for treating diseases and disorders of the brain and/or CNS.

For example, and without limitation, neuropathy disorders may be treated according to the therapeutic methods and with the compositions disclosed herein. Neuropathy disorders are diseases or abnormalities of the nervous system characterized by inappropriate or uncontrolled nerve signaling or lack thereof, and include, but are not limited to, chronic pain (including nociceptive pain), pain caused by an injury to body tissues, including cancer-related pain, neuropathic pain (pain caused by abnormalities in the nerves, spinal cord, or brain), and psychogenic pain (entirely or mostly related to a psychological disorder), headache, migraine, neuropathy, and symptoms and syndromes often accompanying such neuropathy disorders such as vertigo or nausea.

For a neuropathy disorder, a neurological drug may be selected that is an analgesic including, but not limited to, a narcotic/opioid analgesic (e.g., morphine, fentanyl, hydrocodone, meperidine, methadone, oxymorphone, pentazocine, propoxyphene, tramadol, codeine and oxycodone), a nonsteroidal anti-inflammatory drug (NSAID) (e.g., ibuprofen, naproxen, diclofenac, diflunisal, etodolac, fenoprofen, flurbiprofen, indomethacin, ketorolac, mefenamic acid, meloxicam, nabumetone, oxaprozin, piroxicam, sulindac, and tolmetin), a corticosteroid (e.g., cortisone, prednisone, prednisolone, dexamethasone, methylprednisolone and triamcinolone), an anti-migraine agent (e.g., sumatriptin, almotriptan, frovatriptan, sumatriptan, rizatriptan, eletriptan, zolmitriptan, dihydroergotamine, eletriptan and ergotamine), acetaminophen, a salicylate (e.g., aspirin, choline salicylate, magnesium salicylate, diflunisal, and salsalate), a anti-convulsant (e.g., carbamazepine, clonazepam, gabapentin, lamotrigine, pregabalin, tiagabine, and topiramate), an anaesthetic (e.g., isoflurane, trichloroethylene, halothane, sevoflurane, benzocaine, chloroprocaine, cocaine, cyclomethycaine, dimethocaine, propoxycaine, procaine, novocaine, proparacaine, tetracaine, articaine, bupivacaine, carticaine, cinchocaine, etidocaine, levobupivacaine, lidocaine, mepivacaine, piperocaine, prilocaine, ropivacaine, trimecaine, saxitoxin and tetrodotoxin), and a cox-2-inhibitor (e.g., celecoxib, rofecoxib, and valdecoxib). For a neuropathy disorder with vertigo involvement, a neurological drug may be selected that is an anti-vertigo agent including, but not limited to, meclizine, diphenhydramine, promethazine and diazepam. For a neuropathy disorder with nausea involvement, a neurological drug may be selected that is an anti-nausea agent including, but not limited to, promethazine, chlorpromazine, prochlorperazine, trimethobenzamide, and metoclopramide.

For example, and without limitation, amyloidoses may be treated according to the therapeutic methods and with the compositions disclosed herein. Amyloidoses are a group of diseases and disorders associated with extracellular proteinaceous deposits in the CNS, including, but not limited to, secondary amyloidosis, age-related amyloidosis, Alzheimer's Disease (AD), mild cognitive impairment (MCI), Lewy body dementia, Down's syndrome, hereditary cerebral hemorrhage with amyloidosis (Dutch type); the Guam Parkinson-Dementia complex, cerebral amyloid angiopathy, Huntington's disease, progressive supranuclear palsy, multiple sclerosis; Creutzfeld Jacob disease, Parkinson's disease, transmissible spongiform encephalopathy, HIV-related dementia, amyotropic lateral sclerosis (ALS), inclusion-body myositis (IBM), and ocular diseases relating to beta-amyloid deposition (e.g., macular degeneration, drusen-related optic neuropathy, and cataract).

For amyloidosis, a neurological drug may be selected that includes, but is not limited to, an antibody or other binding molecule (including, but not limited to a small molecule, a peptide, an aptamer, or other protein binder) that specifically binds to a target selected from: beta secretase, tau, presenilin, amyloid precursor protein or portions thereof, amyloid beta peptide or oligomers or fibrils thereof, death receptor 6 (DR6), receptor for advanced glycation endproducts (RAGE), parkin, and huntingtin; a cholinesterase inhibitor (e.g., galantamine, donepezil, rivastigmine and tacrine); an NMDA receptor antagonist (e.g., memantine), a monoamine depletor (e.g., tetrabenazine); an ergoloid mesylate; an anticholinergic antiparkinsonism agent (e.g., procyclidine, diphenhydramine, trihexylphenidyl, benztropine, biperiden and trihexyphenidyl); a dopaminergic antiparkinsonism agent (e.g., entacapone, selegiline, pramipexole, bromocriptine, rotigotine, selegiline, ropinirole, rasagiline, apomorphine, carbidopa, levodopa, pergolide, tolcapone and amantadine); a tetrabenazine; an anti-inflammatory (including, but not limited to, a nonsteroidal anti-inflammatory drug (e.g., indomethicin and other compounds listed above); a hormone (e.g., estrogen, progesterone and leuprolide); a vitamin (e.g., folate and nicotinamide); a dimebolin; a homotaurine (e.g., 3-aminopropanesulfonic acid; 3APS); a serotonin receptor activity modulator (e.g., xaliproden); an, an interferon, and a glucocorticoid.

For example, and without limitation, cancer may be treated according to the therapeutic methods and with the compositions disclosed herein. Cancers of the CNS are characterized by aberrant proliferation of one or more CNS cell (e.g., a neural cell) and include, but are not limited to, glioma, glioblastoma multiforme, meningioma, astrocytoma, acoustic neuroma, chondroma, oligodendroglioma, medulloblastomas, ganglioglioma, Schwannoma, neurofibroma, neuroblastoma, and extradural, intramedullary or intradural tumors.

For cancer, a neurological drug may be selected (e.g., and conjugated to or co-administered with an anti-CD98hc, Glut1 or Bsg antibody) that is a chemotherapeutic agent. Examples of chemotherapeutic agents include alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphor-amide and trimethylolomelamine; acetogenins (especially bullatacin and bullatacinone); delta-9-tetrahydrocannabinol (dronabinol, MARINOL®); beta-lapachone; lapachol; colchicines; betulinic acid; a camptothecin (including the synthetic analogue topotecan (HYCAMTIN®), CPT-11 (irinotecan, CAMPTOSAR®), acetylcamptothecin, scopolectin, and 9-aminocamptothecin); bryostatin; callystatin; CC-1065 (including its adozelesin, carzelesin and bizelesin synthetic analogues); podophyllotoxin; podophyllinic acid; teniposide; cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e. g., calicheamicin, especially calicheamicin gamma1I and calicheamicin omegaI1 (see, e.g., Agnew, Chem Intl. Ed. Engl., 33: 183-186 (1994)); dynemicin, including dynemicin A; an esperamicin; as well as neocarzinostatin chromophore and related chromoprotein enediyne antiobiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine (ELDISINE®, FILDESIN®); dacarbazine; mannomustine; mitobronitol; mitolactol; pipobroman; gacytosine; arabinoside (“Ara-C”); thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhóne-Poulenc Rorer, Antony, France); chloranbucil; gemcitabine (GEMZAR®); 6-thioguanine; mercaptopurine; methotrexate; platinum analogs such as cisplatin and carboplatin; vinblastine (VELBAN®); platinum; etoposide (VP-16); ifosfamide; mitoxantrone; vincristine (ONCOVIN®); oxaliplatin; leucovovin; vinorelbine (NAVELBINE®); novantrone; edatrexate; daunomycin; aminopterin; ibandronate; topoisomerase inhibitor RFS 2000; difluorometlhylornithine (DMFO); retinoids such as retinoic acid; capecitabine (XELODA®); pharmaceutically acceptable salts, acids or derivatives of any of the above; as well as combinations of two or more of the above such as CHOP, an abbreviation for a combined therapy of cyclophosphamide, doxorubicin, vincristine, and prednisolone, and FOLFOX, an abbreviation for a treatment regimen with oxaliplatin (ELOXATIN™) combined with 5-FU and leucovovin.

Also included in this definition of chemotherapeutic agents are anti-hormonal agents that act to regulate, reduce, block, or inhibit the effects of hormones that can promote the growth of cancer, and are often in the form of systemic, or whole-body, treatment. They may be hormones themselves. Examples include anti-estrogens and selective estrogen receptor modulators (SERMs), including, for example, tamoxifen (including NOLVADEX® tamoxifen), EVISTA® raloxifene, droloxifene, 4-hydroxytamoxifen, trioxifene, keoxifene, LY117018, onapristone, and FARESTON® toremifene; anti-progesterones; estrogen receptor down-regulators (ERDs); agents that function to suppress or shut down the ovaries, for example, leutinizing hormone-releasing hormone (LHRH) agonists such as LUPRON® and ELIGARD® leuprolide acetate, goserelin acetate, buserelin acetate and tripterelin; other anti-androgens such as flutamide, nilutamide and bicalutamide; and aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® letrozole, and ARIMIDEX® anastrozole. In addition, such definition of chemotherapeutic agents includes bisphosphonates such as clodronate (for example, BONEFOS® or OSTAC®), DIDROCAL® etidronate, NE-58095, ZOMETA® zoledronic acid/zoledronate, FOSAMAX® alendronate, AREDIA® pamidronate, SKELID® tiludronate, or ACTONEL® risedronate; as well as troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as THERATOPE® vaccine and gene therapy vaccines, for example, ALLOVECTIN® vaccine, LEUVECTIN® vaccine, and VAXID® vaccine; LURTOTECAN® topoisomerase 1 inhibitor; ABARELIX® rmRH; lapatinib ditosylate (an ErbB-2 and EGFR dual tyrosine kinase small-molecule inhibitor also known as GW572016); and pharmaceutically acceptable salts, acids or derivatives of any of the above.

Another group of compounds that may be selected as neurological drugs for cancer treatment or prevention are anti-cancer immunoglobulins (including, but not limited to, trastuzumab, pertuzumab, bevacizumab, alemtuxumab, cetuximab, gemtuzumab ozogamicin, ibritumomab tiuxetan, panitumumab and rituximab). In some instances, antibodies in conjunction with a toxic label or conjugate may be used to target and kill desired cells (e.g., cancer cells), including, but not limited to, tositumomab with a ¹³¹I radiolabel, or trastuzumab emtansine.

For example, and without limitation, ocular diseases and disorders may be treated according to the therapeutic methods and with the compositions disclosed herein. Ocular diseases or disorders are diseases or disorders of the eye, which for the purposes herein is considered a CNS organ segregated by the BBB. Ocular diseases or disorders include, but are not limited to, disorders of sclera, cornea, iris and ciliary body (e.g., scleritis, keratitis, corneal ulcer, corneal abrasion, snow blindness, arc eye, Thygeson's superficial punctate keratopathy, corneal neovascularisation, Fuchs' dystrophy, keratoconus, keratoconjunctivitis sicca, iritis and uveitis), disorders of the lens (e.g., cataract), disorders of choroid and retina (e.g., retinal detachment, retinoschisis, hypertensive retinopathy, diabetic retinopathy, retinopathy, retinopathy of prematurity, age-related macular degeneration, macular degeneration (wet or dry), epiretinal membrane, retinitis pigmentosa and macular edema), glaucoma, floaters, disorders of optic nerve and visual pathways (e.g., Leber's hereditary optic neuropathy and optic disc drusen), disorders of ocular muscles/binocular movement accommodation/refraction (e.g., strabismus, ophthalmoparesis, progressive external opthalmoplegia, esotropia, exotropia, hypermetropia, myopia, astigmatism, anisometropia, presbyopia and ophthalmoplegia), visual disturbances and blindness (e.g., amblyopia, Lever's congenital amaurosis, scotoma, color blindness, achromatopsia, nyctalopia, blindness, river blindness and micro-opthalmia/coloboma), red eye, Argyll Robertson pupil, keratomycosis, xerophthalmia and andaniridia.

For an ocular disease or disorder, a neurological drug may be selected that is an anti-angiogenic ophthalmic agent (e.g., bevacizumab, ranibizumab and pegaptanib), an ophthalmic glaucoma agent (e.g., carbachol, epinephrine, demecarium bromide, apraclonidine, brimonidine, brinzolamide, levobunolol, timolol, betaxolol, dorzolamide, bimatoprost, carteolol, metipranolol, dipivefrin, travoprost and latanoprost), a carbonic anhydrase inhibitor (e.g., methazolamide and acetazolamide), an ophthalmic antihistamine (e.g., naphazoline, phenylephrine and tetrahydrozoline), an ocular lubricant, an ophthalmic steroid (e.g., fluorometholone, prednisolone, loteprednol, dexamethasone, difluprednate, rimexolone, fluocinolone, medrysone and triamcinolone), an ophthalmic anesthetic (e.g., lidocaine, proparacaine and tetracaine), an ophthalmic anti-infective (e.g., levofloxacin, gatifloxacin, ciprofloxacin, moxifloxacin, chloramphenicol, bacitracin/polymyxin b, sulfacetamide, tobramycin, azithromycin, besifloxacin, norfloxacin, sulfisoxazole, gentamicin, idoxuridine, erythromycin, natamycin, gramicidin, neomycin, ofloxacin, trifluridine, ganciclovir, vidarabine), an ophthalmic anti-inflammatory agent (e.g., nepafenac, ketorolac, flurbiprofen, suprofen, cyclosporine, triamcinolone, diclofenac and bromfenac), and an ophthalmic antihistamine or decongestant (e.g., ketotifen, olopatadine, epinastine, naphazoline, cromolyn, tetrahydrozoline, pemirolast, bepotastine, naphazoline, phenylephrine, nedocromil, lodoxamide, phenylephrine, emedastine and azelastine).

Viral or microbial infections of the CNS include, but are not limited to, infections by viruses (e.g., influenza, HIV, poliovirus, rubella), bacteria (e.g., Neisseria sp., Streptococcus sp., Pseudomonas sp., Proteus sp., E. coli, S. aureus, Pneumococcus sp., Meningococcus sp., Haemophilus sp., and Mycobacterium tuberculosis) and other microorganisms such as fungi (e.g., yeast, Cryptococcus neoformans), parasites (e.g., toxoplasma gondii) or amoebas resulting in CNS pathophysiologies including, but not limited to, meningitis, encephalitis, myelitis, vasculitis and abscess, which can be acute or chronic.

For a viral or microbial disease, a neurological drug may be selected that includes, but is not limited to, an antiviral compound (including, but not limited to, an adamantane antiviral (e.g., rimantadine and amantadine), an antiviral interferon (e.g., peginterferon alfa-2b), a chemokine receptor antagonist (e.g., maraviroc), an integrase strand transfer inhibitor (e.g., raltegravir), a neuraminidase inhibitor (e.g., oseltamivir and zanamivir), a non-nucleoside reverse transcriptase inhibitor (e.g., efavirenz, etravirine, delavirdine and nevirapine), a nucleoside reverse transcriptase inhibitors (tenofovir, abacavir, lamivudine, zidovudine, stavudine, entecavir, emtricitabine, adefovir, zalcitabine, telbivudine and didanosine), a protease inhibitor (e.g., darunavir, atazanavir, fosamprenavir, tipranavir, ritonavir, nelfinavir, amprenavir, indinavir and saquinavir), a purine nucleoside (e.g., valacyclovir, famciclovir, acyclovir, ribavirin, ganciclovir, valganciclovir and cidofovir), and a miscellaneous antiviral (e.g., enfuvirtide, foscarnet, palivizumab and fomivirsen)), an antibiotic (including, but not limited to, an aminopenicillin (e.g., amoxicillin, ampicillin, oxacillin, nafcillin, cloxacillin, dicloxacillin, flucoxacillin, temocillin, azlocillin, carbenicillin, ticarcillin, mezlocillin, piperacillin and bacampicillin), a cephalosporin (e.g., cefazolin, cephalexin, cephalothin, cefamandole, ceftriaxone, cefotaxime, cefpodoxime, ceftazidime, cefadroxil, cephradine, loracarbef, cefotetan, cefuroxime, cefprozil, cefaclor, and cefoxitin), a carbapenem/penem (e.g., imipenem, meropenem, ertapenem, faropenem and doripenem), a monobactam (e.g., aztreonam, tigemonam, norcardicin A and tabtoxinine-beta-lactam, a beta-lactamase inhibitor (e.g., clavulanic acid, tazobactam and sulbactam) in conjunction with another beta-lactam antibiotic, an aminoglycoside (e.g., amikacin, gentamicin, kanamycin, neomycin, netilmicin, streptomycin, tobramycin, and paromomycin), an ansamycin (e.g., geldanamycin and herbimycin), a carbacephem (e.g., loracarbef), a glycopeptides (e.g., teicoplanin and vancomycin), a macrolide (e.g., azithromycin, clarithromycin, dirithromycin, erythromycin, roxithromycin, troleandomycin, telithromycin and spectinomycin), a monobactam (e.g., aztreonam), a quinolone (e.g., ciprofloxacin, enoxacin, gatifloxacin, levofloxacin, lomefloxacin, moxifloxacin, norfloxacin, ofloxacin, trovafloxacin, grepafloxacin, sparfloxacin and temafloxacin), a sulfonamide (e.g., mafenide, sulfonamidochrysoidine, sulfacetamide, sulfadiazine, sulfamethizole, sulfanilamide, sulfasalazine, sulfisoxazole, trimethoprim, trimethoprim and sulfamethoxazole), a tetracycline (e.g., tetracycline, demeclocycline, doxycycline, minocycline and oxytetracycline), an antineoplastic or cytotoxic antibiotic (e.g., doxorubicin, mitoxantrone, bleomycin, daunorubicin, dactinomycin, epirubicin, idarubicin, plicamycin, mitomycin, pentostatin and valrubicin) and a miscellaneous antibacterial compound (e.g., bacitracin, colistin and polymyxin B)), an antifungal (e.g., metronidazole, nitazoxanide, tinidazole, chloroquine, iodoquinol and paromomycin), and an antiparasitic (including, but not limited to, quinine, chloroquine, amodiaquine, pyrimethamine, sulphadoxine, proguanil, mefloquine, atovaquone, primaquine, artemesinin, halofantrine, doxycycline, clindamycin, mebendazole, pyrantel pamoate, thiabendazole, diethylcarbamazine, ivermectin, rifampin, amphotericin B, melarsoprol, efornithine and albendazole).

CNS inflammation may also be treated according to the methods disclosed herein. Inflammation of the CNS includes, but is not limited to, inflammation that is caused by an injury to the CNS, which can be a physical injury (e.g., due to accident, surgery, brain trauma, spinal cord injury, concussion) and an injury due to or related to one or more other diseases or disorders of the CNS (e.g., abscess, cancer, viral or microbial infection).

For CNS inflammation, a neurological drug may be selected that addresses the inflammation itself (e.g., a nonsteroidal anti-inflammatory agent such as ibuprofen or naproxen), or one which treats the underlying cause of the inflammation (e.g., an anti-viral or anti-cancer agent).

Ischemia of the CNS, as used herein, refers to a group of disorders relating to aberrant blood flow or vascular behavior in the brain or the causes therefor, and includes, but is not limited to: focal brain ischemia, global brain ischemia, stroke (e.g., subarachnoid hemorrhage and intracerebral hemorrhage), and aneurysm.

For ischemia, a neurological drug may be selected that includes, but is not limited to, a thrombolytic (e.g., urokinase, alteplase, reteplase and tenecteplase), a platelet aggregation inhibitor (e.g., aspirin, cilostazol, clopidogrel, prasugrel and dipyridamole), a statin (e.g., lovastatin, pravastatin, fluvastatin, rosuvastatin, atorvastatin, simvastatin, cerivastatin and pitavastatin), and a compound to improve blood flow or vascular flexibility, including, e.g., blood pressure medications.

Neurodegenerative diseases are a group of diseases and disorders associated with neural cell loss of function or death in the CNS, and include, but are not limited to: adrenoleukodystrophy, Alexander's disease, Alper's disease, amyotrophic lateral sclerosis, ataxia telangiectasia, Batten disease, cockayne syndrome, corticobasal degeneration, degeneration caused by or associated with an amyloidosis, Friedreich's ataxia, frontotemporal lobar degeneration, Kennedy's disease, multiple system atrophy, multiple sclerosis, primary lateral sclerosis, progressive supranuclear palsy, spinal muscular atrophy, transverse myelitis, Refsum's disease, and spinocerebellar ataxia.

For a neurodegenerative disease, a neurological drug may be selected that is a growth hormone or neurotrophic factor; examples include but are not limited to brain-derived neurotrophic factor (BDNF), nerve growth factor (NGF), neurotrophin-4/5, fibroblast growth factor (FGF)-2 and other FGFs, neurotrophin (NT)-3, erythropoietin (EPO), hepatocyte growth factor (HGF), epidermal growth factor (EGF), transforming growth factor (TGF)-alpha, TGF-beta, vascular endothelial growth factor (VEGF), interleukin-1 receptor antagonist (IL-1ra), ciliary neurotrophic factor (CNTF), glial-derived neurotrophic factor (GDNF), neurturin, platelet-derived growth factor (PDGF), heregulin, neuregulin, artemin, persephin, interleukins, glial cell line derived neurotrophic factor (GFR), granulocyte-colony stimulating factor (CSF), granulocyte-macrophage-CSF, netrins, cardiotrophin-1, hedgehogs, leukemia inhibitory factor (LIF), midkine, pleiotrophin, bone morphogenetic proteins (BMPs), netrins, saposins, semaphorins, and stem cell factor (SCF).

Seizure diseases and disorders of the CNS involve inappropriate and/or abnormal electrical conduction in the CNS, and include, but are not limited to epilepsy (e.g., absence seizures, atonic seizures, benign Rolandic epilepsy, childhood absence, clonic seizures, complex partial seizures, frontal lobe epilepsy, febrile seizures, infantile spasms, juvenile myoclonic epilepsy, juvenile absence epilepsy, Lennox-Gastaut syndrome, Landau-Kleffner Syndrome, Dravet's syndrome, Otahara syndrome, West syndrome, myoclonic seizures, mitochondrial disorders, progressive myoclonic epilepsies, psychogenic seizures, reflex epilepsy, Rasmussen's Syndrome, simple partial seizures, secondarily generalized seizures, temporal lobe epilepsy, toniclonic seizures, tonic seizures, psychomotor seizures, limbic epilepsy, partial-onset seizures, generalized-onset seizures, status epilepticus, abdominal epilepsy, akinetic seizures, autonomic seizures, massive bilateral myoclonus, catamenial epilepsy, drop seizures, emotional seizures, focal seizures, gelastic seizures, Jacksonian March, Lafora Disease, motor seizures, multifocal seizures, nocturnal seizures, photosensitive seizure, pseudo seizures, sensory seizures, subtle seizures, sylvan seizures, withdrawal seizures, and visual reflex seizures).

For a seizure disorder, a neurological drug may be selected that is an anticonvulsant or antiepileptic including, but not limited to, barbiturate anticonvulsants (e.g., primidone, metharbital, mephobarbital, allobarbital, amobarbital, aprobarbital, alphenal, barbital, brallobarbital and phenobarbital), benzodiazepine anticonvulsants (e.g., diazepam, clonazepam, and lorazepam), carbamate anticonvulsants (e.g. felbamate), carbonic anhydrase inhibitor anticonvulsants (e.g., acetazolamide, topiramate and zonisamide), dibenzazepine anticonvulsants (e.g., rufinamide, carbamazepine, and oxcarbazepine), fatty acid derivative anticonvulsants (e.g., divalproex and valproic acid), gamma-aminobutyric acid analogs (e.g., pregabalin, gabapentin and vigabatrin), gamma-aminobutyric acid reuptake inhibitors (e.g., tiagabine), gamma-aminobutyric acid transaminase inhibitors (e.g., vigabatrin), hydantoin anticonvulsants (e.g. phenytoin, ethotoin, fosphenytoin and mephenytoin), miscellaneous anticonvulsants (e.g., lacosamide and magnesium sulfate), progestins (e.g., progesterone), oxazolidinedione anticonvulsants (e.g., paramethadione and trimethadione), pyrrolidine anticonvulsants (e.g., levetiracetam), succinimide anticonvulsants (e.g., ethosuximide and methsuximide), triazine anticonvulsants (e.g., lamotrigine), and urea anticonvulsants (e.g., phenacemide and pheneturide).

Behavioral disorders are disorders of the CNS characterized by aberrant behavior on the part of the afflicted subject and include, but are not limited to: sleep disorders (e.g., insomnia, parasomnias, night terrors, circadian rhythm sleep disorders, and narcolepsy), mood disorders (e.g., depression, suicidal depression, anxiety, chronic affective disorders, phobias, panic attacks, obsessive-compulsive disorder, attention deficit hyperactivity disorder (ADHD), attention deficit disorder (ADD), chronic fatigue syndrome, agoraphobia, post-traumatic stress disorder, bipolar disorder), eating disorders (e.g., anorexia or bulimia), psychoses, developmental behavioral disorders (e.g., autism, Rett's syndrome, Aspberger's syndrome), personality disorders and psychotic disorders (e.g., schizophrenia, delusional disorder, and the like).

For a behavioral disorder, a neurological drug may be selected from a behavior-modifying compound including, but not limited to, an atypical antipsychotic (e.g., risperidone, olanzapine, apripiprazole, quetiapine, paliperidone, asenapine, clozapine, iloperidone and ziprasidone), a phenothiazine antipsychotic (e.g., prochlorperazine, chlorpromazine, fluphenazine, perphenazine, trifluoperazine, thioridazine and mesoridazine), a thioxanthene (e.g., thiothixene), a miscellaneous antipsychotic (e.g., pimozide, lithium, molindone, haloperidol and loxapine), a selective serotonin reuptake inhibitor (e.g., citalopram, escitalopram, paroxetine, fluoxetine and sertraline), a serotonin-norepinephrine reuptake inhibitor (e.g., duloxetine, venlafaxine, desvenlafaxine, a tricyclic antidepressant (e.g., doxepin, clomipramine, amoxapine, nortriptyline, amitriptyline, trimipramine, imipramine, protriptyline and desipramine), a tetracyclic antidepressant (e.g., mirtazapine and maprotiline), a phenylpiperazine antidepressant (e.g., trazodone and nefazodone), a monoamine oxidase inhibitor (e.g., isocarboxazid, phenelzine, selegiline and tranylcypromine), a benzodiazepine (e.g., alprazolam, estazolam, flurazeptam, clonazepam, lorazepam and diazepam), a norepinephrine-dopamine reuptake inhibitor (e.g., bupropion), a CNS stimulant (e.g., phentermine, diethylpropion, methamphetamine, dextroamphetamine, amphetamine, methylphenidate, dexmethylphenidate, lisdexamfetamine, modafinil, pemoline, phendimetrazine, benzphetamine, phendimetrazine, armodafinil, diethylpropion, caffeine, atomoxetine, doxapram, and mazindol), an anxiolytic/sedative/hypnotic (including, but not limited to, a barbiturate (e.g., secobarbital, phenobarbital and mephobarbital), a benzodiazepine (as described above), and a miscellaneous anxiolytic/sedative/hypnotic (e.g. diphenhydramine, sodium oxybate, zaleplon, hydroxyzine, chloral hydrate, aolpidem, buspirone, doxepin, eszopiclone, ramelteon, meprobamate and ethclorvynol)), a secretin (see, e.g., Ratliff-Schaub et al. Autism 9: 256-265 (2005)), an opioid peptide (see, e.g., Cowen et al., J. Neurochem. 89:273-285 (2004)), and a neuropeptide (see, e.g., Hethwa et al. Am. J. Physiol. 289: E301-305 (2005)).

Lysosomal storage disorders are metabolic disorders which are in some cases associated with the CNS or have CNS-specific symptoms; such disorders include, but are not limited to: Tay-Sachs disease, Gaucher's disease, Fabry disease, mucopolysaccharidosis (types I, II, III, IV, V, VI and VII), glycogen storage disease, GM1-gangliosidosis, metachromatic leukodystrophy, Farber's disease, Canavan's leukodystrophy, and neuronal ceroid lipofuscinoses types 1 and 2, Niemann-Pick disease, Pompe disease, and Krabbe's disease.

For a lysosomal storage disease, a neurological drug may be selected that is itself or otherwise mimics the activity of the enzyme that is impaired in the disease. Exemplary recombinant enzymes for the treatment of lysosomal storage disorders include, but are not limited to those set forth in e.g., U.S. Patent Application publication no. 2005/0142141 (e.g., alpha-L-iduronidase, iduronate-2-sulphatase, N-sulfatase, alpha-N-acetylglucosaminidase, N-acetyl-galactosamine-6-sulfatase, beta-galactosidase, arylsulphatase B, beta-glucuronidase, acid alpha-glucosidase, glucocerebrosidase, alpha-galactosidase A, hexosaminidase A, acid sphingomyelinase, beta-galactocerebrosidase, beta-galactosidase, arylsulfatase A, acid ceramidase, aspartoacylase, palmitoyl-protein thioesterase 1 and tripeptidyl amino peptidase 1).

In one aspect, an antibody of the invention is used to detect a neurological disorder before the onset of symptoms and/or to assess the severity or duration of the disease or disorder. In one aspect, the antibody permits detection and/or imaging of the neurological disorder, including imaging by radiography, tomography, or magnetic resonance imaging (MRI).

Antibodies of the invention can be used either alone or in combination with other agents in a therapy. For instance, an antibody of the invention may be co-administered with at least one additional therapeutic agent. In certain embodiments, an additional therapeutic agent is a chemotherapeutic agent. In another embodiment, the antibody is administered with one or more neurological disorder drugs, as described above.

Such combination therapies noted above encompass combined administration (where two or more therapeutic agents are included in the same or separate formulations), and separate administration, in which case, administration of the antibody of the invention can occur prior to, simultaneously, and/or following, administration of the additional therapeutic agent or agents. In one embodiment, administration of the anti-CD98hc, anti-Bsg, or anti-Glut1 antibody and administration of an additional therapeutic agent occur within about one month, or within about one, two or three weeks, or within about one, two, three, four, five, or six days, of each other. Antibodies of the invention can also be used in combination with radiation therapy.

An antibody of the invention (and any additional therapeutic agent) can be administered, or the methods of the invention can include administration, by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g. by injections, such as intravenous or subcutaneous injections, depending in part on whether the administration is brief or chronic. Various dosing schedules including but not limited to single or multiple administrations over various time-points, bolus administration, and pulse infusion are contemplated herein.

Antibodies of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the formulation, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of an antibody of the invention (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 μg/kg to 15 mg/kg (e.g., 0.1 mg/kg to 10 mg/kg) of antibody can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the antibody). An initial higher loading dose, followed by one or more lower doses may be administered. An exemplary dosing regimen comprises administering e.g., “an initial loading dose of about 4 mg/kg, followed by a weekly maintenance dose of about 2 mg/kg of the antibody. However, other dosage regimens may be useful. The progress of this therapy is easily monitored by conventional techniques and assays.

It is understood that any of the above formulations or therapeutic methods may be carried out using an immunoconjugate of the invention in place of or in addition to an anti-CD98hc, anti-Bsg, or anti-Glut1 antibody.

C. Exemplary Antibodies

1. Exemplary Anti-Basigin Antibodies

In some embodiments, methods provided herein for transporting an agent across the blood-brain barrier can include exposing the blood-brain barrier to an antibody which binds to basigin (Bsg). Methods for generating antibodies, e.g., antibodies that bind to basigin, are known in the art, and described in detail herein. Accordingly, in one aspect, the invention provides isolated antibodies that bind to Bsg. In certain embodiments, an anti-Bsg antibody that binds to a region in the extracellular domain of basigin is provided. In certain embodiments, an anti-Bsg that binds to murine Bsg and/or human Bsg is provided.

In certain embodiments, an anti-Bsg antibody is provided wherein binding of the antibody to basigin does not impair binding of basigin to one or more of its native ligands, e.g., integrin alpha3, integrin alpha6, integrin beta1, cyclophilin A, cyclophilin B, annexin II, and caveolin 1, and/or does not impair transport of any of the native ligands of the BBB-R across the blood-brain barrier. As used herein, “does not impair” means that the one or more native ligands bind and/or is/are transported across the BBB in the same manner (e.g., amount, rate, etc.) as if no antibody were present (i.e., no change in any binding properties).

In certain embodiments, an anti-Bsg antibody is provided wherein binding of Bsg to one or more of its native ligands in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of binding in the absence of the antibody.

In certain embodiments, an anti-Bsg antibody is provided wherein transport of any of the native ligands of Bsg across the blood-brain barrier in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of transport in the absence of the antibody.

In certain embodiments, an anti-Bsg antibody comprises a light chain CDR1 amino acid sequence selected from SEQ ID NOs: 3, 19, 35, 51, and 67, a light chain CDR2 amino acid sequence selected from SEQ ID NOs: 4, 20, 36, 52, and 68, and a light chain CDR3 amino acid sequence selected from SEQ ID NOs: 5, 21, 37, 53, and 69.

In certain embodiments, an anti-Bsg antibody comprises a heavy chain CDR1 amino acid sequence selected from SEQ ID NOs: 6, 22, 38, 54, and 70, a heavy chain CDR2 amino acid sequence selected from SEQ ID NOs: 7, 23, 39, 55, and 71, and a heavy chain CDR3 amino acid sequence selected from SEQ ID NOs: 8, 24, 40, 56, and 72.

In certain embodiments, an anti-Bsg antibody comprises a light chain CDR1 amino acid sequence comprising SEQ ID NO: 3, a light chain CDR2 amino acid sequence comprising SEQ ID NO:4, a light chain CDR3 amino acid sequence comprising SEQ ID NO:5, and a heavy chain CDR1 amino acid sequence comprising SEQ ID NO:6, a heavy chain CDR2 amino acid sequence comprising SEQ ID NO: 7, and a heavy chain CDR3 amino acid sequence comprising SEQ ID NO: 8.

In certain embodiments, an anti-Bsg antibody comprises a light chain CDR1 amino acid sequence comprising SEQ ID NO: 19, a light chain CDR2 amino acid sequence comprising SEQ ID NO:20, a light chain CDR3 amino acid sequence comprising SEQ ID NO:21, and a heavy chain CDR1 amino acid sequence comprising SEQ ID NO:22, a heavy chain CDR2 amino acid sequence comprising SEQ ID NO: 23, and a heavy chain CDR3 amino acid sequence comprising SEQ ID NO:24.

In certain embodiments, an anti-Bsg antibody comprises a light chain CDR1 amino acid sequence comprising SEQ ID NO: 35, a light chain CDR2 amino acid sequence comprising SEQ ID NO:36, a light chain CDR3 amino acid sequence comprising SEQ ID NO:37, and a heavy chain CDR1 amino acid sequence comprising SEQ ID NO:38, a heavy chain CDR2 amino acid sequence comprising SEQ ID NO: 39, and a heavy chain CDR3 amino acid sequence comprising SEQ ID NO:40.

In certain embodiments, an anti-Bsg antibody comprises a light chain CDR1 amino acid sequence comprising SEQ ID NO: 51, a light chain CDR2 amino acid sequence comprising SEQ ID NO: 52, and a light chain CDR3 amino acid sequence comprising SEQ ID NO: 53, and a heavy chain CDR1 amino acid sequence comprising SEQ ID NO: 54, a heavy chain CDR2 amino acid sequence comprising SEQ ID NO: 55, and a heavy chain CDR3 amino acid sequence comprising SEQ ID NO: 56.

In certain embodiments, an anti-Bsg antibody comprises a light chain CDR1 amino acid sequence selected from SEQ ID NOs: 67, a light chain CDR2 amino acid sequence comprising SEQ ID NO: 68, and a light chain CDR3 amino acid sequence comprising SEQ ID NO: 69, and a heavy chain CDR1 amino acid sequence comprising SEQ ID NO: 70, a heavy chain CDR2 amino acid sequence comprising SEQ ID NO: 71, and a heavy chain CDR3 amino acid comprising SEQ ID NO: 72.

In certain embodiments, an anti-Bsg antibody further comprises light chain variable domain framework regions comprising an amino acid sequence selected from SEQ ID NOs: 9, 25, 41, 57, and 73 for FR1, an amino acid sequence selected from SEQ ID NOs: 10. 26, 42, 58, and 74 for FR2, an amino acid sequence selected from SEQ ID NOs: 11, 27, 43, 59, and 75 for FR3, and an amino acid sequence selected from SEQ ID NOs: 12, 28, 44, 60, and 76 for FR4.

In certain embodiments, an anti-Bsg antibody further comprises heavy chain variable domain framework regions comprising an amino acid sequence selected from SEQ ID NOs: 13, 29, 45, 61, and 77 for FR1, an amino acid sequence selected from SEQ ID NOs: 14. 30, 46, 62, and 78 for FR2, an amino acid sequence selected from SEQ ID NOs: 15, 31, 47, 63, and 79 for FR3, and an amino acid sequence selected from SEQ ID NOs:16, 32, 48, 64, and 80 for FR4.

In certain embodiments, an anti-Bsg antibody comprises a light chain comprising a variable domain comprising an amino acid sequence selected from SEQ ID NOs: 1, 17, 33, 49, and 65. In some embodiments, an anti-Bsg antibody comprises a light chain variable domain comprising an amino acid sequence that is at least 85%, at least 90%, at least 91%, at least 92%, at least 93%, at least 94%, at least 95%, at least 96%, at least 97%, at least 98%, or at least 99% identical to an amino acid sequence selected from SEQ ID NOs: 1, 17, 33, 49, and 65.

In certain embodiments, an anti-Bsg antibody comprises a heavy chain comprising a variable domain comprising an amino acid sequence selected from SEQ ID NOs:2, 18, 34, 50, 66, and 66. In some embodiments, an anti-Bsg antibody comprises a heavy chain variable domain comprising an amino acid sequence that is at least 85%, at least 90%, at least 91%, at least 92%, at least 93%, at least 94%, at least 95%, at least 96%, at least 97%, at least 98%, or at least 99% identical to an amino acid sequence selected from SEQ ID NOs:2, 18, 34, 50, and 66.

In certain embodiments, an anti-Bsg antibody comprises a light chain variable domain comprising an amino acid sequence selected from SEQ ID NOs: 1, 17, 33, 49, and 65 and a heavy chain variable domain comprising an amino acid sequence selected from SEQ ID NOs: 2, 18, 34, 50, and 66.

In some embodiments, an anti-Bsg antibody comprises a light chain comprising an amino acid sequence corresponding to SEQ ID NO: 1 and a heavy chain comprising an amino acid sequence corresponding to SEQ ID NO: 2. In one embodiment, the anti-Bsg antibody is anti-Bsg^{A}.

In some embodiments, an anti-Bsg antibody comprises a light chain comprising an amino acid sequence corresponding to SEQ ID NO: 17 and a heavy chain comprising an amino acid sequence corresponding to SEQ ID NO: 18. In one embodiment, the anti-Bsg antibody is anti-Bsg^{B}.

In some embodiments, an anti-Bsg antibody comprises a light chain comprising an amino acid sequence corresponding to SEQ ID NO: 33 and a heavy chain comprising an amino acid sequence corresponding to SEQ ID NO: 34. In one embodiment, the anti-Bsg antibody is anti-Bsg^{C}.

In some embodiments, an anti-Bsg antibody comprises a light chain comprising an amino acid sequence corresponding to SEQ ID NO: 49 and a heavy chain comprising an amino acid sequence corresponding to SEQ ID NO: 50. In one embodiment, the anti-Bsg antibody is anti-Bsg^{D}.

In some embodiments, an anti-Bsg antibody comprises a light chain comprising an amino acid sequence corresponding to SEQ ID NO: 65 and a heavy chain comprising an amino acid sequence corresponding to SEQ ID NO: 66. In one embodiment, the anti-Bsg antibody is anti-Bsg^{E}.

2. Exemplary Anti-Glut Antibodies

In some embodiments, methods provided herein for transporting an agent across the blood-brain barrier can include exposing the blood-brain barrier to an antibody which binds to Glut1. Methods for generating antibodies, e.g., antibodies that bind to Glut1, are known in the art, and described in detail herein (see, e.g., Section C, below). In one aspect, the invention provides isolated antibodies that bind to Glut1. In certain embodiments, an anti-Glut1 that binds to human Glut1 is provided. In certain embodiments, an anti-Glut1 antibody is provided, that binds to murine Glut1 (mGlut1). In certain embodiments, an anti-Glut1 antibody is provided that binds to human Glut1 (hGlut1). In certain embodiments, an anti-Glut1 antibody is provided that binds to hGlut1 and mGlut1.

In certain embodiments, an anti-Glut1 antibody is provided wherein binding of the antibody to Glut1 does not impair binding of Glut1 to one or more of its native ligands and/or does not impair transport of any of the native ligands of the BBB-R across the blood-brain barrier. As used herein, “does not impair” means that the one or more native ligands bind and/or is/are transported across the BBB in the same manner (e.g., amount, rate, etc.) as if no antibody were present (i.e., no change in any binding properties).

In certain embodiments, an anti-Glut1 antibody is provided wherein binding of Glut1 to one or more of its native ligands in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of binding in the absence of the antibody.

In certain embodiments, an anti-Glut1 antibody is provided wherein transport of any of the native ligands of the BBB-R across the blood-brain barrier in the presence of the antibody is at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the amount of transport in the absence of the antibody.

In certain embodiments, an anti-Glut1 antibody comprises a light chain CDR1 amino acid sequence comprising SEQ ID NO: 83, a light chain CDR2 amino acid sequence comprising SEQ ID NO: 84, and a light chain CDR3 amino acid sequence comprising SEQ ID NO: 85.

In certain embodiments, an anti-Glut1 antibody comprises a heavy chain CDR1 amino acid sequence comprising SEQ ID NO: 86, a heavy chain CDR2 amino acid sequence comprising SEQ ID NO: 87, and a heavy chain CDR3 amino acid sequence comprising SEQ ID NO: 88.

In certain embodiments, an anti-Glut1 antibody comprises a light chain CDR1 amino acid sequence comprising SEQ ID NO: 83, a light chain CDR2 amino acid sequence comprising SEQ ID NO: 84, a light chain CDR3 amino acid sequence comprising SEQ ID NO: 85, and a heavy chain CDR1 amino acid sequence comprising SEQ ID NO: 86, a heavy chain CDR2 amino acid sequence comprising SEQ ID NO: 87, and a heavy chain CDR3 amino acid sequence comprising SEQ ID NO: 88.

In certain embodiments, an anti-Glut1 antibody comprises a light chain variable domain comprising framework regions comprising an amino acid sequence corresponding to SEQ ID NO: 89 for FR1, SEQ ID NO: 90 for FR2, SEQ ID NO:91 for FR3, and SEQ ID NO:92 for FR4.

In certain embodiments, an anti-Glut1 antibody comprises a heavy chain variable domain comprising framework regions comprising an amino acid sequence corresponding to SEQ ID NO:93 for FR1, SEQ ID NO:94 for FR2, SEQ ID NO:95 for FR3, and SEQ ID NO:96 for FR4.

In certain embodiments, an anti-Glut1 antibody comprises a light chain variable domain comprising an amino acid sequence corresponding to SEQ ID NO:81 and/or a heavy chain variable domain comprising an amino acid sequence corresponding to SEQ ID NO:82. In certain embodiments, an anti-Glut1 antibody comprises a light chain variable domain comprising an amino acid sequence that is at least 85%, at least 90%, at least 91%, at least 92%, at least 93%, at least 94%, at least 95%, at least 96%, at least 97%, at least 98%, or at least 99% identical to SEQ ID NO:81. In some embodiments, an anti-Glut1 antibody comprises a heavy chain variable domain comprising an amino acid sequence that is at least 85%, at least 90%, at least 91%, at least 92%, at least 93%, at least 94%, at least 95%, at least 96%, at least 97%, at least 98%, or at least 99% identical to SEQ ID NO:82.

D. Features of the Antibodies

In a further aspect of the invention, an anti-CD98hc, anti-basigin, or an anti-Glut1 antibody according to any of the above embodiments is a monoclonal antibody, including a chimeric, humanized or human antibody. In one embodiment, an anti-CD98hc, anti-basigin, or an anti-Glut1 antibody is an antibody fragment, e.g., a Fv, Fab, Fab′, scFv, diabody, or F(ab′)₂ fragment. In another embodiment, the antibody is a full length antibody, e.g., an intact IgG1 antibody or other antibody class or isotype as defined herein.

In a further aspect, an anti-CD98hc, anti-Bsg or anti-Glut1 antibody according to any of the above embodiments may incorporate any of the features, singly or in combination, as described in Sections 1-7 below:

1. Antibody Affinity

In certain embodiments, an antibody provided herein has a dissociation constant (Kd) of ≦1M, ≦100 nM, ≦10 nM, ≦1 nM, ≦0.1 nM, ≦0.01 nM, or ≦0.001 nM (e.g., 10⁻⁸ M or less, e.g. from 10⁻⁸ M to 10⁻¹³ M, e.g., from 10⁻⁹ M to 10⁻¹³ M).

In one embodiment, Kd is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, Kd is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at ˜10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 l/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 l/minute. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio k_{off}/kₒₙ. See, e.g., Chen et al., J. Mol. Biol. 293:865-881 (1999). If the on-rate exceeds 106 M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, an antibody provided herein is an antibody fragment. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthün, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Human Antibodies

In certain embodiments, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3): 185-91 (2005).

Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

5. Library-Derived Antibodies

Antibodies of the invention may be isolated by screening combinatorial libraries for antibodies with the desired activity or activities. For example, a variety of methods are known in the art for generating phage display libraries and screening such libraries for antibodies possessing the desired binding characteristics. Such methods are reviewed, e.g., in Hoogenboom et al. in Methods in Molecular Biology 178:1-37 (O'Brien et al., ed., Human Press, Totowa, N.J., 2001) and further described, e.g., in the McCafferty et al., Nature 348:552-554; Clackson et al., Nature 352: 624-628 (1991); Marks et al., J. Mol. Biol. 222: 581-597 (1992); Marks and Bradbury, in Methods in Molecular Biology 248:161-175 (Lo, ed., Human Press, Totowa, N.J., 2003); Sidhu et al., J. Mol. Biol. 338(2): 299-310 (2004); Lee et al., J. Mol. Biol. 340(5): 1073-1093 (2004); Fellouse, Proc. Natl. Acad. Sci. USA 101(34): 12467-12472 (2004); and Lee et al., J. Immunol. Methods 284(1-2): 119-132(2004).

In certain phage display methods, repertoires of VH and VL genes are separately cloned by polymerase chain reaction (PCR) and recombined randomly in phage libraries, which can then be screened for antigen-binding phage as described in Winter et al., Ann. Rev. Immunol., 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al., EMBO J, 12: 725-734 (1993). Finally, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter, J. Mol. Biol., 227: 381-388 (1992). Patent publications describing human antibody phage libraries include, for example: U.S. Pat. No. 5,750,373, and US Patent Publication Nos. 2005/0079574, 2005/0119455, 2005/0266000, 2007/0117126, 2007/0160598, 2007/0237764, 2007/0292936, and 2009/0002360.

Antibodies or antibody fragments isolated from human antibody libraries are considered human antibodies or human antibody fragments herein.

6. Multispecific Antibodies

In certain embodiments, an antibody provided herein is a multispecific antibody, e.g. a bispecific antibody. Multispecific antibodies are monoclonal antibodies that have binding specificities for at least two different sites. In certain embodiments, one of the binding specificities is for CD98hc and the other is for any other antigen. In certain embodiments, one of the binding specificities is for basigin and the other is for any other antigen. In certain embodiments, one of the binding specificities is for Glut1 and the other is for any other antigen. Bispecific antibodies can be prepared as full length antibodies or antibody fragments.

In some embodiments, an antigen-binding domain of a multispecific antibody (such as a bispecific antibody) comprises two VH/VL units, wherein a first VH/VL unit specifically binds to a first epitope and a second VH/VL unit specifically binds to a second epitope, wherein each VH/VL unit comprises a heavy chain variable domain (VH) and a light chain variable domain (VL). Such multispecific antibodies include, but are not limited to, full length antibodies, antibodies having two or more VL and VH domains, antibody fragments such as Fab, Fv, dsFv, scFv, diabodies, bispecific diabodies and triabodies, antibody fragments that have been linked covalently or non-covalently. A VH/VL unit that further comprises at least a portion of a heavy chain variable region and/or at least a portion of a light chain variable region may also be referred to as an “arm” or “hemimer” or “half antibody.” In some embodiments, a hemimer comprises a sufficient portion of a heavy chain variable region to allow intramolecular disulfide bonds to be formed with a second hemimer. In some embodiments, a hemimer comprises a knob mutation or a hole mutation, for example, to allow heterodimerization with a second hemimer or half antibody that comprises a complementary hole mutation or knob mutation. Knob mutations and hole mutations are discussed further, below.

Techniques for making multispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). The term “knob-into-hole” or “KnH” technology as used herein refers to the technology directing the pairing of two polypeptides together in vitro or in vivo by introducing a protuberance (knob) into one polypeptide and a cavity (hole) into the other polypeptide at an interface in which they interact. For example, KnHs have been introduced in the Fc:Fc binding interfaces, CL:CH1 interfaces or VH/VL interfaces of antibodies (see, e.g., US 2011/0287009, US2007/0178552, WO 96/027011, WO 98/050431, and Zhu et al., 1997, Protein Science 6:781-788). In some embodiments, KnHs drive the pairing of two different heavy chains together during the manufacture of multispecific antibodies. For example, multispecific antibodies having KnH in their Fc regions can further comprise single variable domains linked to each Fc region, or further comprise different heavy chain variable domains that pair with similar or different light chain variable domains. KnH technology can be also be used to pair two different receptor extracellular domains together or any other polypeptide sequences that comprises different target recognition sequences (e.g., including affibodies, peptibodies and other Fc fusions).

The term “knob mutation” as used herein refers to a mutation that introduces a protuberance (knob) into a polypeptide at an interface in which the polypeptide interacts with another polypeptide. In some embodiments, the other polypeptide has a hole mutation.

The term “hole mutation” as used herein refers to a mutation that introduces a cavity (hole) into a polypeptide at an interface in which the polypeptide interacts with another polypeptide. In some embodiments, the other polypeptide has a knob mutation.

Multi-specific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” are also included herein (see, e.g. US 2006/0025576A1).

The antibody or fragment herein also includes a “Dual Acting FAb” or “DAF” comprising an antigen binding site that binds to CD98hc, basigin or Glut1, as well as another, different antigen (see, US 2008/0069820, for example).

In certain embodiments, an antibody disclosed herein, e.g., anti-CD98hc, anti-Bsg, or anti-Glut1 antibody is a multispecific antibody comprising a therapeutic arm that is specific for a “CNS antigen” or “brain antigen”. For example, a multispecific antibody disclosed herein has a first arm that is specific for CD98hc, or Bsg, or Glut1, and a second arm that is specific for a brain antigen. Examples of such antigens include, without limitation: BACE1, Abeta, EGFR, HER2, tau, Apo, e.g., ApoE4, alpha-synuclein, CD20, huntingtin, PrP, LRRK2, parkin, presenilin 1, presenilin 2, gamma secretase, DR6, APP, p75NTR, IL6R, TNFR1, IL1β, and caspase 6. In one embodiment, the antigen is BACE1. In another embodiment, the antigen is Abeta.

Thus, in certain embodiments, one of the binding specificities is for CD98hc and the other is for any other antigen. In certain embodiments, one of the binding specificities is for basigin and the other is for any other antigen. In certain embodiments, one of the binding specificities is for Glut1 and the other is for any other antigen. In certain embodiments, bispecific antibodies may bind to two different epitopes of CD98hc. In certain embodiments, bispecific antibodies may bind to two different epitopes of basigin. In certain embodiments, bispecific antibodies may bind to two different epitopes of Glut1. Furthermore, multispecific antibodies may also be used to localize cytotoxic agents to cells which express CD98hc, Glut1 and/or basigin.

Antibodies that are specific for brain antigens, e.g., those exemplified above, are known in the art. By way of example, anti-BACE1 antibodies are known, and exemplary antibody sequences are described, e.g., in International Patent Publication No. WO 2012/075037. In one embodiment, the extent of binding of an anti-BACE1 antibody to an unrelated, non-BACE1 protein is less than about 10% of the binding of the antibody to BACE1 as measured, e.g., by a radioimmunoassay (RIA). In certain embodiments, an antibody that binds to BACE1 has a dissociation constant (Kd) of ≦1 μM, ≦100 nM, ≦10 nM, ≦1 nM, ≦0.1 nM, ≦0.01 nM, or ≦0.001 nM (e.g. 10⁻⁸ M or less, e.g. from 10⁻⁸ M to 10⁻¹³ M, e.g., from 10⁻⁹ M to 10⁻¹³ M). In certain embodiments, an anti-BACE1 antibody binds to an epitope of BACE1 that is conserved among BACE1 from different species and isoforms.

In one embodiment, an antibody is provided that binds to the epitope on BACE1 bound by anti-BACE1 antibody YW 412.8.31. In other embodiments, an antibody is provided that binds to an exosite within BACE1 located in the catalytic domain of BACE1. In one embodiment an antibody is provided that competes with the peptides identified in Kornacker et al., Biochem. 44:11567-11573 (2005), which is incorporated herein by reference in its entirety, (i.e., Peptides 1, 2, 3, 1-11, 1-10, 1-9, 1-8, 1-7, 1-6, 2-12, 3-12, 4-12, 5-12, 6-12, 7-12, 8-12, 9-12, 10-12, 4, 5, 6, 5-10, 5-9, scrambled, Y5A, P6A, Y7A, F8A, 19A, P10A and L11A) for binding to BACE1.

By way of further example, anti-Abeta antibodies which specifically bind to human Abeta are known. A non-limiting example of an anti-Abeta antibody is crenezumab. Other non-limiting examples of anti-Abeta antibodies are solanezumab, bapineuzumab, gantenerumab, aducanumab, ponezumab, and any anti-Abeta antibodies disclosed in the following publications: WO2000162801, WO2002046237, WO2002003911, WO2003016466, WO2003016467, WO2003077858, WO2004029629, WO2004032868, WO2004032868, WO2004108895, WO2005028511, WO2006039470, WO2006036291, WO2006066089, WO2006066171, WO2006066049, WO2006095041, WO2009027105.

7. Antibody Variants

In certain embodiments, amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

a) Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table C under the heading of “preferred substitutions.” More substantial changes are provided in Table C under the heading of “exemplary substitutions,” and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

### Table C: Conservative Amino Acid Substitutions

TABLE C
Conservative Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more HVR residues are mutated and the variant antibodies displayed on phage and screened for a particular biological activity (e.g. binding affinity).

Alterations (e.g., substitutions) may be made in HVRs, e.g., to improve antibody affinity. Such alterations may be made in HVR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more HVRs so long as such alterations do not substantially reduce the ability of the antibody to bind antigen. For example, conservative alterations (e.g., conservative substitutions as provided herein) that do not substantially reduce binding affinity may be made in HVRs. Such alterations may, for example, be outside of antigen contacting residues in the HVRs. In certain embodiments of the variant VH and VL sequences provided above, each HVR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b) Glycosylation Variants

In certain embodiments, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In one embodiment, antibody variants are provided having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (Eu numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lecl3 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

Antibodies variants are further provided with bisected oligosaccharides, e.g., in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c) Fc Region Variants

In certain embodiments, one or more amino acid modifications may be introduced into the Fc region of an antibody provided herein, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the invention contemplates an antibody variant that possesses some but not all effector functions, which make it a desirable candidate for applications in which the half life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006)).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain embodiments, an antibody variant comprises an Fc region with one or more amino acid substitutions which improve ADCC, e.g., substitutions at positions 298, 333, and/or 334 of the Fc region (EU numbering of residues).

In some embodiments, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826). FcRn binding domain mutations M252Y, S254T and T256E (YTE) have been described to increase FcRn binding and thus increase the half-life of antibodies. See U.S. Published Patent Application No. 2003/0190311 and Dall'Acqua et al., J. Biol. Chem. 281:23514-23524 (2006). Additionally, FcRn binding domain mutations N434A and Y436I (AI) have been described to also increase FcRn binding. See Yeung et al., J. Immunol. 182: 7663-7671 (2009). See also Duncan & Winter, Nature 322:738-40 (1988); U.S. Pat. No. 5,648,260; U.S. Pat. No. 5,624,821; and WO 94/29351 concerning other examples of Fc region variants.

d) Cysteine Engineered Antibody Variants

In certain embodiments, it may be desirable to create cysteine engineered antibodies, e.g., “thioMAbs,” in which one or more residues of an antibody are substituted with cysteine residues. In particular embodiments, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, e.g., in U.S. Pat. No. 7,521,541.

e) Antibody Derivatives

In certain embodiments, an antibody provided herein may be further modified to contain additional nonproteinaceous moieties that are known in the art and readily available.

The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In another embodiment, conjugates of an antibody and nonproteinaceous moiety that may be selectively heated by exposure to radiation are provided. In one embodiment, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

E. Recombinant Methods and Compositions

Antibodies may be produced using recombinant methods and compositions, e.g., as described in U.S. Pat. No. 4,816,567. In one embodiment, isolated nucleic acid encoding an anti-CD98hc, anti-Bsg, or anti-Glut1 antibody described herein is provided. Such nucleic acid may encode an amino acid sequence comprising the VL and/or an amino acid sequence comprising the VH of the antibody (e.g., the light and/or heavy chains of the antibody). In a further embodiment, one or more vectors (e.g., expression vectors) comprising such nucleic acid are provided. In a further embodiment, a host cell comprising such nucleic acid is provided. In one such embodiment, a host cell comprises (e.g., has been transformed with): (1) a vector comprising a nucleic acid that encodes an amino acid sequence comprising the VL of the antibody and an amino acid sequence comprising the VH of the antibody, or (2) a first vector comprising a nucleic acid that encodes an amino acid sequence comprising the VL of the antibody and a second vector comprising a nucleic acid that encodes an amino acid sequence comprising the VH of the antibody. In one embodiment, the host cell is eukaryotic, e.g. a Chinese Hamster Ovary (CHO) cell or lymphoid cell (e.g., Y0, NS0, Sp20 cell). In one embodiment, a method of making an anti-CD98hc, anti-Bsg, or anti-Glut1 antibody is provided, wherein the method comprises culturing a host cell comprising a nucleic acid encoding the antibody, as provided above, under conditions suitable for expression of the antibody, and optionally recovering the antibody from the host cell (or host cell culture medium).

For recombinant production of an anti-CD98hc, anti-Bsg, or anti-Glut1 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. See Gerngross, Nat. Biotech. 22:1409-1414 (2004), and Li et al., Nat. Biotech. 24:210-215 (2006).

Suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003).

F. Assays

Anti-CD98hc, anti-Bsg, or anti-Glut1 antibodies provided herein may be identified, screened for, or characterized for their physical/chemical properties and/or biological activities by various assays known in the art.

1. Binding Assays and Other Assays

In one aspect, an antibody of the invention is tested for its antigen binding activity, e.g., by known methods such as ELISA, Western blot, FACS, etc.

In another aspect, competition assays may be used to identify an antibody that competes with an anti-basigin antibody disclosed herein (e.g., anti-Bsg^{A}c anti-Bsg^{B}, anti-Bsg^{C}, anti-Bsg^{D}, and anti-Bsg^{E}) for binding to Bsg. In some embodiments, an antibody according to the present disclosure competes with anti-Bsg^{A}. In some embodiments, an antibody according to the present disclosure competes with anti-Bsg^{B}. In some embodiments, an antibody according to the present disclosure competes with anti-Bsg^{C}. In some embodiments, an antibody according to the present disclosure competes with anti-Bsg^{D}. In some embodiments, an antibody according to the present disclosure competes with anti-Bsg^{C} and anti-Bsg^{D}. In some embodiments, an antibody according to the present disclosure competes with anti-Bsg^{E}.

In certain embodiments, such a competing antibody binds to the same epitope (e.g., a linear or a conformational epitope) that is bound by one of anti-Bsg,^{A} anti-Bsg^{B}, anti-Bsg^{C}, anti-Bsg^{D}, and anti-Bsg^{E}, disclosed herein.

In another aspect, competition assays may be used to identify an antibody that competes with an anti-Glut1 antibody disclosed herein (e.g., the anti-Glut1 antibody having light chain variable region sequence of SEQ ID NO: 81, and heavy chain variable region sequence of SEQ ID NO: 82) for binding to Glut1.

In certain embodiments, such a competing antibody binds to the same epitope (e.g., a linear or a conformational epitope) that is bound by the Glut1 antibody disclosed herein (e.g., the anti-Glut1 antibody having light chain variable region sequence of SEQ ID NO: 81, and heavy chain variable region sequence of SEQ ID NO: 82).

Detailed exemplary methods for mapping an epitope to which an antibody binds are provided in Morris (1996) “Epitope Mapping Protocols,” in Methods in Molecular Biology vol. 66 (Humana Press, Totowa, N.J.).

In an exemplary competition assay, immobilized Bsg is incubated in a solution comprising a first labeled antibody that binds to Bsg (e.g., any of anti-Bsg,^{A} anti-Bsg^{B}, anti-Bsg^{C}, anti-Bsg^{D}, and anti-Bsg^{E}, disclosed herein) and a second unlabeled antibody that is being tested for its ability to compete with the first antibody for binding to Bsg. The second antibody may be present in a hybridoma supernatant. As a control, immobilized Bsg is incubated in a solution comprising the first labeled antibody but not the second unlabeled antibody. After incubation under conditions permissive for binding of the first antibody to Bsg, excess unbound antibody is removed, and the amount of label associated with immobilized Bsg is measured. If the amount of label associated with immobilized Bsg is substantially reduced in the test sample relative to the control sample, then that indicates that the second antibody is competing with the first antibody for binding to Bsg. See Harlow and Lane (1988) Antibodies: A Laboratory Manual ch.14 (Cold Spring Harbor Laboratory, Cold Spring Harbor, N.Y.).

In an exemplary competition assay, immobilized Glut1 is incubated in a solution comprising a first labeled antibody that binds to Glut1 herein (e.g., the anti-Glut1 antibody having light chain variable region sequence of SEQ ID NO: 81, and heavy chain variable region sequence of SEQ ID NO: 82) and a second unlabeled antibody that is being tested for its ability to compete with the first antibody for binding to Glutl. The second antibody may be present in a hybridoma supernatant. As a control, immobilized Glut1 is incubated in a solution comprising the first labeled antibody but not the second unlabeled antibody. After incubation under conditions permissive for binding of the first antibody to Glut1, excess unbound antibody is removed, and the amount of label associated with immobilized Glut1 is measured. If the amount of label associated with immobilized Glut1 is substantially reduced in the test sample relative to the control sample, then that indicates that the second antibody is competing with the first antibody for binding to Glut1. See Harlow and Lane (1988) Antibodies: A Laboratory Manual ch. 14 (Cold Spring Harbor Laboratory, Cold Spring Harbor, N.Y.).

2. Activity Assays

In one aspect, assays are provided for identifying anti-CD98hc antibodies thereof having biological activity. In one aspect, assays are provided for identifying anti-Bsg antibodies thereof having biological activity. In one aspect, assays are provided for identifying anti-Glut1 antibodies thereof having biological activity.

CD98hc Activity Assays

Biological activity may include, e.g., amino acid transport for CD98hc. Antibodies having such biological activity in vivo and/or in vitro are also provided.

In certain embodiments, an antibody disclosed herein may be tested for such biological activity.

In some embodiments, antibodies for use according to the methods disclosed herein (e.g., using anti-CD98hc antibodies) do not inhibit cell proliferation or division. In some embodiments, antibodies for use according to the methods disclosed herein (e.g., anti-CD98hc antibodies) do not inhibit cell adhesion. Assays for measuring the effect of a CD98hc-binding antibody on cell proliferation, cell division, apoptosis and cell adhesion can be performed, by way of example and without limitation, according to the methods described in U.S. Patent Application Publication No. 2013/0052197. See also, Yagita H. et al. (1986) Cancer Res. 46:1478-1484; and Warren A. P., et al. (1996) Blood 87:3676-3687. Any other suitable methods known in the art may also be used to test the activity of CD98hc-binding antibodies.

In some embodiments, the anti-CD98hc antibodies do not inhibit amino acid transport. In vitro assay which may be used to detect amino acid transport by CD98hc (e.g., in a heterodimeric complex with a CD98 light chain (e.g., LAT1, LAT2, y+LAT1, y+LAT2, xCT, and Asc-1) are known and described in the art. See, e.g., Fenczik, C. A et al. (2001) J. Biol. Chem. 276, 8746-8752; see also, US 2013/0052197.

In a specific embodiments, the kinetics of amino acid transport of any of the native ligands of the CD98 heterodimeric complex across the blood-brain barrier in the presence of the anti-CD98hc antibody are at least 10% (e.g., 10%, 15%, 20%, 25%, 30%, 35%, 40%, 50%, 55%, 60%, 65%, 70%, 75%, 80%, 85%, 90%, 91%, 92%, 93%, 94%, 95%, 96%, 97%, 98%, 99%, or 100%) of the transport kinetics in the absence of the antibody, wherein the kinetics in the absence of the antibody are one or more of:

K_{M}=295 μM for glutamine (in the presence of NaCl);

K_{M}=236 μM for leucine (in the presence of NaCl);

K_{M}=120 μM for arginine (in the presence of NaCl); and

K_{M}=138 μM for arginine (in the absence of NaCl).

The amino acid transport kinetics of the CD98 amino acid antiporter can be measured in an assay as described, e.g., by Broer et al. Biochem J. 2000 Aug. 1; 349 Pt 3:787-95.

In one aspect, assays are provided for identifying anti-CD98hc/BACE1 multispecific antibodies having biological activity. In another aspect, assays are provided for identifying anti-Bsg/BACE1 multispecific antibodies having biological activity. In another aspect, assays are provided for identifying anti-Glut1/BACE1 multispecific antibodies having biological activity. Biological activity may include, e.g., inhibition of BACE1 aspartyl protease activity. Antibodies having such biological activity in vivo and/or in vitro are also provided, e.g., as evaluated by homogeneous time-resolved fluorescence HTRF assay or a microfluidic capillary electrophoretic (MCE) assay using synthetic substrate peptides, or in vivo in cell lines which express BACE1 substrates such as APP.

In another aspect, assays are provided for measuring brain uptake of the antibodies disclosed herein (e.g., anti-CD98hc, Bsg, or Glut1 antibodies). Such assays are described, e.g., in the Examples below.

In another aspect, assays are provided for measuring amyloid beta in brain and plasma, including assays for determining increase or reduction in brain amyloid beta, and increase or reduction in plasma amyloid beta. Such assays are described herein, e.g., in the Examples below.

By way of example, an antibody disclosed herein may be conjugated to an imaging agent. Following administration of the antibody conjugate, the imaging agent may be detected, e.g., in isolated brain tissue, and/or using in vivo brain imaging techniques (e.g., using bioluminescence or fluorescence) (see, e.g., J. R. Martin. J Neurogenet. 2008; 22(3):285-307).

3. Affinity Assays

In certain embodiments, the invention provides a method of making an antibody useful for transporting an agent (e.g., a neurological disorder drug or imaging agent) across the blood-brain barrier comprising selecting an antibody from a panel of antibodies against a BBB-R because it has a low affinity for the BBB-R, e.g., an affinity for the BBB-R which is in the range from about 5 nM, or from about 20 nM, or from about 100 nM, to about 10 μM, or to about 1 μM, or to about 500 mM. Thus, the affinity may be in the range from about 5 nM to about 10 μM or in the range from about 20 nM to about 1 μM, or in the range from about 100 nM to about 500 nM, e.g. as measured by Scatchard analysis or BIACORE®. As will be understood by one of ordinary skill in the art, conjugating a heterologous molecule/compound to an antibody can decrease the affinity of the antibody for its target due, e.g., to steric hindrance or even to elimination of one binding arm if the antibody is made multispecific with one or more arms binding to a different antigen than the antibody's original target. In one embodiment, a low affinity antibody of the invention specific for CD98hc, basigin, or Glut1, conjugated to BACE1 has a Kd for CD98hc, basigin, or Glut1, as measured by BIACORE, of about 30 nM. In another embodiment, a low affinity antibody of the invention specific for CD98hc, basigin, or Glut1, conjugated to BACE1 has a Kd for CD98hc, basigin, or Glut1, as measured by BIACORE, of about 600 nM.

One exemplary assay for evaluating antibody affinity is by Scatchard analysis. For example, the anti-BBB-R antibody of interest can be iodinated using the lactoperoxidase method (Bennett and Horuk, Methods in Enzymology 288 pg. 134-148 (1997)). A radiolabeled anti-BBB-R antibody is purified from free ¹²⁵I-Na by gel filtration using a NAP-5 column and its specific activity measured. Competition reaction mixtures of 50 μl containing a fixed concentration of iodinated antibody and decreasing concentrations of serially diluted unlabeled antibody are placed into 96-well plates. Cells transiently expressing BBB-R are cultured in growth media, consisting of Dulbecco's modified eagle's medium (DMEM) (Genentech) supplemented with 10% FBS, 2 mM L-glutamine and 1×penicillin-streptomycin at 37° C. in 5% C02. Cells are detached from the dishes using Sigma Cell Dissociation Solution and washed with binding buffer (DMEM with 1% bovine serum albumin, 50 mM HEPES, pH 7.2, and 0.2% sodium azide). The washed cells are added at an approximate density of 200,000 cells in 0.2 mL of binding buffer to the 96-well plates containing the 50 μl competition reaction mixtures. The final concentration of the unlabeled antibody in the competition reaction with cells is varied, starting at 1000 nM and then decreasing by 1:2 fold dilution for 10 concentrations and including a zero-added, buffer-only sample. Competition reactions with cells for each concentration of unlabeled antibody are assayed in triplicate. Competition reactions with cells are incubated for 2 hours at room temperature. After the 2-hour incubation, the competition reactions are transferred to a filter plate and washed four times with binding buffer to separate free from bound iodinated antibody. The filters are counted by gamma counter and the binding data are evaluated using the fitting algorithm of Munson and Rodbard (1980) to determine the binding affinity of the antibody.

According to another embodiment, Kd is measured using surface plasmon resonance assays with a BIACORE®-2000 device (BIAcore, Inc., Piscataway, N.J.) at 25° C. using anti-human Fc kit (BiAcore Inc., Piscataway, N.J.). Briefly, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Anti-human Fc antibody is diluted with 10 mM sodium acetate, pH 4.0, to 50 μg/ml before injection at a flow rate of 5 μi/minute to achieve approximately 10000 response units (RU) of coupled protein. Following the injection of antibody, 1 M ethanolamine is injected to block unreacted groups. For kinetics measurements, anti-BBB-R antibody variants are injected in HBS-P to reach about 220 RU, then two-fold serial dilutions of BBB-R-His (0.61 nM to 157 nM) are injected in HBS-P at 25° C. at a flow rate of approximately 30 μi/minute. Association rates (kon) and dissociation rates (koff) are calculated using a simple one-to-one Langmuir binding model (BIACORE® Evaluation Software version 3.2) by simultaneously fitting the association and dissociation sensorgrams. The equilibrium dissociation constant (Kd) is calculated as the ratio koff/kon. See, e.g., Chen et al, J. Mol. Biol 293:865-881 (1999).

A surrogate measurement for the affinity of one or more antibodies for the BBB-R is its half maximal inhibitory concentration (IC₅₀), a measure of how much of the antibody is needed to inhibit the binding of a known BBB-R ligand to the BBB-R by 50%. Several methods of determining the IC₅₀ for a given compound are art-known; a common approach is to perform a competition binding assay. In general, a high IC₅₀ indicates that more of the antibody is required to inhibit binding of the known ligand, and thus that the antibody's affinity for that ligand is relatively low. Conversely, a low IC₅₀ indicates that less of the antibody is required to inhibit binding of the known ligand, and thus that the antibody's affinity for that ligand is relatively high.

An exemplary competitive ELISA assay to measure IC₅₀ is one in which increasing concentrations of anti-CD98hc or anti-CD98hc/brain antigen (e.g., anti-CD98hc/BACEl, anti-CD98hc/Abeta, and the like) variant antibodies are used to compete against biotinylated anti-CD98hc antibody for binding to CD98hc. The anti-CD98hc competition ELISA is performed in Maxisorp plates (Neptune, N.J.) coated with 2.5 μg/ml of purified murine CD98hc extracellular domain in PBS at 4° C. overnight. Plates are washed with PBS/0.05%>Tween 20 and blocked using Superblock blocking buffer in PBS (Thermo Scientific, Hudson, N.H.). A titration of each individual anti-CD98hc or anti-CD98hc/brain antigen (e.g., anti-CD98hc/BACEl or anti-CD98hc/Abeta) (1:3 serial dilution) can be combined with biotinylated anti-CD98hc (0.5 nM final concentration) and added to the plate for 1 hour at room temperature. Plates are washed with PBS/0.05% Tween 20, and HRP-streptavidin (Southern Biotech, Birmingham) is added to the plate and incubated for 1 hour at room temperature. Plates were washed with PBS/0.05% Tween 20, and biotinylated anti-CD98hc bound to the plate is detected using TMB substrate (BioFX Laboratories, Owings Mills). The same type of assay can be performed for, e.g., anti-Glut1 and anti-basigin antibodies.

G. Immunoconjugates

The invention also provides immunoconjugates comprising an anti-CD98hc antibody, or an anti-Bsg antibody, or an anti-Glut1 antibody disclosed herein conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes.

In one embodiment, the antibody herein is coupled with a neurological disorder drug, a chemotherapeutic agent and/or an imaging agent in order to more efficiently transport the drug, chemotherapeutic agent and/or the imaging agent across the BBB.

Covalent conjugation can either be direct or via a linker. In certain embodiments, direct conjugation is by construction of a protein fusion (e.g., by genetic fusion of the two genes encoding the antibody and e.g., the neurological disorder drug and expression as a single protein). In certain embodiments, direct conjugation is by formation of a covalent bond between a reactive group on one of the two portions of the antibody and a corresponding group or acceptor on the, e.g., neurological drug. In certain embodiments, direct conjugation is by modification (e.g., genetic modification) of one of the two molecules to be conjugated to include a reactive group (as non-limiting examples, a sulfhydryl group or a carboxyl group) that forms a covalent attachment to the other molecule to be conjugated under appropriate conditions. As one non-limiting example, a molecule (e.g., an amino acid) with a desired reactive group (e.g., a cysteine residue) may be introduced into the antibody and a disulfide bond formed with the e.g., neurological drug. Methods for covalent conjugation of nucleic acids to proteins are also known in the art (e.g., photocrosslinking, see, e.g., Zatsepin et al. Russ. Chem. Rev. 74: 77-95 (2005)).

Non-covalent conjugation can be by any non-covalent attachment means, including hydrophobic bonds, ionic bonds, electrostatic interactions, and the like, as will be readily understood by one of ordinary skill in the art.

Conjugation may also be performed using a variety of linkers. For example, an antibody and a neurological drug may be conjugated using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. Peptide linkers, comprised of from one to twenty amino acids joined by peptide bonds, may also be used. In certain such embodiments, the amino acids are selected from the twenty naturally-occurring amino acids. In certain other such embodiments, one or more of the amino acids are selected from glycine, alanine, proline, asparagine, glutamine and lysine. The linker may be a “cleavable linker” facilitating release of the neurological drug upon delivery to the brain. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The invention herein expressly contemplates, but is not limited to, conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

In one embodiment, an immunoconjugate is an antibody-drug conjugate (ADC) in which an antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In another embodiment, an immunoconjugate comprises an antibody as described herein conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), momordica charantia inhibitor, curcin, crotin, sapaonaria officinalis inhibitor, gelonin, mitogellin, restrictocin, phenomycin, enomycin, and the tricothecenes.

In another embodiment, an immunoconjugate comprises an antibody as described herein conjugated to a radioactive atom to form a radioconjugate. A variety of radioactive isotopes are available for the production of radioconjugates. Examples include At211, I131, 1125, Y90, Re186, Re188, Sm153, Bi212, P32, Pb212 and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

H. Methods and Compositions for Detection

In some aspects, methods of detecting CD98hc on the blood-brain barrier are provided. Thus, in some aspects, an anti-CD98hc antibody binds to CD98hc with sufficient affinity such that the antibody is useful as a detection agent in targeting CD98hc. In certain embodiments, the anti-CD98hc antibody is useful for detecting the presence of CD98hc in a biological sample. In certain aspects, any of the anti-Bsg antibodies provided herein are useful for detecting the presence of Bsg in a biological sample. In certain embodiments, any of the anti-Glut1 antibodies provided herein are useful for detecting the presence of Glut1 in a biological sample. The term “detecting” as used herein encompasses quantitative or qualitative detection. In certain embodiments, a biological sample comprises a cell or tissue, such as brain cells, e.g., brain capillary endothelial cells.

In one embodiment, an anti-CD98hc antibody for use in a method of detection is provided. In a further aspect, a method of detecting the presence of CD98hc in a biological sample is provided. In certain embodiments, the method comprises contacting the biological sample with an anti-CD98hc antibody as described herein under conditions permissive for binding of the anti-CD98hc antibody to CD98hc, and detecting whether a complex is formed between the anti-CD98hc antibody and CD98hc.

In one embodiment, an anti-Bsg antibody for use in a method of detection is provided. In a further aspect, a method of detecting the presence of Bsg in a biological sample is provided. In certain embodiments, the method comprises contacting the biological sample with an anti-Bsg antibody as described herein under conditions permissive for binding of the anti-Bsg antibody to Bsg, and detecting whether a complex is formed between the anti-Bsg antibody and Bsg. Such method may be an in vitro or in vivo method.

In one embodiment, an anti-Glut1 antibody for use in a method of detection is provided. In a further aspect, a method of detecting the presence of Glut1 in a biological sample is provided. In certain embodiments, the method comprises contacting the biological sample with an anti-Glut1 antibody as described herein under conditions permissive for binding of the anti-Glut1 antibody to Glut1, and detecting whether a complex is formed between the anti-Glut1 antibody and Glut1. Such method may be an in vitro or in vivo method.

In one embodiment, the intact antibody lacks effector function. In another embodiment, the intact antibody has reduced effector function. In another embodiment, the intact antibody is engineered to have reduced effector function. In one aspect, the antibody is a Fab. In another aspect, the antibody has one or more Fc mutations reducing or eliminating effector function. In another aspect, the antibody has modified glycosylation due, e.g., to producing the antibody in a system lacking normal human glycosylation enzymes. In another aspect, the Ig backbone is modified to one which naturally possesses limited or no effector function.

Various techniques are available for determining binding of the antibody to CD98hc, Bsg, and/or Glut1. One such assay is an enzyme linked immunosorbent assay (ELISA) for confirming an ability to bind to human CD98hc, Bsg, and/or Glut1. According to this assay, plates coated with antigen (e.g. recombinant CD98hc, Bsg, or Glut1) are incubated with a sample comprising the antibody and binding of the antibody to the antigen of interest is determined.

Assays for evaluating uptake of systemically administered antibody and other biological activity of the antibody can be performed as disclosed in the examples or as known for the anti-CNS antigen antibody of interest.

In one aspect, assays are provided for identifying anti-CD98hc/BACE1 multispecific antibodies having biological activity. In another aspect, assays are provided for identifying anti-Bsg/BACE1 multispecific antibodies having biological activity. In another aspect, assays are provided for identifying anti-Glut1/BACE1 multispecific antibodies having biological activity. Biological activity may include, e.g., inhibition of BACE1 aspartyl protease activity. Antibodies having such biological activity in vivo and/or in vitro are also provided, e.g., as evaluated by homogeneous time-resolved fluorescence HTRF assay or a microfluidic capillary electrophoretic (MCE) assay using synthetic substrate peptides, or in vivo in cell lines which express BACE1 substrates such as APP.

In another aspect, assays are provided for measuring brain uptake of the antibodies disclosed herein (e.g., anti-CD98hc, Bsg, or Glut1 antibodies). Such assays are described in the Examples below.

In certain embodiments, labeled anti-CD98hc antibodies may be used in the methods disclosed herein. In certain embodiments, labeled anti-Bsg antibodies are provided. In certain embodiments, labeled anti-Glut1 antibodies are provided. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes ³²P, ¹⁴C, ¹²⁵I, ³H, and ¹³¹I, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, β-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

I. Pharmaceutical Formulations

In a further aspect, the invention provides pharmaceutical formulations comprising any of the anti-CD98hc, anti-Bsg, or anti-Glut1 antibodies provided herein, e.g., for use in any of the therapeutic methods described herein. In one embodiment, a pharmaceutical formulation comprises any of the anti-CD98hc, anti-Bsg, or anti-Glut1 antibodies provided herein and a pharmaceutically acceptable carrier (e.g., for use in a therapeutic method disclosed herein). In another embodiment, a pharmaceutical formulation comprises an anti-CD98hc antibody and at least one additional therapeutic agent, e.g., as described below. In another embodiment, a pharmaceutical formulation comprises any of the anti-Bsg or anti-Glut1 antibodies provided herein and at least one additional therapeutic agent, e.g., as described below.

Pharmaceutical formulations of an anti-CD98hc, anti-Glut1, or anti-Bsg antibody (e.g., multispecific antibody or antibody conjugate) as described herein are prepared by mixing such antibody having the desired degree of purity with one or more optional pharmaceutically acceptable carriers, excipients or stabilizers (Remington's Pharmaceutical Sciences 16th edition, Osol, A. Ed. (1980)), in the form of lyophilized formulations or aqueous solutions. Pharmaceutically acceptable carriers, excipients, or stabilizers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredient as necessary for the particular indication being treated, preferably those with complementary activities that do not adversely affect each other. For example, it may be desirable to further provide one or more active ingredients for treating a neuropathy disorder, a neurodegenerative disease, cancer, an ocular disease disorder, a seizure disorder, a lysosomal storage disease, an amyloidosis, a viral or microbial disease, ischemia, a behavioral disorder or CNS inflammation. Exemplary such medicaments are discussed herein below. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in, for example, Remington's Pharmaceutical Sciences 16th edition, Osol, A. Ed. (1980). One or more active ingredients may be encapsulated in liposomes that are coupled to an antibody described herein (see e.g., U.S. Patent Application Publication No. 20020025313).

Sustained-release preparations may be prepared. Suitable examples of sustained-release preparations include semi-permeable matrices of solid hydrophobic polymers containing the antibody, which matrices are in the form of shaped articles, e.g. films, or microcapsules. Non-limiting examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

The formulations to be used for in vivo administration are generally sterile. Sterility may be readily accomplished, e.g., by filtration through sterile filtration membranes.

J. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

It is understood that any of the above articles of manufacture may include an immunoconjugate of the invention in place of or in addition to an anti-CD98hc, anti-Bsg, or anti-Glut1 antibody.

### III. Examples

The following are examples of methods and compositions of the invention. It is understood that various other embodiments may be practiced, given the general description provided above.

A. Materials and Methods

The materials and methods used in the following Examples are described below.

1. Antibody Generation

The Lrp1 extracellular domain (ECD) was expressed in E. coli as His-tagged recombinant protein. The His-tagged CD320 ECD and CD98hc ECD were expressed in Chinese hamster ovary (CHO) cells and the ECD of the murine basigin (mBsg) was expressed as murine Fc tagged protein in CHO cells. All these recombinant proteins were then purified on nickel or protein A columns. The recombinant Lrp8 and InsR were purchased from R&D Systems™ ((catalog #3520-AR-050 and #1544-IR-050, respectively). The recombinant Ldlrad3 was also purchased from Novus Biologicals LLC (Littleton, Colo.) (catalog #H00143458-P01). The anti-Lrp1, anti-InsR, anti-Lrp8 and anti-Ldlrad3 antibodies were generated through naïve phage library sorting methods (described below). The anti-CD320, anti-Bsg, and anti-CD98hc antibodies were generated using the murine extracellular domains of the corresponding proteins to immunize mice, rats, or hamsters using standard protocols. The anti-Glut1 antibody was generated by DNA immunization in mouse using plasmid coding for the full length Glut. The hybridomas that were generated were screened by ELISA for antigen binding and by flow cytometry for recognition of the antigen transiently expressed on HEK cells. All antibodies were reformatted as chimeras containing a human Fc for all studies. Affinities are listed in FIG. 9. Anti-Bsg antibodies A-E are described in the Examples.

2. Naïve Phage Library Sorting

10 μg/mL of the recombinant antigen was coated overnight on NUNC 96 well Maxisorp immunoplates and pre-blocked with PBST [PBS and 1% bovine serum albumin (BSA) and 0.05% Tween 20]. The natural synthetic diversity phage antibody libraries (see, C. V. Lee, et al. J. Mol. Biol. 340, 1073-1093 (2004); and W. C. Liang, et al. J. Mol. Biol. 366, 815-829 (2007)), pre-blocked with PBST, were subsequently added to the plates and incubated overnight at room temperature. The plates were washed with PBST and bound phage were eluted with 50 mM HCl and 500 mM NaCl for 30 minutes and neutralized with 1M Tris base. Recovered phage particles were amplified in E. coli XL-1 Blue cells (Agilent Technologies, Santa Clara, Calif.). During subsequent selection rounds, the incubation time was reduced to 2-3 hours and the stringency of washing was gradually increased. Unique phage antibodies that bind specifically to the antigen were chosen and reformatted to full length IgGs by cloning VL and VH regions of individual clones into the LPG3 and LPG4 vectors, respectively, and transiently expressed in mammalian CHO cells.

3. Development of Antibody Against RMT Targets

Balb/c mice (Charles River Laboratories International, Inc., Wilmington, Mass.), Lewis rats (Charles River, Hollister, Calif.), or Armenian hamsters (Cytogen Research and Development, Inc., West Roxbury, Mass.) were immunized with purified antigen extracellular domain, or DNA encoding the human antigen in the case of Glut1, via footpad or intraperitoneal, at a 3-4 day interval in Ribi adjuvant (Sigma) or plasmid DNA encoding the full length antigen in the presence of GM-CSF diluted in Ringer's solution via hydrodynamic tail vein delivery (HTV), weekly injections. Following 6-12 injections, immune serum titers were evaluated by direct ELISA and FACS binding to transiently transfected 293 cells. Splenocytes and/or lymphocytes from animals demonstrating FACS binding were fused with mouse myeloma cells (X63.Ag8.653; American Type Culture Collection (ATCC®), Manassas, Va., USA) by electrofusion (Hybrimmune™; Harvard Apparatus, Inc., Holliston, Mass.). After 10-14 days, hybridoma supernatants were harvested and screened for IgG secretion by direct ELISA or FACS. Final hybridoma clones demonstrating FACS binding were reformatted into human IgG1 or effectorless, kappa backbone. The reformatted antibodies are expressed and supernatants purified by affinity chromatography using MabSelect SuRe™ (GE Healthcare, Piscataway, N.J.), eluted in 50 mM phosphoric acid, pH 3.0 plus 20×PBS, pH 11 and stored at 4° C.

4. Flow Cytometry Analysis

Purified antibodies were screened on 293 cells transfected with the corresponding antigens. Cells were collected from flasks/dishes, washed with phosphate-buffered saline (PBS), and added to 96-well U-bottom plates (BD Falcon 353077, BD, Franklin Lakes, N.J.) at 1,000,000 cells per well. Samples were added to cells (100 μL/well) and incubated at 4° C. for 30-60 minutes. Plates were then centrifuged (1200 rpm, 5 minutes, 4° C.) and washed twice with PBS/1% FBS (200 μl per well). R-Phycoerythrin-conjugated Ziege anti-human IgG Fc (Jackson ImmunoReseach Laboratories Inc. (West Grove, Pa.); 109-116-098; 100 μl diluted in PBS) was then added and the plates incubated at 4° C. (covered) for 30 minutes. After the final wash, the cells were fixed in PBS containing 1% formalin, and read using a FACSCalibur™ flow cytometer (BD Biosciences, San Jose, Calif.). Mean fluorescence intensity (MFI) of each sample was then measured using the FlowJo software (Treestar, Inc., Ashland, Oreg.).

5. Competition Enzyme-Linked Immunosorbent Assay (ELISA)

Nunc 96-well Maxisorp immunoplates were coated overnight at 4° C. with antigen (2 μg/ml) and blocked for 1 hour at room temperature with blocking buffer PBST. Serial dilutions of bivalent or bispecific antibodies were subsequently added to the plates with a sub-saturating concentration of biotinylated bispecific antibody at room temperature for 1 hour. Plates were washed with wash buffer (PBS with 0.05% Tween 20) and incubated for 30 minutes with horseradish peroxidase (HRP)-conjugated streptavidin, and developed with tetramethylbenzidine (TMB) substrate. Absorbance was measured spectrophotometrically at 650 nm.

6. Radiolabel Trace Dosing

Radioiodination. All antibodies used in the studies were radioiodinated with iodine-125 (¹²⁵I) using the indirect iodogen addition method as previously described (Chizzonite et al., J Immunol, 1991; 147(5):1548-56). The radiolabeled proteins were purified using NAP5™ columns pre-equilibrated in PBS. They were shown to be intact by size-exclusion HPLC.

7. In Vivo Biodistribution in C57BL/6 Female Mice.

All in vivo protocols, housing, and anaesthesia were approved by the Institutional Animal Care and Use Committees of Genentech Laboratory Animal Resources, in compliance with the Association for Assessment and Accreditation of Laboratory Animal Care regulations. Female C57BL/6 mice of about 6-8 weeks of age (17-22 g) were obtained from Charles River Laboratories (Hollister, Calif.). They were administered 5 μCi of the radioiodinated antibodies via IV bolus. At 1, 4, 24, and 48 hours post-dose, blood (processed for plasma), brain, liver, lungs, spleen, bone marrow, and muscle (gastrocnemius) were collected (n=3/antibody) and stored frozen until analyzed for total radioactivity on a gamma counter (2480 Wizard²® Automatic Gamma Counter, PerkinElmer, Waltham, Mass.). The radioactivity level in each sample was calculated and expressed as percentage of Injected Dose per gram or milliliter of sample (% ID/g or % ID/mL). The % ID/g-time data were plotted using GraphPad Prism® (Version 6.05) and the area under the concentration time curve (AUC) was determined. The standard deviations (SD) for the AUC estimates were calculated using the method described by Bailer (Bailer, Journal of Pharmacokinetics and Biopharmaceutics, 1988; 16 (3): 303-309).

8. Immunohistochemistry

Wild-type mice were intravenously injected with 5 mg/kg of antibody followed by PBS perfusion 1 hour post-dose. Brains were drop fixed in 4% paraformaldehyde (PFA) overnight at 4° C., followed by 30% sucrose overnight at 4° C. Brain tissue samples were sectioned at 35 μm thickness on a sliding microtome, blocked for 1-3 hours in 5% BSA, 0.3% Triton, incubated with 1:200 Alexa Fluor® 488 anti-human secondary antibody (Life Technologies, Grand Island, N.Y.) in 1% BSA, 0.3% Triton, for 2 hours at room temperature. Mounted slides were subsequently analyzed by Leica fluorescence microscopy (Leica Microsystems Inc., Buffalo Grove, Ill.).

9. Measuring Antibody Concentrations and Mouse Aβₓ₋₄₀ in Brain and Plasma

The animals' care was in accordance with Genentech IACUC guidelines. All mice used in therapeutic dosing studies were female C57BL/6 wild-type mice, ages 6-8 weeks. Mice were intravenously injected with antibody and taken down at the indicated time post-injection. Prior to perfusion with PBS, whole blood was collected in plasma microtainer tubes (BD Diagnostics, Franklin Lakes, N.J.) and spun down at 14000 rpm for 2 minutes. Plasma supernatant was isolated for antibody and mouse Aβₓ₋₄₀ measurements where appropriate. Brains were extracted and tissues were homogenized in 1% NP-40 (Cal-Biochem, Billerica, Mass.) in PBS containing cOmplete Mini EDTA-free protease inhibitor cocktail tablets (Roche Diagnostics, Indianapolis, Ind.). Homogenized brain samples were rotated at 4° C. for 1 hour before spinning at 14000 rpm for 20 minutes. Supernatant was isolated for brain antibody measurement. For PK/PD studies, one hemi-brain was isolated for Aβₓ₋₄₀ measurements and homogenized in 5M guanidine hydrochloride buffer. Samples were rotated for 3 hours at room temperature prior to diluting (1:10) in 0.25% casein, 5 mM EDTA (pH 8.0) in PBS containing freshly added aprotinin (20 μg/mL) and leupeptin (10 μg/mL). Diluted homogenates were spun at 14000 rpm for 20 minutes, and supernatants were isolated for mouse Aβₓ₋₄₀ measurements.

10. PKAssays

Antibody concentrations in mouse serum and brain samples were measured using an ELISA. NUNC 384 well Maxisorp™ immunoplates (Thomas Scientific, Swedesboro, N.J.) were coated with F(ab′)₂ fragment of donkey anti-human IgG, Fc fragment specific polyclonal antibody (Jackson ImmunoResearch Laboratories, Inc., West Grove, Pa.). After blocking the plates, each antibody was used as a standard to quantify the respective antibody concentrations. Standards and samples were incubated on plates for 2 hours at room temperature with mild agitation. Bound antibody was detected with HRP-conjugated F(ab′)₂ goat anti-human IgG, Fc specific polyclonal antibody (Jackson ImmunoResearch Laboratories, Inc). Concentrations were determined from the standard curve using a four-parameter non-linear regression program. The assay had lower limit of quantitation (LLOQ) values of 3.12 ng/ml in serum and 1.56 ng/ml in brain. For anti-CD98hc brain samples, antibody concentrations in mouse serum and brain samples were measured using an ELISA on the GYROS platform (Gyros Ab, Sweden). Gyros beads are first coated with biotin-conjugated F(ab′)₂ fragment of donkey anti-human IgG, Fc fragment specific polyclonal antibody (Jackson ImmunoResearch Laboratories, Inc). Each antibody was used as a standard to quantify the respective antibody concentrations. Standards and samples were incubated on beads at room temperature following manufacture suggested protocol. Bound antibody was detected with Alexa 647-conjugated F(ab′)₂ goat anti-human IgG, Fc specific polyclonal antibody (Jackson ImmunoResearch). Concentrations were determined from the standard curve using a four-parameter non-linear regression program. The assay had lower limit of quantitation (LLOQ) values of 5 ng/ml in serum and 5 ng/ml in brain.

11. PD Assays

Aβₓ₋₄₀ concentrations in mouse neuronal culture supernatants, plasma and brain samples were measured using an ELISA similar to methods for PK analysis above. Briefly, rabbit polyclonal antibody specific for the C terminus of Aβ₄₀ (Millipore, Bedford, Mass.) was coated onto plates, and biotinylated anti-mouse Aβ monoclonal antibody M3.2 (Covance®, Dedham, Mass.) was used for detection. The assay had LLOQ values of 1.96 pg/ml in plasma and 39.1 pg/g in brain.

12. Primary Mouse Brain Endothelial Cell Isolation

Brain endothelial cells (BEC; CD31+/CD45−) were isolated by FACS from 40 adult female C57B16 mice (6-8 weeks of age). A negatively sorted population (CD31−/CD45−) was collected in parallel for comparison. In total, approximately 5×10⁵ cells were sorted to acquire a BEC population with a purity of ˜92%. Isolated BECs and the negatively selected control cells were lysed in RIPA buffer in the presence of protease inhibitors and separated by SDS-PAGE on a 4-12% Bis-Tris gel. Of the BEC lysate, 10% was used for a silver stained gel, 10% for a Western blot against transferrin receptor (TfR), and the remainder loaded in a single lane and stained with SimplyBlue™ SafeStain Coomassie® (Life Technologies). In parallel lanes adjacent to the BEC lysate, lysates stemming from ˜5000 CD31+/CD45− and ˜40000 CD31−/CD45− cells from the negatively selected population were run for silver staining, anti-TfR Western blot, and Coomassie® staining, respectively.

13. Mass Spectrometry

For mass spectrometry analysis, the Coomassie® stained gel lane corresponding to the BEC lysate (CD31+/CD45−) and the negative control (CD31−/CD45−) lysates were each cut into 15 sections from top to bottom. Each gel lane was subjected to in-gel trypsin digestion using standard methods, essentially as described in Zhang, et al., 2014, Sci Trans Med 34(36): 11929-11947; and in Phu et al., 2011, Mol Cell Proteomics, 10(5):M110. Gel slices were diced into 1 mm cubes and destained by serial washes with 10× gel volumes of 50 mM ammonium bicarbonate, 50% acetonitrile (pH 8.0), then 10× gel volumes 100% ACN for 15 minutes each. In-gel reduction and alkylation were performed with 25 mM dithiothreitol/100 mM ammonium bicarbonate (30 minutes, 50° C.), and 50 mM iodoacetamide/100 mM ammonium bicarbonate (20 minutes, room temperature in the dark), respectively. Gel pieces were subsequently washed and dehydrated with an additional 10× gel volumes of 100% acetonitrile. Trypsin solution was prepared at a concentration of 10 ng/μL trypsin in 50 mM ammonium bicarbonate pH 8.0 with 5% acetonitrile and added to the gel pieces on ice. Gel pieces were soaked in trypsin solution for 1 hour on ice, and in-gel digestion performed overnight at 37° C.). Digested peptides were collected and gel pieces extracted an additional time with 50% acetonitrile/5% formic acid. Samples were dried to completion in a SpeedVac™ and then resuspended in 3% acetonitrile/5% formic acid for analysis.

Peptides were injected onto a 0.1 mm×100 cm C18 column packed with 1.7 μm BEH-130 resin (Waters, Milford Mass.) at a flow rate of 1.5 μl/minute for 10 minutes using a nanoACQUITY UPLC column (Waters). Peptides were separated using a two-stage linear gradient where solvent B (98% acetonitrile/2% water/0.1% formic acid) ramped from 5% to 25% over 20 minutes, and then from 25% to 50% over 2 minutes. Buffer A was comprised of 98% water/2% acetonitrile/0.1% formic acid. Peptides were introduced to an Orbitrap Velos hybrid ion trap-Orbitrap mass spectrometer (ThermoFisher Scientific, San Jose, Calif.) using the ADVANCE Captive Spray Ionization source (Microm-Bruker, Auburn, Calif.). Orbitrap full-MS (MS 1) spectra were collected at 60,000-resolution and used to trigger data dependent MS2 scans in the linear ion trap on the top eight most intense ions. MS2 spectra were searched using Mascot against a concatenated target-decoy database of mouse proteins from UniProt. Peptide spectral matches were sequentially filtered to 5% peptide false discovery rate (pepFDR) using a linear discriminant analysis, and subsequently to a 2% protein false discovery rate (final pepFDR<0.5%). AUC (Area Under Curve) represents the average of two technical replicates for the integrated intensity of the top three most abundant peptide hits as previously described (Ahrne et al., 2013; Proteomics. 17, 2567-2578).

14. In Vivo Two-Photon Microscopy

Wild type mice aged 2-4 months of mixed sex were implanted with cranial windows over the right hemisphere as previously described (Holtmaat et al., 2009; Nat Protoc. 2009; 4(8): 1128-44) and imaged ≧2 weeks post surgery. Mice were anesthetized with sevoflurane (2.5-3% at 0.7 L/minute) during imaging. 100 μL of AngioSense® IVM 680 (Perkin Elmer, Waltham, Mass.) was injected via a tail vein catheter to visualize vasculature and pre-antibody images were acquired by two-photon microscopy. 50 mg/kg of Alexa Fluor® 594 labeled CD98hc/BACE1 antibodies were injected via the tail vein catheter and images were acquired immediately (time 0) and after 6, 24, and 48 hours. The two-photon laser-scanning microscope system (Ultima In Vivo Multiphoton Microscopy System, Prairie Technologies) uses a Ti:sapphire laser (MaiTai® DeepSee™ Spectra Physics) tuned to 860 nm delivering −15 mW to the back-focal plane of a 60× NA 1.1 water immersion objective. Laser power was kept constant across imaging days for each animal. 512×512 pixel resolution stacks of 35-65 μm volumes, in 1 μm z-step sizes were collected for each area.

15. Baculovirus (BV) ELISA

The detailed method was previously described (see, I. Hötzel, et al. mAbs, 4:6, 753-760 (2012)). Briefly, the purified BV particles were immobilized in 384-well ELISA plates (Nunc Maxisorp™) overnight at 4° C. The wells were blocked with blocking buffer (PBS containing 0.5% BSA) for 1 hour at room temperature. After rinsing the plates with PBS, purified antibodies were serially diluted in blocking buffer, 25 μl aliquots were added in duplicate to the ELISA wells and incubated for 1 hour at room temperature. Plates were then washed and 10 ng/mL goat anti-human IgG, (Fcγ-fragment-specific) conjugated to horseradish peroxidase (Jackson ImmunoResearch Laboratories, Inc.) were added to each well. The plates were incubated for 1 hour at room temperature, washed, and then TMB substrate was added to each well. Reactions were stopped after 15 minutes by adding 1 M phosphoric acid to each well. Absorbances were read at 450 nm, referenced at 620 nm. BV score was calculated from the mean of 6 optical density determinations each of which had been normalized by dividing by the average signal observed for non-coated wells.

16. Immunocytochemistry

IMCD3 cells stably overexpressing mouse CD98hc were plated in 384-well optical plates (Perkin Elmer®) and grown for 1-2 days after confluence. Cells were treated for 1 hour at 1 μM with anti-CD98hc bispecific antibodies, washed with PBS, fixed with 4% PFA/4% sucrose/PBS for 5-10 minutes at room temperature (RT), followed by ice cold 100% methanol fixation for 20 minutes. Cells were blocked with 1% donkey serum, 2% BSA, 0.1% Triton-X100 in PBS for 30 minutes at room temperature (RT). Primary antibodies used were mouse anti-Lamp 1 (BD, 1:200) and goat anti-CD98hc (Santa Cruz 1:200), diluted in block, and incubated overnight at 4° C. The following secondary antibodies were used: donkey anti-human IgG Alexa Fluor® 405, donkey anti-goat Cy3, and donkey anti-rabbit Alexa Fluor® 647 (Jackson Immunoresearch).

17. Image Acquisition and Colocalization Analysis

Images were taken on an Opera Phenix™ high content system (Perkin Elmer®) with a 40× NA1.1 water lens in confocal mode. Laser lines 375, 488, 550, and 640 were used. Four images per well were taken and 150-200 cells in each image. Five wells per condition were imaged, thus more than 3000 cells are analyzed per treatment. Images were transferred into ImageXpress® 5.1 for analysis. For each individual channel to be analyzed, the background was removed using the TopHat function and then the “adapted threshold” function was used to create stained object masks over the original channel image for analysis. To quantify only stained intracellular puncta, CD98hc membrane staining was excluded from analysis based on size. Total number of CD98hc puncta was quantified from the entire image consisting of about 150-200 cells. To identify internalized CD98hc staining colocalized with Lamp 1, the “keep marked object” function was used to identify overlapping objects from two different channels. Total number of colocalized CD98hc puncta was quantified from the entire image and sums of each well were reported by the program and exported to Microsoft Excel. Percent CD98hc puncta colocalized was calculated as number of colocalized CD98hc puncta with Lamp 1 divided by the total number of total CD98hc puncta. Averages from 5 wells were calculated and graphed in GraphPad Prism (GraphPad, La Jolla, Calif.).

18. Amino Acid Uptake Assay

IMCD3 cells stably overexpressing CD98hc were plated in 384-well plates (Perkin Elmer®) the day before. Antibodies were added to cells the next morning and incubated for 24 hours at 1 μM in growth media. Four wells per condition were used and the experiment was repeated 3 times. After 24 hours, cells were equilibrated for 30 minutes at 37° C. with Met-Free DMEM. To measure amino acid uptake by the cell, the amino acid methionine analog, homopropargylglycine (HPG, Life Technolgies C10186), was added to the cells at 50 μM final concentration. 10 mM BCH (Sigma) was used as positive control and was added the same time as HPG. After a 30 minute incubation at 37° C., additional growth media was added for another 30 minutes. Cells were then washed with PBS and lysed in RIPA buffer with cOmplete™ protease inhibitors (Roche). All liquid handling was done with an Agilent Bravo automation system (Agilent Technologies, Santa Clara, Calif.) using a 384 tip head. Cell lysates were transferred to 384 well plates and incubated at 4° C. overnight. The transported methionine was detected by biotinylation via the click tag on HPG. Plates were washed 3 times and click reaction was performed according to manufacturer instructions (Life Technologies, Grand Island, N.Y., B10184). The total amount of biotinylated methionine was detected using ELC detection. Results were plotted in GraphPad Prism®.

19. Western Blot Analysis

Mouse brain tissues were isolated after PBS perfusion and homogenized in 1% NP-40 with protease inhibitors as described above (see Measuring antibody concentrations and mouse Aβx-40 in brain and plasma). Approximately 20 μg of protein was loaded onto 4-12% Bis-Tris Novex gels (Life Technologies). Gels were transferred onto nitrocellulose membranes using the iBlot system (Life Technologies) and Western blotting was performed using Odyssey® blocking buffer reagents and secondary antibodies (LI-COR®, Lincoln, Nebr.). Mouse cross-reactive goat anti-CD98hc (Santa Cruz Biotechnology Inc. (Dallas, Tex.), M-20, 1:200) was used to detect CD98hc in brain lysates. Rabbit anti-βactin (Abcam® abcam8227, 1:2000) served as a loading control. Western membranes were imaged and quantified using manufacturer supplied software and system (Odyssey®/LI-COR®).

Wild-type IMCD3 cells were plated in 48-well plates overnight, incubated with antibodies for 24 hours, washed with PBS, and then lysed with RIPA buffer supplemented with cOmplete® protease inhibitors (Roche). Three wells per condition were used and the experiment was repeated 3 times. Lysates were probed for CD98hc with goat anti-CD98hc (Santa Cruz Biotechnology Inc.) and actin (Abcam®) by Western blot as described above.

20. Statistical Analysis

All values are expressed as mean±SEM, unless otherwise indicated, and p-values were assessed by ordinary one-way ANOVA, with Dunnett multiple comparisons test. Correlation analysis between brain TfR and antibody levels was performed using GraphPad Prism® Version 6.

B. Receptor-Mediated Transcytosis Screening of Lrp1 and InsR Revealed Lack of Significant Brain Uptake

This example demonstrates that among the more widely studied receptors for receptor-mediated transport of antibodies across the BBB (TfR, Lrp 1 and InsR), only antibodies against TfR showed significant brain uptake.

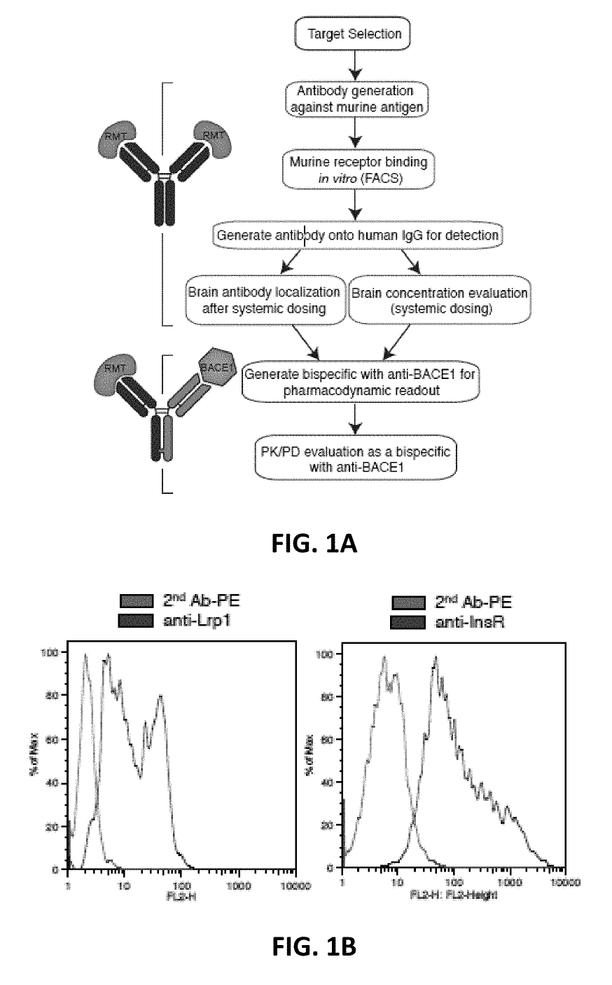

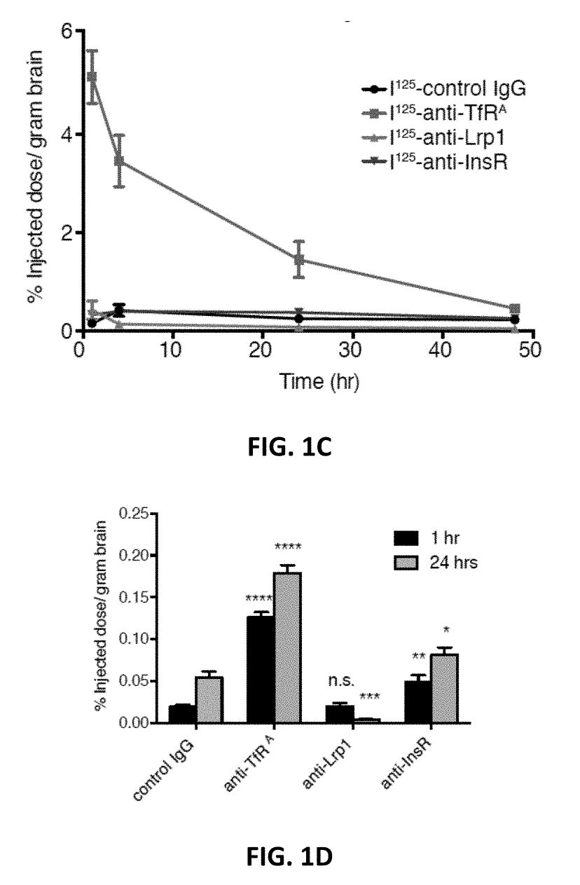

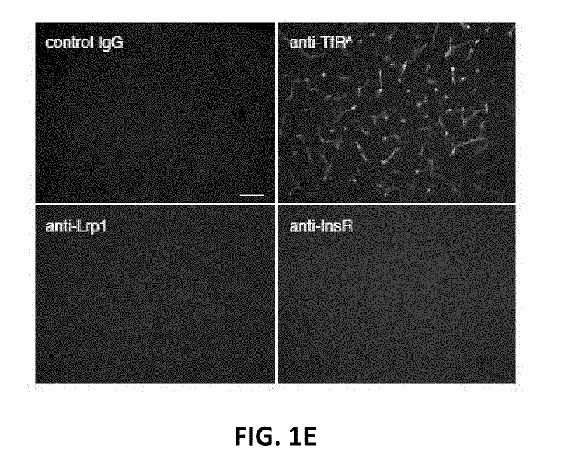

In order to systematically screen antibodies against potential RMT targets for BBB crossing, a general screening cascade was designed involving in vitro confirmation of murine antigen binding prior to systemic in vivo dosing pharmacokinetic studies (FIG. 1A). This method was first used to ascertain whether antibodies against two commonly studied RMT targets, low-density lipoprotein receptor-related protein 1 (Lrp1) and insulin receptor (InsR), could cross the BBB and significantly accumulate in mouse brain. Monoclonal human anti-murine antibodies against Lrp1 and InsR were generated from naïve antibody phage library. Flow cytometric analysis using HEK293 cells expressing murine Lrp1 or murine InsR confirmed positive binding of these antibodies to membrane-displayed targets (FIG. 1B).

To determine whether systemically administered anti-Lrp1 and anti-InsR can be transported into the brain, we assessed brain concentrations of antibodies using trace and therapeutic doses. Both trace and therapeutic doses were investigated, since an inverse relationship between trace and therapeutic doses with the binding affinity to the BBB-R TfR was previously demonstrated. Specifically, high affinity binding to TfR showed robust uptake via trace dosing, but reduced uptake via therapeutic dosing. The opposite was demonstrated for low affinity TfR antibodies. As such, it is concluded that an antibody against an RMT target must work via trace or therapeutic dosing, otherwise the target is likely not viable as a transporter across the BBB.

TfR is a robust RMT target, and antibodies against TfR can cross the BBB and accumulate in brain after systemic administration. Thus, a high affinity anti-TfR antibody (anti-TfR^{A}) was used as a positive control for brain uptake for subsequent trace and therapeutic dosing studies (see, Yu et al., Sci Transl Med. 2011 May 25; 3(84):84ra44; Couch et al., Sci Transl Med. 2013 May 1; 5(183):183ra57, 1-12; Yu et al., Sci Transl Med. 2014 Nov. 5; 6(261):261ra154). A single radiolabeled trace dose of I¹²⁵-control IgG, I¹²⁵-anti-TfR^{A}, I¹²⁵-anti-Lrp1, or I¹²⁵-anti-InsR was intravenously injected into wild-type mice, and radioactivity in brain was measured at various time points post-dose. A significant increase in brain uptake, as measured by percent of injected dose per gram of brain tissue, was observed for I¹²⁵-anti-TfR^{A}, whereas brain uptake of both I¹²⁵-anti-Lrp1 and I¹²⁵-anti-InsR were similar to I¹²⁵-control IgG (FIG. 1C).

It was next asked whether therapeutic doses of these antibodies would result in brain uptake. Wild-type mice were injected with 20 mg/kg (a higher therapeutically relevant dose) of either control IgG, anti-TfR^{A}, anti-Lrp1, or anti-InsR, and brain concentrations of antibody were measured at 1 and 24 hours post-dose following perfusion with PBS. Consistent with previous observations, antibody uptake in brain was observed, but modest, for anti-TfR^{A} at both 1 and 24 hours post-dose compared to control IgG (FIG. 1D). In contrast, no brain accumulation of anti-Lrp1 was observed. Anti-InsR exhibited significant, but modest, increases in brain uptake at both time points.

Immunohistochemical staining of mouse cortical tissue 1 hour after a 5 mg/kg dose revealed pronounced vascular localization of anti-TfR^{A}, whereas no antibody localization was observed for anti-Lrp1 or anti-InsR, indicating a lack of localization of systemically administered antibodies targeting Lrp1 and InsR on brain endothelial cells (FIG. 1E). These results show that, of these widely studied receptors, only antibodies against TfR exhibited robust brain uptake.

C. Gene (mRNA) Enrichment at the BBB is not Sufficient for Antibody Transport to the CNS

This Example demonstrates that gene (mRNA) enrichment at the BBB is not a sufficient criterion for determining whether a plasma membrane receptor expressed on brain endothelial cells is a successful RMT target for antibody transport across the BBB.

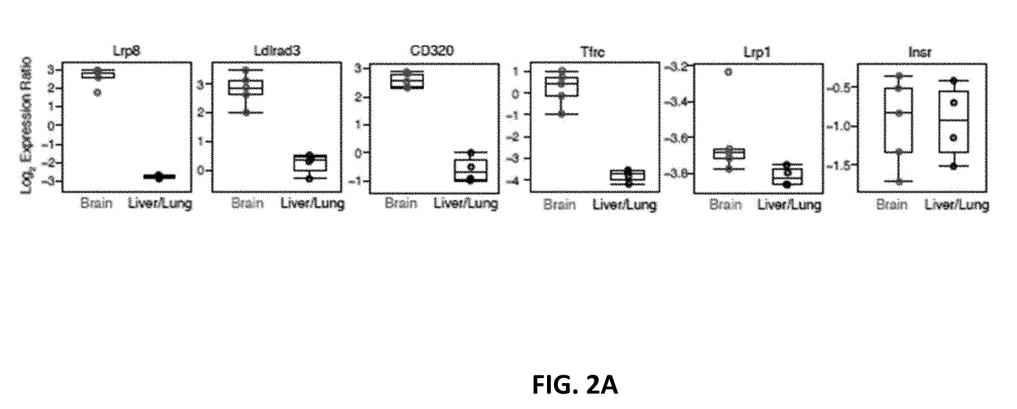

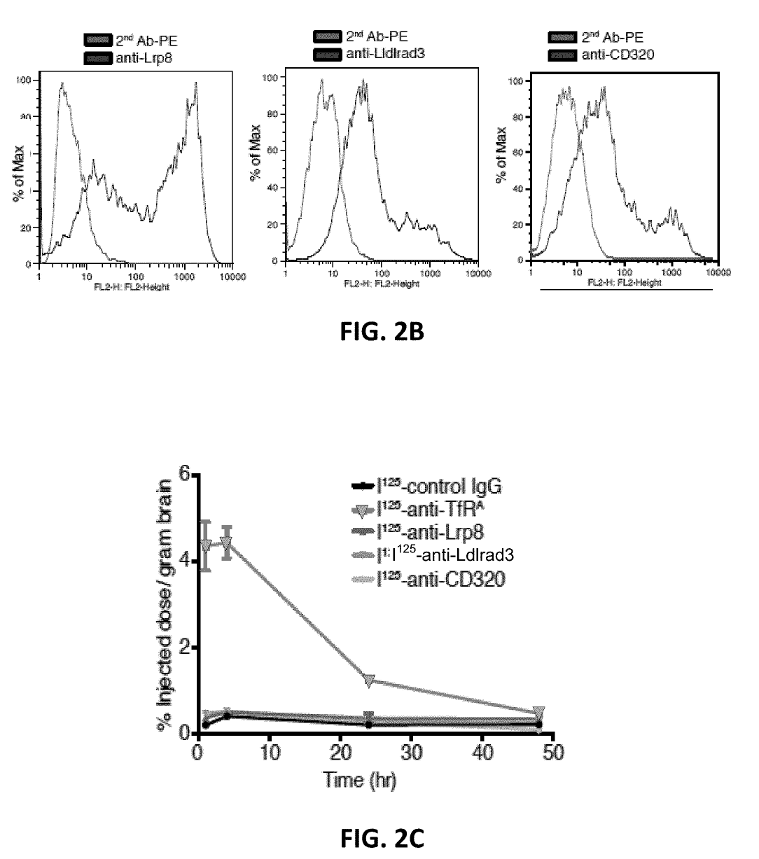

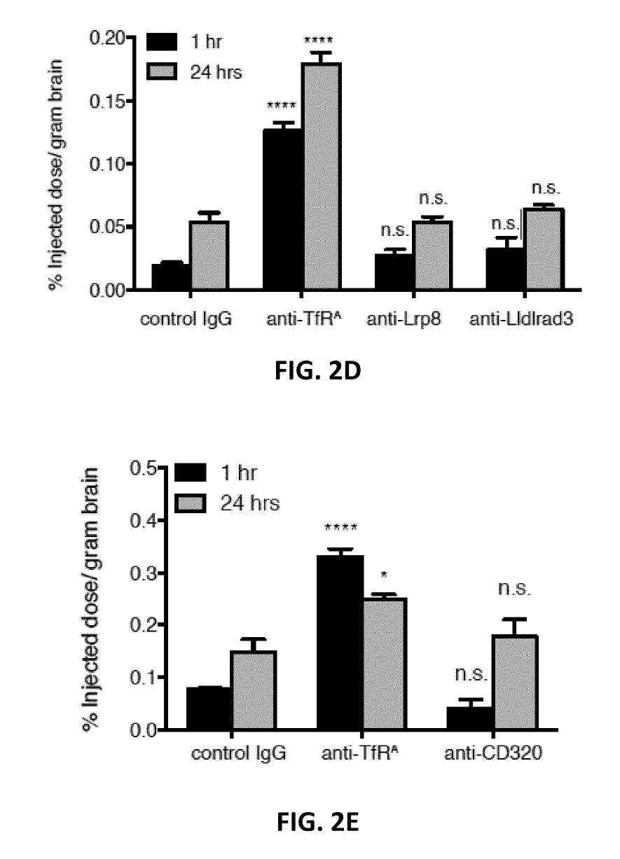

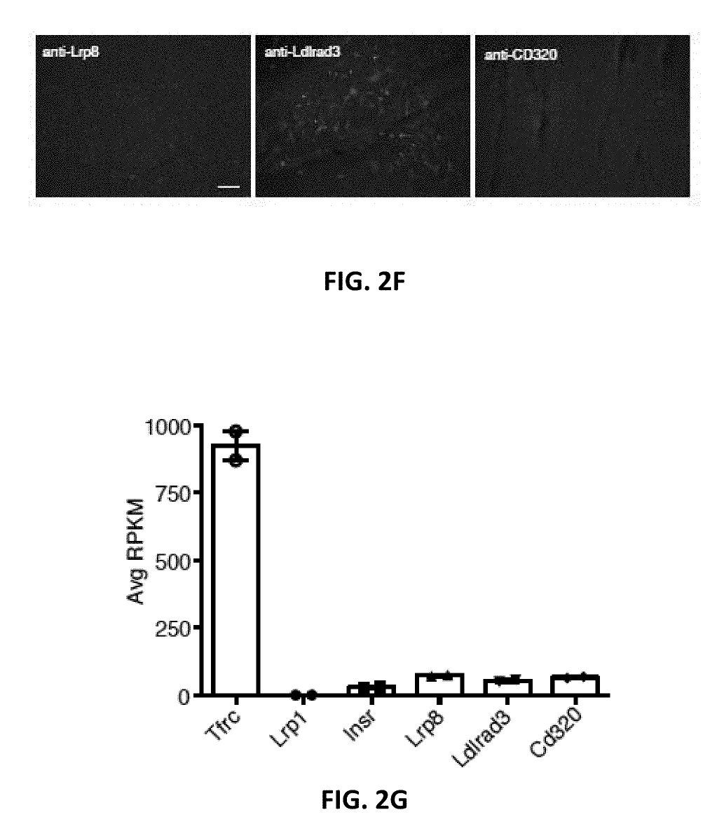

Ideally, RMT targets would be highly expressed at the BBB but have lower expression in peripheral organs. This property may improve safety and antibody pharmacokinetics by reducing target-mediated clearance in organs other than the brain. Previously, genes enriched at the BBB were identified using microarray expression profiling of FACS-purified endothelial cells compared to liver and lung endothelial cells from wild-type mice (Tam et al., 2012, Devt. Cell 22:403-417). Several candidate genes coding for single-pass transmembrane receptors were identified as potential RMT targets based on their high enrichment at the BBB: Lrp8, Ldlrad3, and CD320 (FIG. 2A). Interestingly, while higher mRNA expression at the BBB was observed for Tfrc, neither Lrp1 nor Insr showed higher BBB expression compared to liver and lung endothelial cells, suggesting these commonly studied RMT targets lacked enrichment at the BBB.

To determine whether antibodies targeting products of genes that are enriched at the BBB would result in significant antibody uptake, monoclonal anti-murine antibodies against Lrp8, Ldlrad3, and CD320 were generated. Flow cytometry analysis using HEK293 cells expressing murine antigen confirmed positive binding for all three antibodies (FIG. 2B). A single radiolabel trace dose of I¹²⁵-control IgG, I¹²⁵-anti-TfR^{A}, I¹²⁵-anti-Lrp8, I¹²⁵-anti-Ldlrad3, or I¹²⁵-anti-CD320 was intravenously injected into wild-type mice. Of the injected antibodies, only I¹²⁵-anti-TfRA exhibited significant uptake in brain, whereas I¹²⁵-anti-Lrp8, I¹²⁵-anti-Ldlrad3, and I¹²⁵-anti-CD320 showed similar brain levels as I¹²⁵-control IgG (FIG. 2C). Similar results were observed when wild-type mice were intravenously dosed at a therapeutically relevant dose of 20 mg/kg (FIGS. 2D and 2E). Immunohistochemical staining of cortical brain tissue 1 hour after a 5 mg/kg dose reveals a lack of antibody localization at the BBB for anti-Lrp8 and anti-CD320, while anti-Ldlrad3 showed modest immunoreactivity (FIG. 2F).

Although the microarray analysis identified Lrp8, Ldlrad3, and CD320 mRNA expression to be highly enriched on brain endothelial cells, antibodies against these transmembrane receptors failed to cross the BBB to any appreciable amount. Recently, Zhang et al. (2014, supra) made available a dataset with a comprehensive RNA-seq transcriptome analysis of distinct cell populations in the mouse brain, including brain vascular endothelial cells providing quantitative mRNA expression data (accessible at web.stanford.edu/group/barres_lab/brain_rnaseq.hmtl). Applicant's examination of the failed RMT target candidates within this dataset revealed low absolute mRNA expression of Lrp8, Ldlrad3, and CD320 on brain endothelial cells (FIG. 2G) and Lrp1 and Insr showed very low mRNA expression in this cell population. In contrast, transcript levels of Tfrc were ˜12-fold (compared to Lrp8) to ˜500-fold (compared to Lrp1) higher than the other candidate genes in brain endothelial cells (FIG. 2G). This analysis shows that in the absence of antibody brain uptake experiments, mRNA expression data alone were insufficient to identify suitable RMT targets.

D. Proteomic Identification of Highly Expressed Transmembrane Proteins at the BBB

This Example describes the identification, using a proteomics approach, of plasma membrane proteins that are highly expressed at the BBB and that could be potential novel RMT targets.

Although relative transcript levels of Ldlrad3 and CD320 were selectively enriched in brain endothelial cells (BECs), it was hypothesized that their absolute protein expression level at the BBB may be a limiting factor preventing any potential antibody uptake across the BBB, as suggested by the poor brain immunohistochemical staining. In order to investigate whether absolute protein level would better predict potential RMT receptors, a proteomics approach was employed to identify transmembrane proteins that are highly expressed in brain endothelial cells.

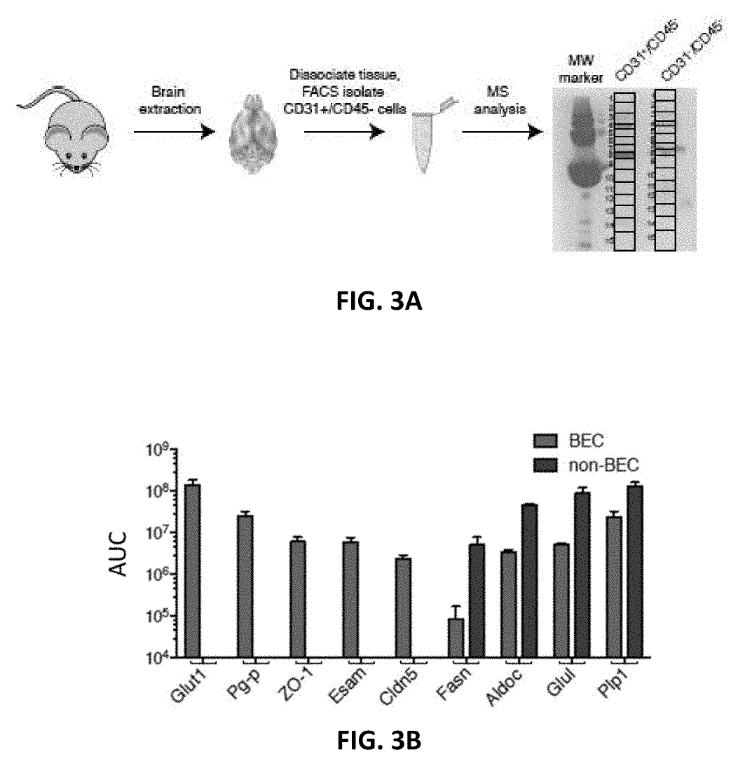

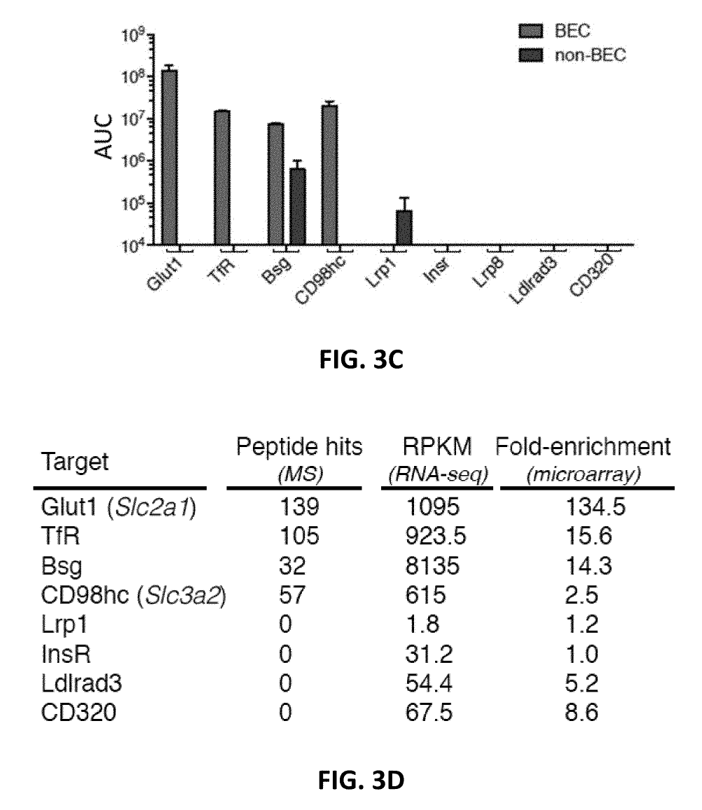

Similar to the methods previously described for gene expression profiling of the BBB vasculature (Tam et al. 2012, supra), flow cytometry was used to isolate CD31-positive and CD45-negative brain endothelial cells (BECs) from wild-type mice (FIG. 3A). Mass spectrometry (MS) analysis of flow cytometry-purified BECs was verified by identification of previously characterized endothelial cell-specific proteins such as Pg-p, Glut1, ZO-1, and Esam (FIG. 3B). Peptide counts from the negatively selected non-BEC lysate (i.e., CD31-negative/CD45-negative) revealed an abundance of glial-specific proteins (Fasn, Aldoc, Glu1, Plp1).

Consistent with its robust RMT properties, TfR was found to be abundantly expressed in the BEC population (FIG. 3C). In fact, peptide counts revealed TfR to be the highest single-pass transmembrane protein in the BEC population. Consistent with mRNA expression, protein levels of Lrp1, InsR, Lrp8, Ldlrad3, and CD320 were below detection, although some peptide counts of Lrp1 were detected in the non-BEC population (FIG. 3C).

These results are therefore consistent with the results above demonstrating lack of significant uptake of antibodies targeting previously described RMT targets (Lrp1 and InsR), and targets with preferential gene expression in brain endothelial cells compared to liver/lung mRNA (Lrp8, Ldlrad3 and CD320).

Importantly, this proteomics analysis revealed several highly abundant transmembrane proteins that have not previously been studied as antibody targets for RMT across the BBB. These include the glucose transporter Glut1 (FIG. 3B), the extracellular matrix metalloproteinase inducer basigin (CD147) (FIG. 3C), and the solute carrier CD98 heavy chain (FIG. 3C). These RMT targets are also enriched at the BBB based on microarray expression and RNA sequencing profiling data (FIG. 3D), and thus were chosen as potential new RMT candidate targets for further investigation.

E. Brain Uptake of Antibodies Against Basigin

This Example describes characterization of anti-basigin antibody uptake into the brains of wild-type mice.

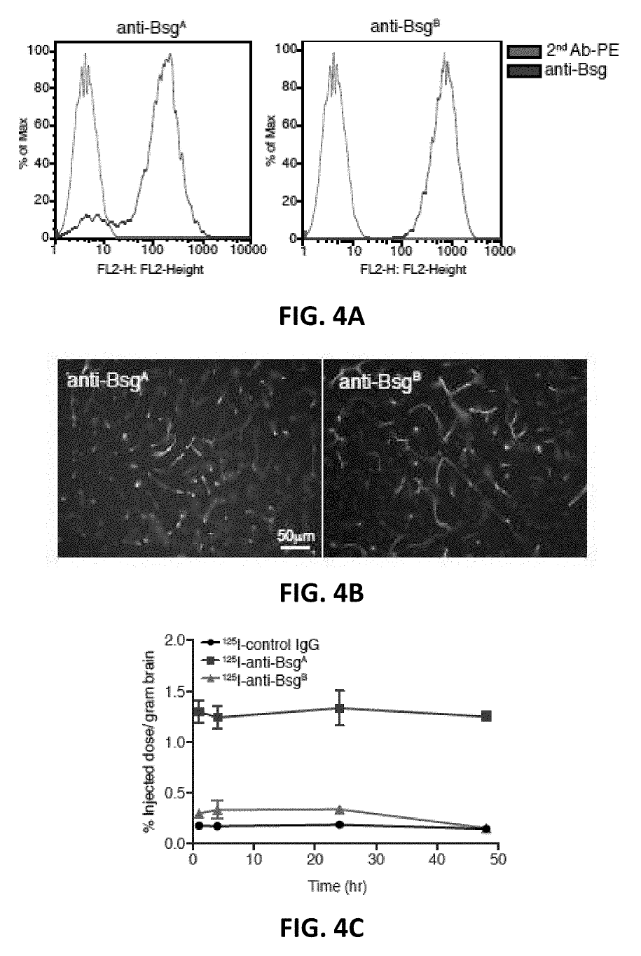

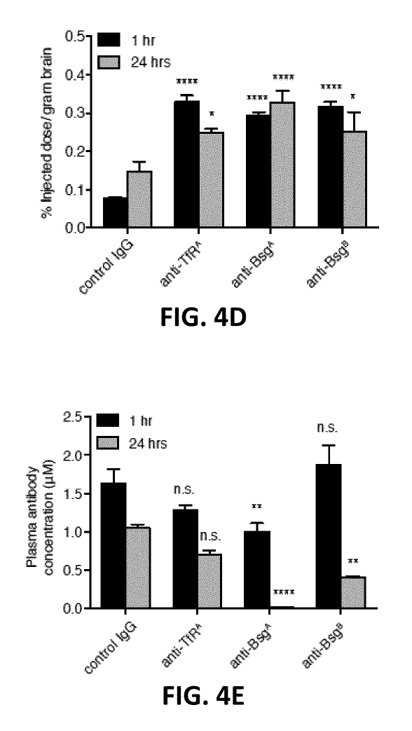

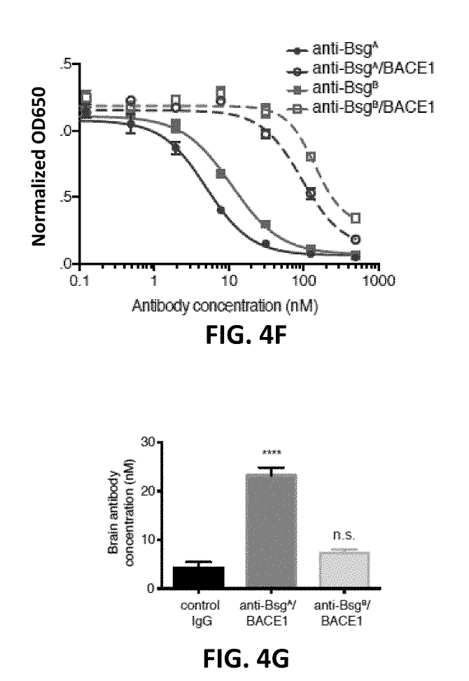

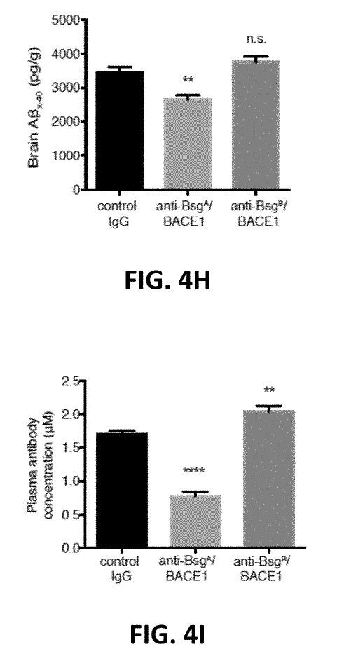

Monoclonal antibodies against basigin were generated via mouse immunization with the extracellular domain of the murine basigin protein and a series of antibody clones were purified, identified herein as anti-Bsg^{A}, anti-Bsg^{B}, anti-Bsg^{C}, anti-Bsg^{D}, and anti-Bsg^{E}. Binding of anti-Bsg^{A} and anti-Bsg^{B} to the target was confirmed by flow cytometry using HEK293 cells transiently expressing murine basigin (FIG. 4A). To determine whether these antibodies bind basigin in vivo, wild-type mice were intravenously injected with 5 mg/kg of either anti-Bsg^{A} or anti-Bsg^{B}. Immunohistochemical staining of mouse cortical tissue 1 hour post-dose revealed pronounced vascular localization of both anti-Bsg^{A} and anti-Bsg^{B}, similar to what was previously observed with anti-TfR^{A} (FIG. 4B, compared to FIG. 1E). Three additional clones, anti-Bsg^{C}, anti-Bsg^{D}, and anti-Bsg^{E}, were also tested and produced similar results as anti-Bsg^{A} and anti-Bsg^{B}It was next explored whether these antibodies can be taken up into brain by testing both trace and therapeutic dosing paradigms. A single radiolabel trace dose of I¹²⁵-control IgG, I¹²⁵-anti-Bsg^{A}, or I¹²⁵-anti-Bsg^{B} was systemically administered into wild-type mice. A significant increase of I¹²⁵-anti-Bsg^{A} was observed in the brain for the duration of the study, while there was a modest increase in I¹²⁵-anti-Bsg^{B} compared to I¹²⁵-control IgG (FIG. 4C). Three additional clones, Bsg^{C}, Bsg^{D}, and Bsg^{E}, were also tested. No significant brain uptake by trace dosing was observed for the additional clones.

Next, a more therapeutically relevant dose of 20 mg/kg was used to test whether basigin antibodies can accumulate in the brain. Wild-type mice were intravenously injected with 20 mg/kg of control IgG, anti-TfR^{A}, anti-Bsg^{A}, or anti-Bsg^{B}, and antibody concentrations in plasma and brain were determined at 1 and 24 hours post-dose. Compared to control IgG, brain concentrations of both Bsg antibodies were significantly higher at both time points (FIG. 4D). Brain uptake of anti-Bsg clones was compared to that of anti-TfR^{A}. Brain concentrations of anti-Bsg^{B} were similar to anti-TfR^{A}, but concentrations of anti-Bsg^{A} were significantly higher at 24 hours post-dose compared to brain concentrations of anti-TfR^{A}.

To further examine transport of anti-Bsg across the BBB into the brain parenchyma, a bispecific anti-Bsg/BACE1 antibody was generated that binds to both Bsg and the amyloid precursor protein (APP) cleavage enzyme beta-secretase (BACE1) (Atwal et al., Sci Transl Med. 2011 May 25; 3(84):84ra43). BACE1 is considered to be the primary contributor of amyloid beta (AP3) formation found in plaques in the brains of Alzheimer's disease patients (Vassar et al., Science. 1999 Oct. 22; 286(5440):735-41). Similar to Applicant's previous approach with anti-TfR/BACE1, the bispecific anti-Bsg/BACE1 allows for a pharmacodynamic readout of antibody crossing the BBB into the brain parenchyma (Yu et al. 2011, supra; Atwal et al 2011, supra). Affinities for the bivalent (monospecific) anti-Bsg and bispecific (monovalent anti-Bsg) anti-Bsg/BACE1 antibodies were determined by competitive ELISA. All antibodies showed a decrease in basigin binding affinity in the bispecific (monovalent) format. See FIG. 4F.

To assess brain uptake of these bispecific antibodies, wild-type mice were intravenously injected with a single 50 mg/kg dose of control IgG, anti-Bsg^{A}/BACE1, or anti-Bsg^{B}/BACE1. At 24 hours post-dose, there was a significant increase in antibody concentration in brains of mice injected with anti-BsgA/BACE1 compared to control IgG (FIG. 4G). This increase in brain concentration of anti-Bsg^{A}/BACE1 correlated with a ˜23% reduction in brain Aβ levels (FIG. 4H). In contrast, mice treated with anti-Bsg^{B}/BACE1 did not show an increase in antibody concentration in brain and, as expected, no Aβ reduction was observed. Anti-Bsg^{D}/BACE1 also showed significant uptake, however significant uptake of Bsg^{C}/BACE1 and BsgE/BACE1 was not observed.

While anti-BsgA/BACE1 and anti-Bsg^{B}/BACE1 have similar monovalent binding affinity, the bivalent (monospecific) Bsg antibodies differed in the extent of brain uptake in both the trace and therapeutic dosing paradigms (FIGS. 4C and 4D). These antibodies bind distinct epitopes based on competition data, which could play a role in Bsg trafficking and transport at the BBB and account for the observed difference in brain uptake. To determine whether any of the antibodies show non-specific binding to cell membranes which could contribute to target-independent tissue uptake, an ELISA assay was used to determine off-target binding to baculovirus (BV) particles (Hotzel et al., MAbs. 2012 November-December; 4(6):753-60). Anti-Bsg^{B} had a BV score within the normal range as did anti-Bsg^{C}, anti-Bsg^{D}, and anti-Bsg^{E}, while anti-Bsg^{A} exhibited high BV particle binding. Furthermore, much faster clearance was observed of both the bivalent and bispecific anti-Bsg^{A} compared to anti-Bsg^{B} (FIGS. 4E and 4I).

F. Brain Uptake of Antibodies Against Glut1

This Example describes the characterization of anti-Glut antibody uptake at the BBB as monovalent and bispecific antibody.

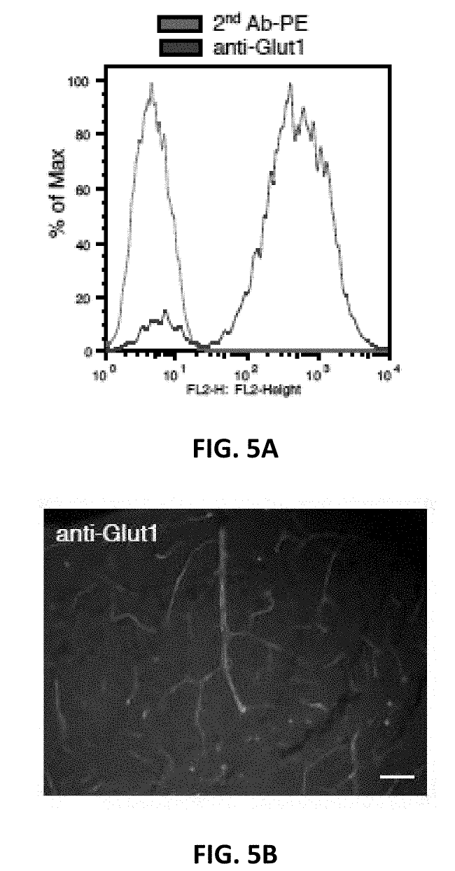

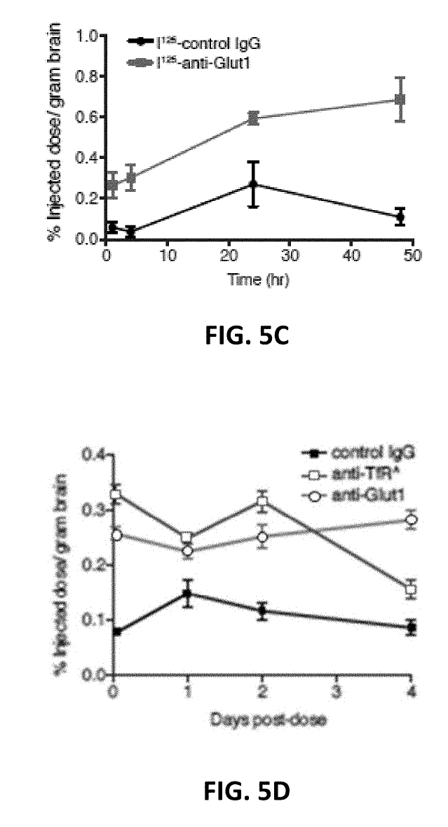

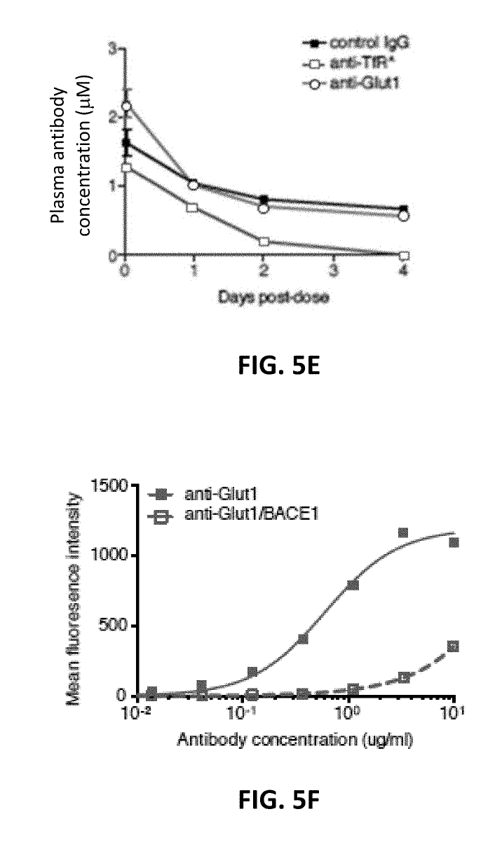

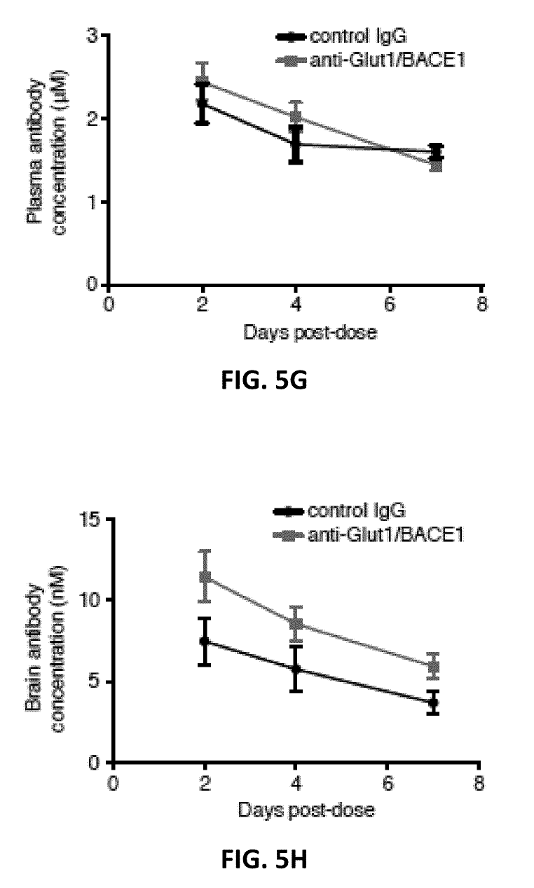

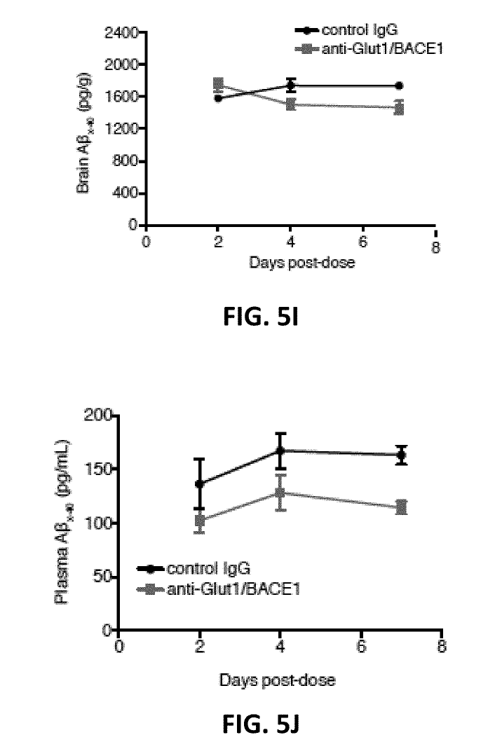

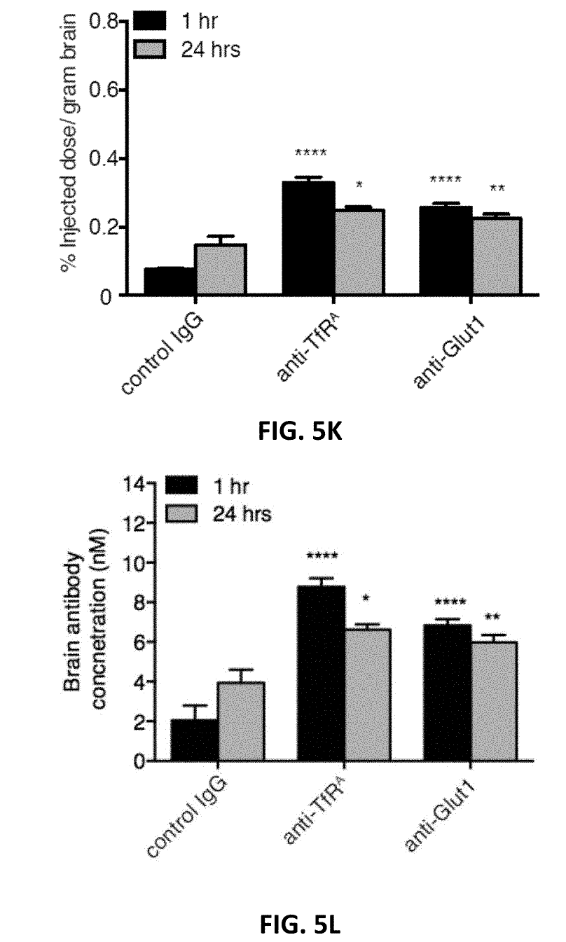

Although multi-pass receptors are not commonly considered for RMT, both high enrichment and protein expression of the glucose transporter Glut1 at the BBB (see FIGS. 3B and 3D) merited evaluation of this plasma membrane protein as a potential transport target. A monoclonal antibody against Glut1 was generated via immunization with the hGlut1 cDNA. Positive antigen binding was confirmed by flow cytometry using HEK293 cells transiently expressing murine Glut1 (FIG. 5A).

To determine whether this antibody binds Glut1 in vivo, wild-type mice were intravenously injected with 5 mg/kg of anti-Glut1. Immunohistochemical staining of mouse cortical tissue 1 hour post-dose revealed vascular localization of anti-Glut1 (FIG. 5B).

It was next explored whether anti-Glut1 can be taken up into the brain in both trace and therapeutic dosing paradigms. A single radiolabel trace dose of either I¹²⁵-control IgG or I¹²⁵-anti-Glut1 was intravenously injected into wild-type mice. A significant increase of I¹²⁵-anti-Glut1 was observed in the brain compared to I¹²⁵-control IgG at all time points post-dose (FIG. 5C). Because brain concentrations of I¹²⁵-anti-Glut1 appeared to steadily increase over time in the trace dosing study, it was decided to extend the time course of the therapeutic dosing study to include 2 and 4 days post-dose time points. When dosed at 20 mg/kg, brain concentrations of anti-Glut1 were comparable to anti-TfR^{A} at 1 and 2 days post-dose, but reached a much higher brain concentration at 4 days post-dose (FIG. 5D). In a similar experiment using a 20 mg/kg dose of anti-Glut1, brain concentrations of anti-Glut1 were ˜1.5-3 fold higher than control IgG and comparable to anti-TfR^{A} at both time points (FIG. 5K and FIG. 5L).

In both trace and therapeutic dosing paradigms, a distinct difference was observed in the pharmacokinetics of anti-Glut1 uptake in brain compared to anti-TfR^{A}. Whereas concentrations of anti-TfR^{A} peaked either hours (at trace doses, see FIG. 1C and FIG. 2C) or around 1 day (at therapeutic doses) post-dose, brain concentrations of anti-Glut1 increased over time (FIGS. 5C and 5D). This can be attributed, at least in part, to a much slower clearance rate of anti-Glut1 in the periphery compared to anti-TfR^{A} (FIG. 5E), and is consistent with the enrichment of Glut1 expression at the BBB compared to peripheral tissues (FIG. 3C). Together, these data suggest that not only is there significant brain uptake of anti-Glut1 after systemic injection, but that it also has desirable pharmacokinetic properties.

To determine whether anti-Glut1 is transported across the BBB into the brain parenchyma, a bispecific anti-Glut1/BACE1 antibody was generated. Glut1 binding affinity was significantly reduced in the monovalent/bispecific anti-Glut1/BACE1 antibody, as assessed by flow cytometry (FIG. 5F). Because the monovalent anti-Glut1 showed peripheral pharmacokinetics similar to that of control IgG (FIG. 5E), a more extensive PK/PD study with the bispecific antibody was performed.

Wild-type mice were intravenously injected with a single 50 mg/kg dose of either control IgG or anti-Glut1/BACE1 and brain and plasma concentrations of antibody were determined at 1, 2, 4, and 7 days post-dose. Anti-Glut1/BACE1 showed comparable pharmacokinetics compared to control IgG, similar to what was observed with the bivalent antibody (FIG. 5G). A modest increase in brain uptake of anti-Glut1/BACE1 was observed at all time points post-dose (FIG. 5H).

Consistent with the limited extent of antibody accumulation in brain, a small reduction in Abeta was observed (FIG. 5I). Full function of the anti-BACE1 arm was confirmed by significant reduction in plasma Abeta (FIG. 5J). Together, these data provide evidence that the bivalent anti-Glut1 can cross the BBB and significantly accumulate in brain.

G. Brain Uptake of Antibodies Against CD98hc

This Example describes the characterization of anti-CD98hc antibody uptake at the BBB as a bivalent (monospecific) and as a bispecific antibody.

One of the highest single-pass transmembrane protein hits from the proteomics dataset was the solute carrier CD98hc. To determine whether high expression of CD98hc at the BBB enables large molecule transport, two CD98hc antibodies (anti-CD98hc^{A} and anti-CD98hc^{B}) were generated.

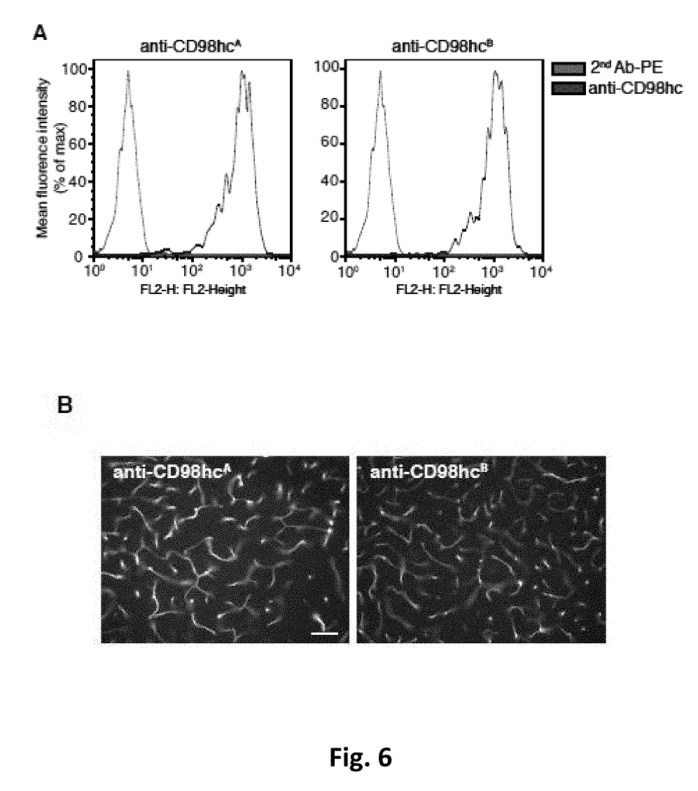

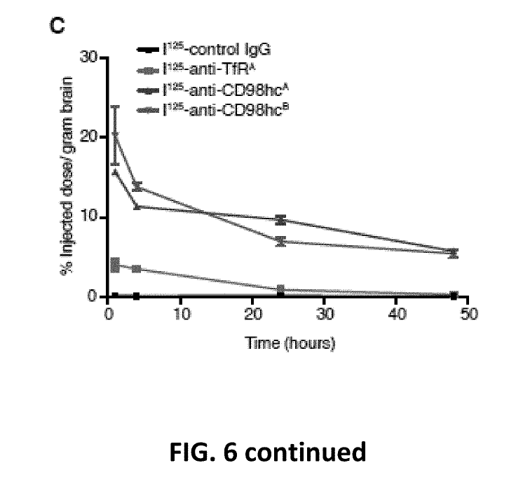

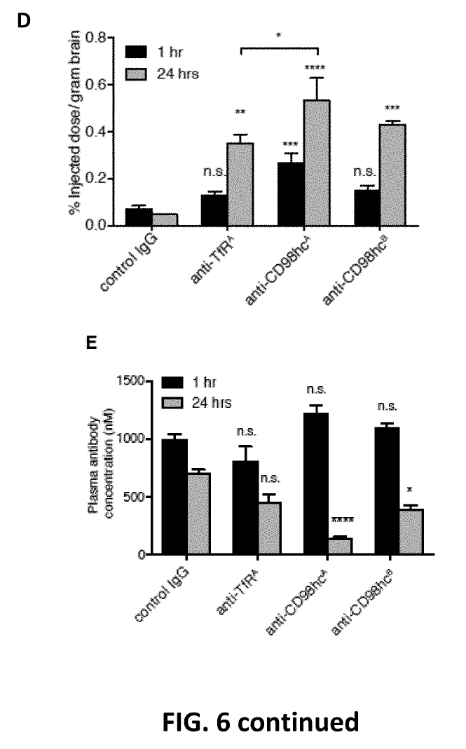

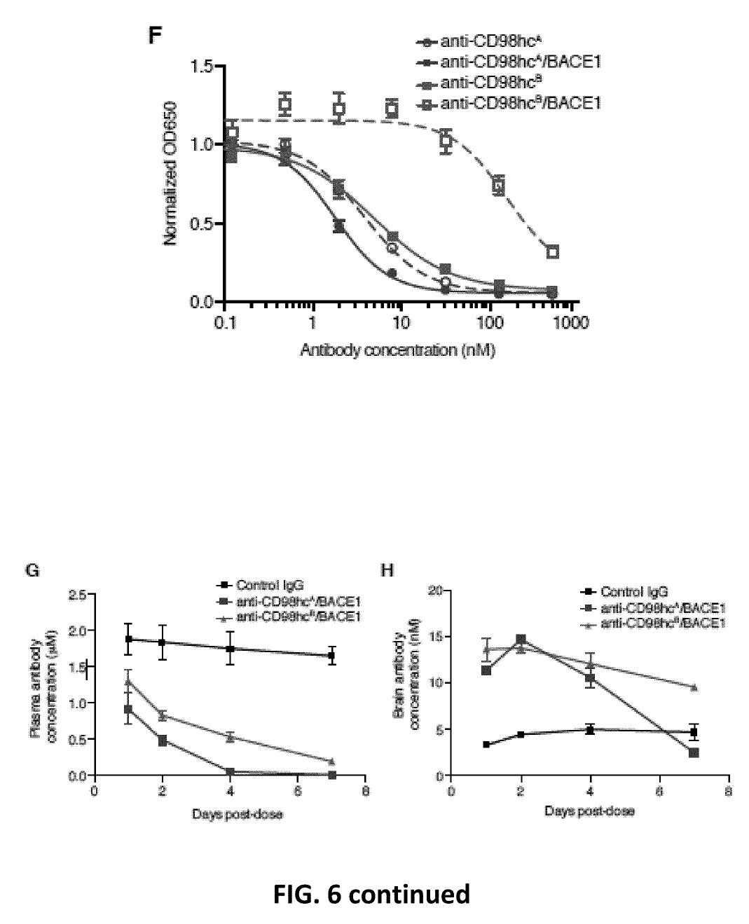

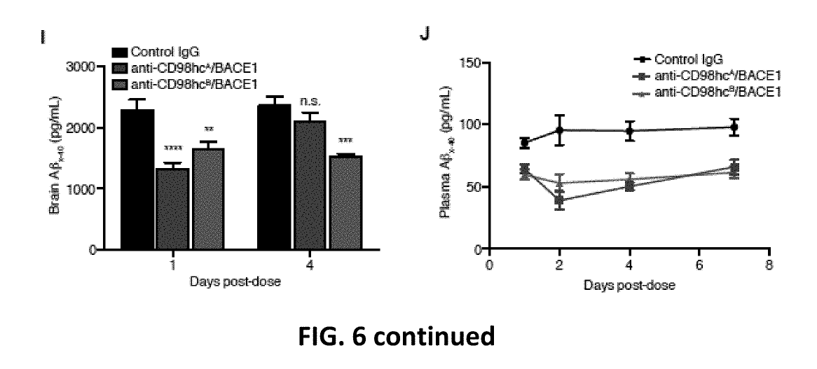

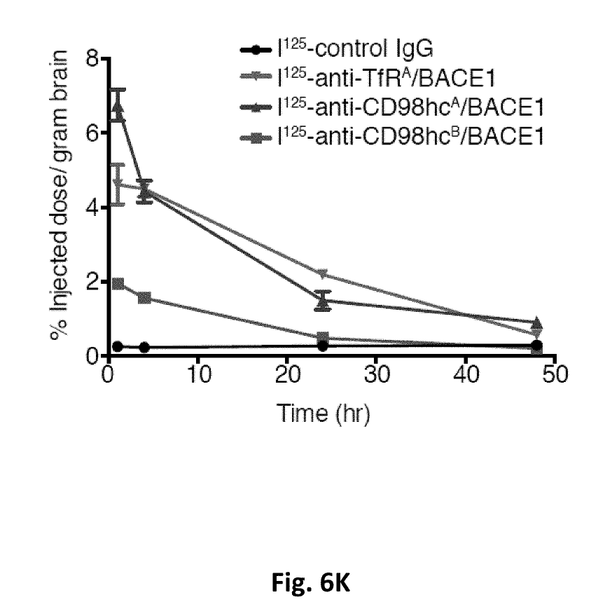

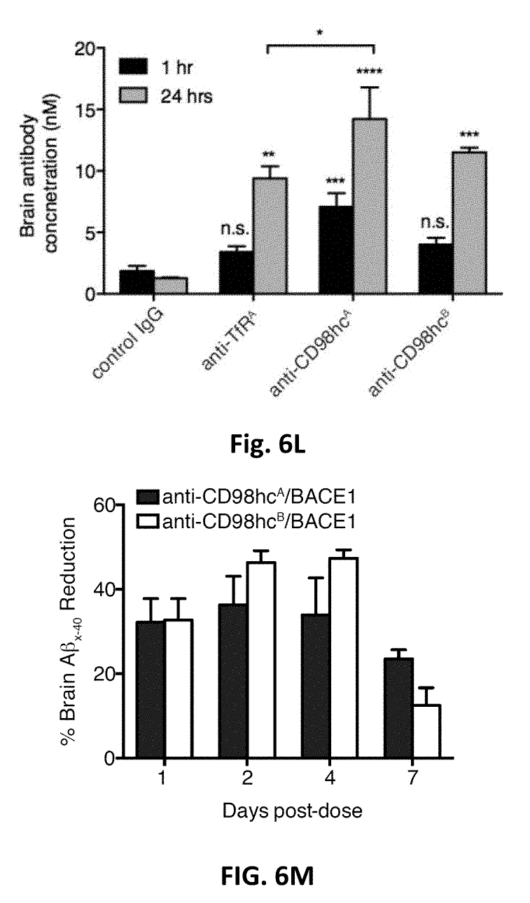

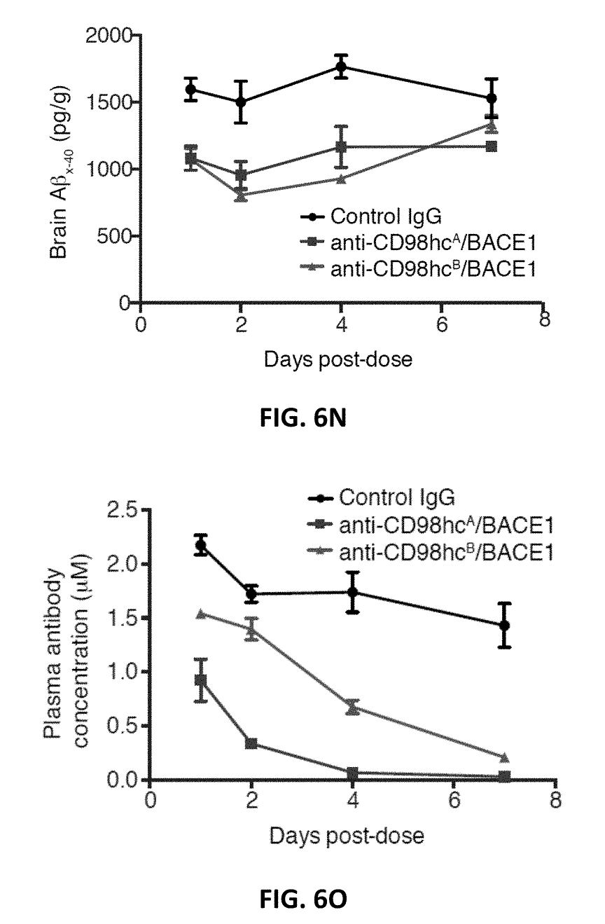

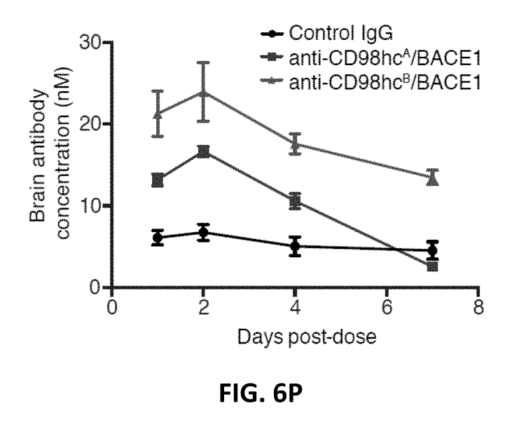

Flow cytometry analysis using HEK293 cells stably expressing murine CD98hc confirmed that both antibodies bound to murine CD98hc (FIG. 6A). A 5 mg/kg intravenous injection of both anti-CD98hc^{A} and anti-CD98hc^{B} resulted in pronounced vascular staining in brain tissue of mice (FIG. 6B). The binding affinities of the CD98hc antibodies are shown in FIG. 9.

It was next explored whether these antibodies could be taken up into the brain. A single radiolabel trace dose of I¹²⁵-control IgG, I¹²⁵-anti-TfRA, I¹²⁵-anti-CD98hcA, or I¹²⁵-anti-CD98hc^{B} was intravenously injected into wild-type mouse. Strikingly higher brain levels were observed for both I¹²⁵-anti-CD98hcA and I¹²⁵-anti-CD98hc^{B} compared to both control IgG and I¹²⁵-anti-TfRA (FIG. 6C). Notably, the extent of brain uptake at trace doses of I¹²⁵-anti-CD98hc was ˜4-5 fold higher than I¹²⁵-anti-TfRA at peak concentrations. When administered at a therapeutic dose of 20 mg/kg, significant brain uptake was also observed for both anti-CD98hc^{A} and anti-CD98hc^{B} at 24 hours post-dose, comparable to what was observed with anti-TfR^{A} (FIG. 6D).

Plasma concentration of antibody showed an enhanced clearance of anti-CD98hc^{A}, and modest clearance of anti-CD98hc^{B} at 24 hours post-dose (FIG. 6E). Target-independent clearance did not seem to contribute to the faster clearance, as assessed by baculovirus particle binding but is instead likely due to expression of CD98hc on peripheral cells (see, Parmacek et al., Nucleic Acids Res. 1989 Mar. 11; 17(5):1915-31; Nakamura et al., J Biol Chem. 1999 Jan. 29; 274(5):3009-16).

Of the three RMT candidates (CD98hc, Glut1 and Bsg), systemic injections of CD98hc antibodies revealed the highest brain concentrations. Brain concentrations of anti-CD98hc^{A} and anti-CD98hc^{B} were ˜9 and 11-fold over that of control IgG, respectively, at 24 hours post-dose (FIGS. 6D and 6L). Furthermore, at 24 hours, brain levels of anti-CD98hc^{A} were significantly higher than that of anti-TfRA. Although all three RMT candidates showed brain uptake by trace and therapeutic dosing, these in vivo studies reveal CD98hc to be the most robust RMT candidate relative to Bsg and Glut1 based on the higher brain concentrations achieved in both trace and therapeutic dosing paradigms.

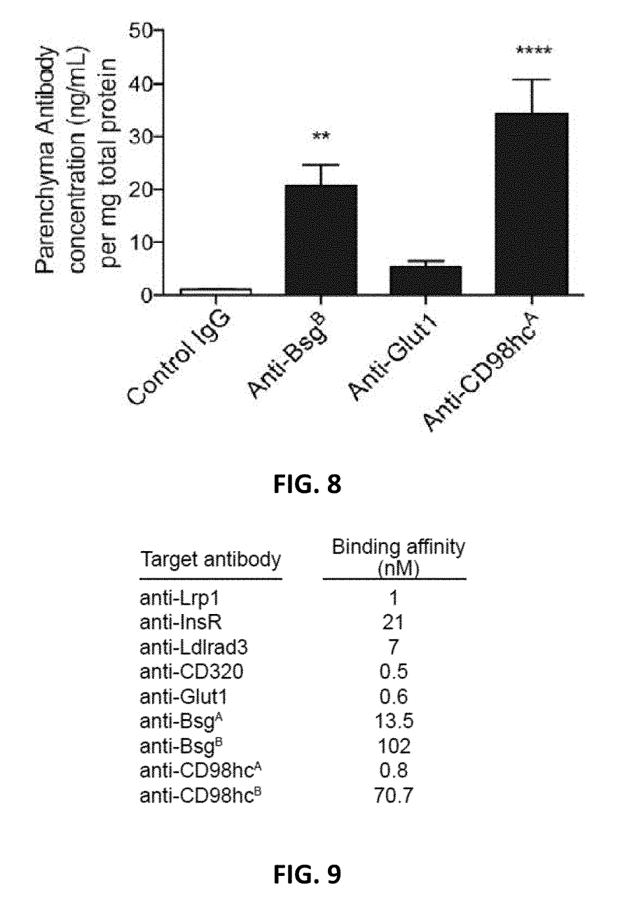

To further confirm that dosed antibodies definitively cross the BBB and penetrate parenchyma, the amount of antibody retained in the parenchyma fraction after microvessel depletion of brain homogenates was assessed by ELISA. Dosed antibody was clearly detected for all three targets compared to the control antibody, suggesting there was significant passage of antibody across the BBB which bound to the parenchyma isolates (FIG. 8). Consistent with trace and therapeutic dose studies, anti-CD98hc antibody in the parenchyma fraction showed the greatest brain concentration (FIG. 8). The minimal uptake of anti-Glut1 may be a consequence of the specific expression of Glut1 (Slc2a1) in brain endothelial cells. Unlike CD98hc (Slc3a2) that is also expressed in microglia and astrocytes, the protocol to deplete microvessels may not allow for an accurate quantification of remaining antibody in the parenchymal fraction where no antigen is expressed.

Nevertheless, antibodies against CD98hc were selected for further in vivo validation as bispecific antibodies as a result of multiple lines of evidence showing the most robust uptake in brain.

H. Generation of Bispecific Anti-CD98hc/BACE1 Antibodies

This Example describes the generation and characterization of bispecific antibodies that bind to CD98hc and BACE1.

To determine whether anti-CD98hc is transported across the BBB into the brain parenchyma, two bispecific anti-CD98hc/BACE1 antibodies were generated. The bispecific antibodies bound to CD98hc on one arm, and to the amyloid precursor protein (APP) cleavage enzyme 3-secretase (BACE1) on the other arm. BACE1 is an enzyme that is considered to be the primary generator of brain 3-amyloid (AP) found in plaques in the brains of Alzheimer's disease patients. An antibody against BACE1 has been designed to inhibit enzymatic activity and thereby reduce AP production (Atwal et al., 2011). However, this antibody has poor BBB penetration and is thus ineffective at reducing brain AP unless it is either dosed at very high concentrations, or paired with anti-TfR as a bispecific antibody. Both anti-CD98hc^{A} and anti-CD98hc^{B} were reformatted as bispecific antibodies to allow for a direct pharmacodynamic measure of antibody accumulation in brain as a result of CD98hc-mediated transport across the BBB into the parenchyma through the measurement of brain AP levels. Affinities for the bivalent anti-CD98hc and bispecific anti-CD98hc/BACE1 antibodies were determined by competitive ELISA. A modest loss in anti-CD98hc^{A} binding affinity was observed in the monovalent (i.e., bispecific) format, while a more significant shift in affinity was observed for anti-CD98hc^{B} (FIG. 6F). While the affinity of anti-CD98hc^{A} was reduced only ˜2-fold, the affinity of anti-CD98hc^{B} was reduced by ˜100-fold, indicating that avidity plays an important role in the bivalent binding of this particular antibody. Radiolabel trace dosing revealed significantly higher peak brain uptake at 1 hour post-dose of anti-CD98hc^{A}/BACE1 compared to both control IgG and anti-TfRA/BACE1 (FIG. 6K, P<0.0001). The lower affinity anti-CD98hc^{B}/BACE1 exhibited increased brain uptake compared to control IgG but was below that of anti-CD98hc^{A}/BACE1, likely due to the substantial reduction in binding affinity to CD98hc as a bispecific antibody. To determine extent and duration of anti-CD98hc/BACE1 brain uptake and pharmacodynamic response, a single 50 mg/kg intravenous injection of either control IgG or anti-CD98hc/BACE1 was administered in wild-type mice. As a result of target-mediated clearance, pharmacokinetics of the higher affinity anti-CD98hc^{A}/BACE1 was faster compared to the lower affinity anti-CD98^{B}/BACE1 in the plasma (FIG. 6G and FIG. 6O).

In brain, there was a significant increase in uptake of both CD98hc/BACE1 antibodies compared to control IgG at 1, 2, and 4 days post-dose (FIG. 6H and FIG. 6P). At 7 days post-dose, brain concentrations of the lower affinity anti-CD98hc^{B}/BACE1 remained elevated, while brain concentration of the higher affinity anti-CD98hc^{A}/BACE1 was comparable to control IgG, presumably due to the loss in exposure in the periphery. Taken together, the lower affinity anti-CD98hc^{B}/BACE1 produced better peripheral and brain exposure over time compared to the higher affinity anti-CD98hc^{A}/BACE1 (FIGS. 6G and 6H). Interestingly, this inverse relationship between antibody affinity and duration of brain exposure was also previously observed for anti-TfR/BACE1 antibodies (Couch et al., 2013, supra). Both CD98hc/BACE1 bispecific antibodies significantly reduced brain Aβ levels 1 day post-dose, which remained reduced at 4 days post-dose with anti-CD98hc^{B}/BACE1 treatment (FIG. 6I), which was indicative of successful transport of these antibodies into the brain parenchyma (see also FIG. 6M and FIG. 6N). Plasma AP3 remained significantly reduced across all time points (FIG. 6J). Together, these data provide robust evidence for CD98hc as a novel RMT target for brain uptake of antibody therapeutics across the BBB.

In vivo two-photon microscopy was also performed to visualize in real time the trafficking of fluorescently labeled CD98hc/BACE1 bispecific variants within the parenchyma and subcortical vasculature of therapeutically dosed mice. Compared to mice dosed with control IgG and anti-CD98hc^{B}/BACE1, a distinct difference in the vascular clearance of anti-CD98hc^{A}/BACE1 was detected, as predicted by the faster plasma pharmacokinetics of the higher affinity variant. In addition, greater diffuse signal in the parenchyma of mice dosed with fluorescently labeled anti-CD98hc^{B}/BACE1 by 48 hours, and to a lesser extent anti-CD98hc^{A}/BACE1 was observed, indicating enhanced crossing of the antibody through the BBB.

I. Antibody Treatments do not Alter Endogenous CD98hc Expression and Function

This Example demonstrates that CD98hc is a novel high capacity RMT pathway capable of delivering antibody therapeutics across the BBB without perturbing CD98hc biology.

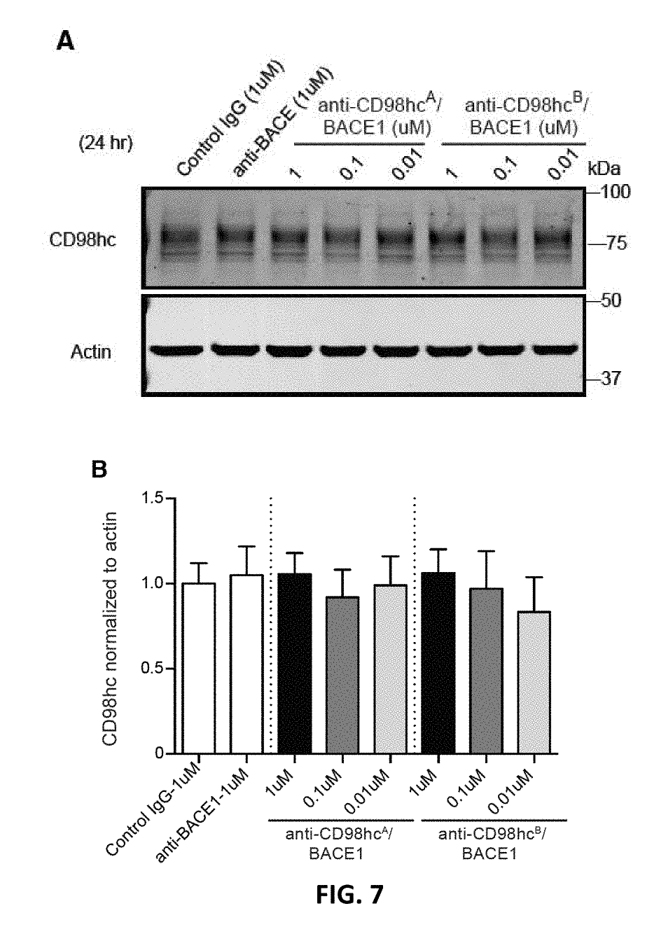

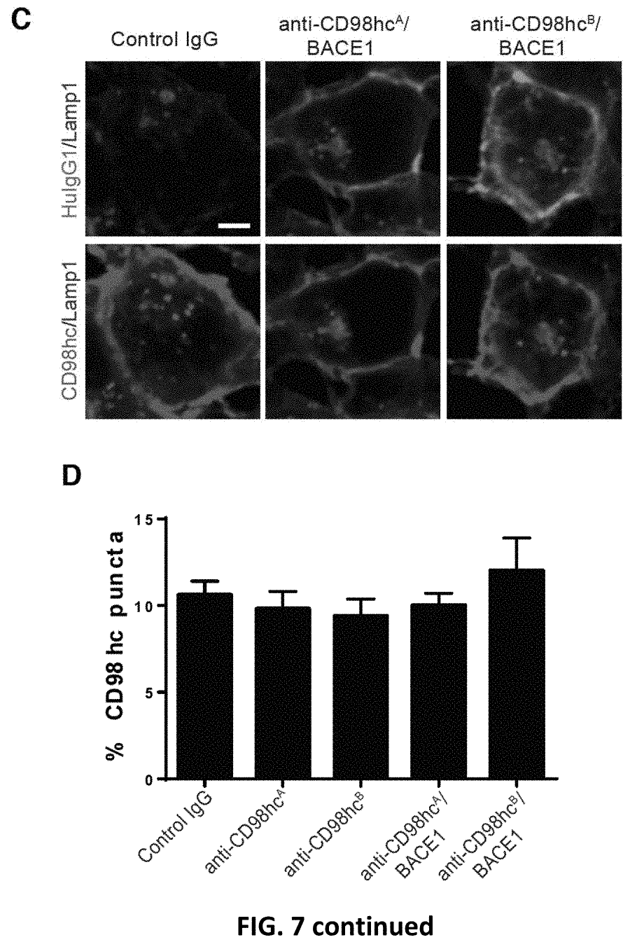

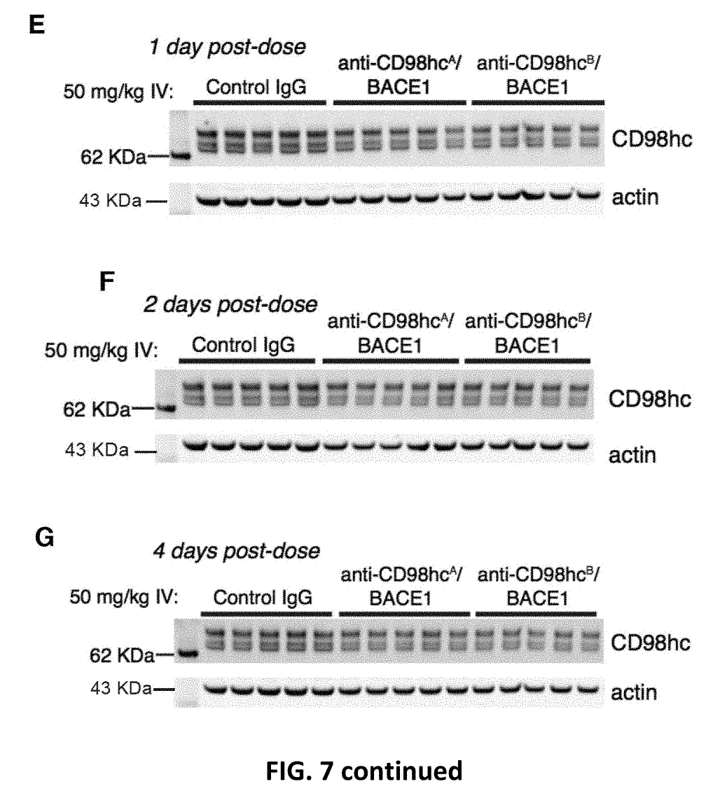

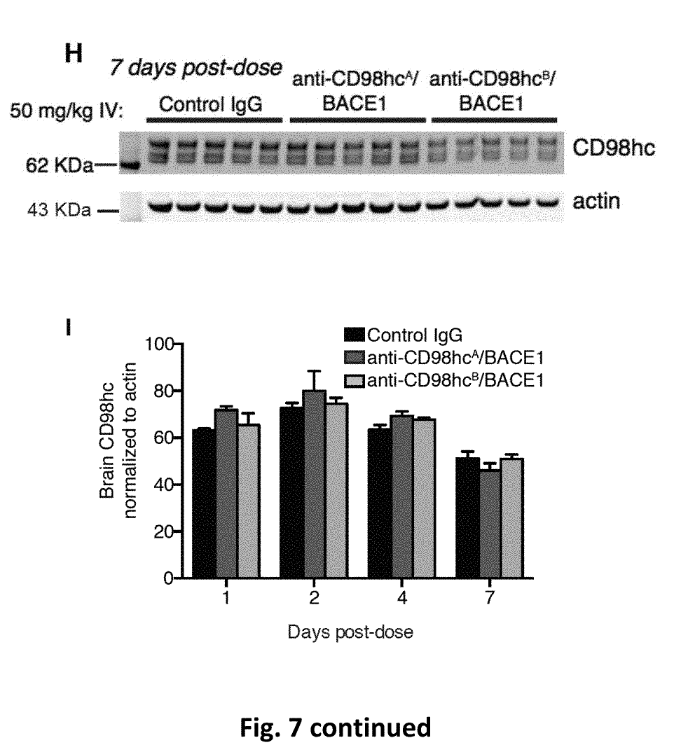

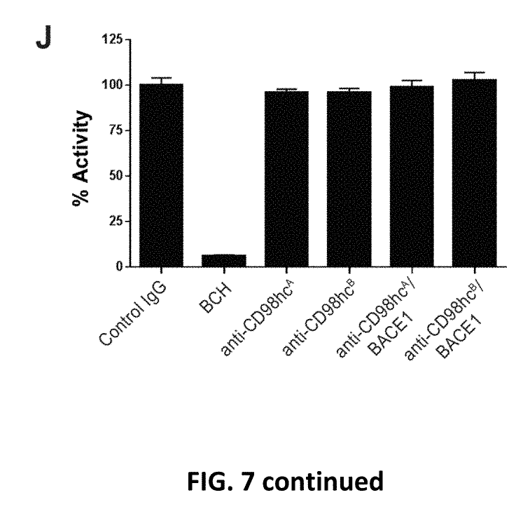

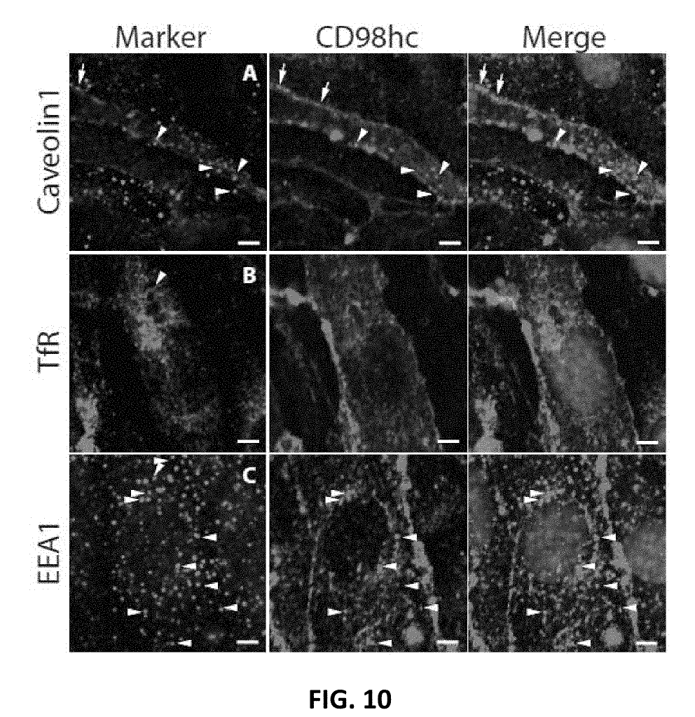

Immunocytochemistry on primary mouse brain endothelial cells revealed that a majority of CD98hc localized to the plasma membrane with some colocalization with caveolin1- and EEA1-positive puncta (FIG. 10). Very few puncta colocalized with TfR, a marker of recycling endosomes. It was previously observed that antibodies against TfR drive lysosomal degradation of TfR in an affinity-dependent manner, leading to decreased TfR levels both in vitro and in vivo. Thus, the endogenous levels of CD98hc were examined in IMCD3 cells (barrier-forming mouse kidney epithelium with uniform CD98hc expression levels) treated with control antibody or anti-CD98hc bispecific variants. Incubation with increasing concentrations of anti-CD98hc bispecific antibodies did not change the expression level or stability of CD98hc (FIGS. 7A and 7B). Furthermore, it was also examined whether antibody treatment induced changes in the subcellular localization of CD98hc. Consistent with the Western blot results, a majority of CD98hc remained on the plasma membrane and increased trafficking of CD98hc to Lamp 1-positive lysosomes was not observed (FIGS. 7C and 7D). Moreover, neither CD98hc bispecific affinity variant affected total brain CD98hc expression in brain lysates from mice that were dosed with 50 mg/kg of anti-CD98hc/BACE1 between 1 and 7 days (FIGS. 7E-7I).

The CD98hc amino acid transport level in the presence or absence of the anti-CD98hc antibodies was also evaluated. As a positive control, transport inhibition by the system-L-specific substrate BCH (2-amino-2-norbornane-carboxylic acid) was observed. No inhibition was observed with anti-CD98hc antibody treatments (FIG. 7J). Taken together, these data indicate that CD98hc is a novel high capacity RMT pathway capable of delivering antibody therapeutics across the BBB without perturbing CD98hc biology. Plasma Aβ remained significantly reduced across all time points (FIG. 7J).

The following Table provides sequences referenced herein.

| SEQ | | |
|---|---|---|
| ID | | |
| NO: | Description | Sequence |
| 1 | anti-BsgA | EIVLTQSPATMPASPGEKVTLTCRASSSIRYIYWYQQKSGT |
| | light chain | SPKLWIYDTSKLASGVPNRFSGSGSGTSYSLTISSMETEDT |
| | variable | ATYYCQQGRSYPLTFGSGTKLEIK |
| | domain | |
| | polypeptide | |
| 2 | anti-BsgA | EVQLVESGGGLVLPGRSMKLSCAASGFTFRTYYMAWVRQ |
| | heavy chain | APTKGLEWVASISIGGDNTYYRDSVMGRFTISRDDAKSTL |
| | variable | HLQMDNLRSEDTATYYCVRLRGYFDYWGQGVMVTVSS |
| | domain | |
| | polypeptide | |
| 3 | anti-BsgA LC | RASSSIRYIY |
| | CDR1 | |
| 4 | anti-BsgA LC | DTSKLAS |
| | CDR2 | |
| 5 | anti-BsgA LC | QQGRSYPLT |
| | CDR3 | |
| 6 | anti-BsgA HC | GFTFRTYYMA |
| | CDR1 | |
| 7 | anti-BsgA HC | SISIGGDNTYYRDSVMG |
| | CDR2 | |
| 8 | anti-BsgA HC | VRLRGYFDY |
| | CDR3 | |
| 9 | anti-BsgA LC | EIVLTQSPATMPASPGEKVTLTC |
| | FR1 | |
| 10 | anti-BsgA LC | WYQQKSGTSPKLWIY |
| | FR2 | |
| 11 | anti-BsgA LC | GVPNRFSGSGSGTSYSLTISSMETEDTATYYC |
| | FR3 | |
| 12 | anti-BsgA LC | FGSGTKLEIK |
| | FR4 | |
| 13 | anti-BsgA HC | EVQLVESGGGLVLPGRSMKLSCAAS |
| | FR1 | |
| 14 | anti-BsgA HC | WVRQAPTKGLEWVA |
| | FR2 | |
| 15 | anti-BsgA HC | RFTISRDDAKSTLHLQMDNLRSEDTATYYC |
| | FR3 | |
| 16 | anti-BsgA HC | WGQGVMVTVSS |
| | FR4 | |
| 17 | anti-BsgB | NTVMTQSPTSMFISVGDRVTMNCKASRSVGTNVDWYQQ |
| | Light chain | KTGQSPTLLFYGASNRYIGVPDRFTGSGSGTDFTLTISNMQ |
| | variable | AEDLAVYYCLQYNYNWAFGGGTKLELK |
| | domain | |
| | polypeptide | |
| 18 | anti-BsgB | EVQLVESGGGLVQPGRSLKLSCVASGFTFNNYWMTWIRQ |
| | heavy chain | APGKGLEWFASITNTGGSTYYPDSVKGRFTISRDNAQSTL |
| | variable | YLQTNSLRPEDTATYYCARRDGSYYPYYWYFDLWGPGTT |
| | domain | VTVSS |
| | polypeptide | |
| 19 | anti-BsgB LC | KASRSVGTNVD |
| | CDR1 | |
| 20 | anti-BsgB LC | GASNRYI |
| | CDR2 | |
| 21 | anti-BsgB LC | LQYNYNWA |
| | CDR3 | |
| 22 | anti-BsgB HC | GFTFNNYWMT |
| | CDR1 | |
| 23 | anti-BsgB HC | SITNTGGSTYYPDSVKG |
| | CDR2 | |
| 24 | anti-BsgB HC | ARRDGSYYPYYWYFDL |
| | CDR3 | |
| 25 | anti-BsgB LC | NTVMTQSPTSMFISVGDRVTMNC |
| | FR1 | |
| 26 | anti-BsgB LC | WYQQKTGQSPTLLFY |
| | FR2 | |
| 27 | anti-BsgB LC | GVPDRFTGSGSGTDFTLTISNMQAEDLAVYYC |
| | FR3 | |
| 28 | anti-BsgB LC | FGGGTKLELK |
| | FR4 | |
| 29 | anti-BsgB HC | EVQLVESGGGLVQPGRSLKLSCVAS |
| | FR1 | |
| 30 | anti-BsgB HC | WIRQAPGKGLEWFA |
| | FR2 | |
| 31 | anti-BsgB HC | RFTISRDNAQSTLYLQTNSLRPEDTATYYC |
| | FR3 | |
| 32 | anti-BsgB HC | WGPGTTVTVSS |
| | FR4 | |
| 33 | anti-BsgC | DIQMTQSPASLSASLGETVSIECLASEGISNSLAWYQQKPG |
| | Light chain | KSPQLLIYGASSLQDGVPSRFSGSGSGTQFSLKISGMQPED |
| | variable | EGIYYCQQGYKYPFTFGSGTKLEIK |
| | domain | |
| | polypeptide | |
| 34 | anti-BsgC | EVQLVESGGSLVQPGRSMKVSCAASGFTFTKYYMAWVR |
| | heavy chain | QAPTKGLEWVASISTGGGNTYYRDSVKGRFTISRDNAKST |
| | variable | LYLQMDSLRSEDTATYYCARTLINYSDYADYVMDAWGQ |
| | domain | GASVTVSS |
| | polypeptide | |
| 35 | anti-BsgC LC | LASEGISNSLA |
| | CDR1 | |
| 36 | anti-BsgC LC | GASSLQD |
| | CDR2 | |
| 37 | anti-BsgC LC | QQGYKYPFT |
| | CDR3 | |
| 38 | anti-BsgC HC | GFTFTKYYMA |
| | CDR1 | |
| 39 | anti-BsgC HC | SISTGGGNTYYRDSVKG |
| | CDR2 | |
| 40 | anti-BsgC HC | ARTLINYSDYADYVMDA |
| | CDR3 | |
| 41 | anti-BsgC LC | DIQMTQSPASLSASLGETVSIEC |
| | FR1 | |
| 42 | anti-BsgC LC | WYQQKPGKSPQLLIY |
| | FR2 | |
| 43 | anti-BsgC LC | GVPSRFSGSGSGTQFSLKISGMQPEDEGIYYC |
| | FR3 | |
| 44 | anti-BsgC LC | FGSGTKLEIK |
| | FR4 | |
| 45 | anti-BsgC HC | EVQLVESGGSLVQPGRSMKVSCAAS |
| | FR1 | |
| 46 | anti-BsgC HC | WVRQAPTKGLEWVA |
| | FR2 | |
| 47 | anti-BsgC HC | RFTISRDNAKSTLYLQMDSLRSEDTATYYC |
| | FR3 | |
| 48 | anti-BsgC HC | WGQGASVTVSS |
| | FR4 | |
| 49 | anti-BsgD | DIQMTQSPASLSASLGETVSIECLASEGISNSLAWYQQKPG |
| | Light chain | KSPQLLIYDASSLQVGVPSRFSGSGSGTQYSLKISGLQPEDE |
| | variable | GVYYCQQGYKYPFTFGSGTKLEIK |
| | domain | |
| | polypeptide | |
| 50 | anti-BsgD | EVQLVESGGGLVQPGRSMKLSCAASGFTLSNYYMAWVR |
| | heavy chain | QAPTKGLEWVASISTGGGYTYYRDSVKGRFTISRDLAKST |
| | variable | LYLQMDSLRSEDTATYHCARSLINYRNYGDYVMDAWGQ |
| | domain | GASVTVSS |
| | polypeptide | |
| 51 | anti-BsgD LC | LASEGISNSLA |
| | CDR1 | |
| 52 | anti-BsgD LC | DASSLQV |
| | CDR2 | |
| 53 | anti-BsgD LC | QQGYKYPFT |
| | CDR3 | |
| 54 | anti-BsgD HC | GFTLSNYYM |
| | CDR1 | |
| 55 | anti-BsgD HC | SISTGGGYTYYRDSVKG |
| | CDR2 | |
| 56 | anti-BsgD HC | ARSLINYRNYGDYVMDA |
| | CDR3 | |
| 57 | anti-BsgD LC | DIQMTQSPASLSASLGETVSIEC |
| | FR1 | |
| 58 | anti-BsgD LC | WYQQKPGKSPQLLIY |
| | FR2 | |
| 59 | anti-BsgD LC | GVPSRFSGSGSGTQYSLKISGLQPEDEGVYYC |
| | FR3 | |
| 60 | anti-BsgD LC | FGSGTKLEIK |
| | FR4 | |
| 61 | anti-BsgD HC | EVQLVESGGGLVQPGRSMKLSCAAS |
| | FR1 | |
| 62 | anti-BsgD HC | WVRQAPTKGLEWVA |
| | FR2 | |
| 63 | anti-BsgD HC | RFTISRDLAKSTLYLQMDSLRSEDTATYHC |
| | FR3 | |
| 64 | anti-BsgD HC | WGQGASVTVSS |
| | FR4 | |
| 65 | anti-BsgE | QFTLTQPKSVSGSLRSTITIPCERSSGDIGHNYVSWYQQHL |
| | Light chain | GRPPINVIYADDQRPSEVSDRFSGSIDSSSNSASLTITNLQM |
| | variable | DDEADYFCQSYDSNVDIVFGGGTKLTVL |
| | domain | |
| | polypeptide | |
| 66 | anti-BsgE | QVQLKESGPGLVQPSQTLSLTCSVSGLSLTTSSLSWIRQPP |
| | heavy chain | GKGLEWMGGIWSKGGTEYNSPIKSRLSISRDTSKSQIFLKM |
| | variable | NSLQTEDTAMYFCARNGVYHNYWYFDFWGPGTMVTVSS |
| | domain | |
| | polypeptide | |
| 67 | anti-BsgE LC | ERSSGDIGHNYVS |
| | CDR1 | |
| 68 | anti-BsgE LC | ADDQRPS |
| | CDR2 | |
| 69 | anti-BsgE LC | QSYDSNVDIV |
| | CDR3 | |
| 70 | anti-BsgE HC | GLSLTTSSLS |
| | CDR1 | |
| 71 | anti-BsgE HC | GIWSKGGTEYNSPIKS |
| | CDR2 | |
| 72 | anti-BsgE HC | ARNGVYHNYWYFDF |
| | CDR3 | |
| 73 | anti-BsgE LC | QFTLTQPKSVSGSLRSTITIPC |
| | FR1 | |
| 74 | anti-BsgE LC | WYQQHLGRPPINVIY |
| | FR2 | |
| 75 | anti-BsgE LC | EVSDRFSGSIDSSSNSASLTITNLQMDDEADYFC |
| | FR3 | |
| 76 | anti-BsgE LC | FGGGTKLTVL |
| | FR4 | |
| 77 | anti-BsgE HC | QVQLKESGPGLVQPSQTLSLTCSVS |
| | FR1 | |
| 78 | anti-BsgE HC | WIRQPPGKGLEWMG |
| | FR2 | |
| 79 | anti-BsgE HC | RLSISRDTSKSQIFLKMNSLQTEDTAMYFC |
| | FR3 | |
| 80 | anti-BsgE HC | WGPGTMVTVSS |
| | FR4 | |
| 81 | anti-Glut1 | DIVLTQSPSSLSASLGDTITITCHASQNINVWLSWYQQKPG |
| | Light chain | NIPKLLIYKASNLHSGVPSRFSGSGSGTGFTLTISSLQPEDIA |
| | variable | TYYCQQGQTFPYTFGGGTRLEIK |
| | domain | |
| | polypeptide | |
| 82 | anti-Glut1 | QVQLQQPGSVLVRPGASVKLSCKASGYTFTGSWLHWAK |
| | heavy chain | QRPGQGLEWIGEIHPYSGNTNYNERFKGKATLTVDTPSST |
| | variable | AYVDLRSLTFEDSAVYYCAKEGGWFLRIYGMDYWGQGT |
| | domain | SVTVSS |
| | polypeptide | |
| 83 | anti-Glut1 LC | HASQNINVWLS |
| | CDR1 | |
| 84 | anti-Glut1 LC | KASNLHS |
| | CDR2 | |
| 85 | anti-Glut1 LC | QQGQTFPYT |
| | CDR3 | |
| 86 | anti-Glut1 HC | GYTFTGSWLH |
| | CDR1 | |
| 87 | anti-Glut1 HC | EIHPYSGNTNYNERFKG |
| | CDR2 | |
| 88 | anti-Glut1 HC | AKEGGWFLRIYGMDY |
| | CDR3 | |
| 89 | anti-Glut1 LC | DIVLTQSPSSLSASLGDTITITC |
| | FR1 | |
| 90 | anti-Glut1 LC | WYQQKPGNIPKLLIY |
| | FR2 | |
| 91 | anti-Glut1 LC | GVPSRFSGSGSGTGFTLTISSLQPEDIATYYC |
| | FR3 | |
| 92 | anti-Glut1 LC | FGGGTRLEIK |
| | FR4 | |
| 93 | anti-Glut1 HC | QVQLQQPGSVLVRPGASVKLSCKAS |
| | FR1 | |
| 94 | anti-Glut1 HC | WAKQRPGQGLEWIG |
| | FR2 | |
| 95 | anti-Glut1 HC | KATLTVDTPSSTAYVDLRSLTFEDSAVYYC |
| | FR3 | |
| 96 | anti-Glut1 HC | WGQGTSVTVSS |
| | FR4 | |
| 97 | Human | MELQPPEASI AVVSIPRQLP GSHSEAGVQG LSAGDDSETG |
| | CD98hc | SDCVTQAGLQ LLASSDPPAL ASKNAEVTVE |
| | isoform b | TGFHHVSQAD IEFLTSIDPT ASASGSAGITGTMSQDTEVD |
| | polypeptide | MKEVELNELE PEKQPMNAAS GAAMSLAGAE |
| | | KNGLVKIKVA EDEAEAAAAA KFTGLSKEEL |
| | | LKVAGSPGWV WLGWLGMLAG AVVIIVRAPR |
| | | CRELPAQKWW HTGALYRIGD LQAFQGHGAG |
| | | NLAGLKGRLD YLSSLKVKGL VLGPIHKNQK |
| | | DDVAQTDLLQ IDPNFGSKED FDSLLQSAKK KSIRVILDLT |
| | | PNYRGENSWF STQVDTVATK VKDALEFWLQ |
| | | AGVDGFQVRD IENLKDASSF LAEWQNITKG |
| | | FSEDRLLIAG TNSSDLQQIL SLLESNKDLL LTSSYLSDSG |
| | | STGEHTKSLV TQYLNATGNR WCSWSLSQAR |
| | | LLTSFLPAQL LRLYQLMLFT LPGTPVFSYG DEIGLDAAAL |
| | | PGQPMEAPVM LWDESSFPDI PGAVSANMTV |
| | | KGQSEDPGSL LSLFRRLSDQ RSKERSLLHG |
| | | DFHAFSAGPG LFSYIRHWDQ NERFLVVLNF |
| | | GDVGLSAGLQ ASDLPASASL PAKADLLLST |
| | | QPGREEGSPL ELERLKLEPH EGLLLRFPYA A |
| 98 | Human | agttccagggaaggagggcgtagacaaagcgccacctgaacttgcggcgcgaaaaaggc |
| | CD98hc | gcgcatgcgtcctacgggagcgtgctggctcaccgaccgcattgcggcttggttttctcacc |
| | isoform b | cagtgcatgtggcaggagcggtgagatcactgcctcacggcgatcctggactgacggtcac |
| | polynucleotide | gactgcctaccctctaaccctgttctgagctgccccttgcccacacaccccaaacctgtgtgc |
| | | aggatccgcctccatggagctacagcctcctgaagcctcgatcgccgtcgtgtcgattccgc |
| | | gccagttgcctggctcacattcggaggctggtgtccagggtctcagcgcgggggacgactc |
| | | agagacggggtctgactgtgttacccaggctggtcttcaactcttggcctcaagtgatcctcct |
| | | gccttagcttccaagaatgctgaggttacagtagaaacggggtttcaccatgttagccaggct |
| | | gatattgaattcctgacctcaattgatccgactgcctcggcctccggaagtgctgggattaca |
| | | ggcaccatgagccaggacaccgaggtggatatgaaggaggtggagctgaatgagttaga |
| | | gcccgagaagcagccgatgaacgcggcgtctggggcggccatgtccctggcgggagcc |
| | | gagaagaatggtctggtgaagatcaaggtggcggaagacgaggcggaggcggcagccg |
| | | cggctaagttcacgggcctgtccaaggaggagctgctgaaggtggcaggcagccccggct |
| | | gggtacgcacccgctgggcactgctgctgctcttctggctcggctggctcggcatgcttgct |
| | | ggtgccgtggtcataatcgtgcgagcgccgcgttgtcgcgagctaccggcgcagaagtggt |
| | | ggcacacgggcgccctctaccgcatcggcgaccttcaggccttccagggccacggcgcg |
| | | ggcaacctggcgggtctgaaggggcgtctcgattacctgagctctctgaaggtgaagggcc |
| | | ttgtgctgggtccaattcacaagaaccagaaggatgatgtcgctcagactgacttgctgcaga |
| | | tcgaccccaattttggctccaaggaagattttgacagtctcttgcaatcggctaaaaaaaaga |
| | | gcatccgtgtcattctggaccttactcccaactaccggggtgagaactcgtggttctccactca |
| | | ggttgacactgtggccaccaaggtgaaggatgctctggagttttggctgcaagctggcgtgg |
| | | atgggttccaggttcgggacatagagaatctgaaggatgcatcctcattcttggctgagtggc |
| | | aaaatatcaccaagggcttcagtgaagacaggctcttgattgcggggactaactcctccgac |
| | | cttcagcagatcctgagcctactcgaatccaacaaagacttgctgttgactagctcatacctgt |
| | | ctgattctggttctactggggagcatacaaaatccctagtcacacagtatttgaatgccactgg |
| | | caatcgctggtgcagctggagtttgtctcaggcaaggctcctgacttccttcttgccggctcaa |
| | | cttctccgactctaccagctgatgctcttcaccctgccagggacccctgttttcagctacgggg |
| | | atgagattggcctggatgcagctgcccttcctggacagcctatggaggctccagtcatgctgt |
| | | gggatgagtccagcttccctgacatcccaggggctgtaagtgccaacatgactgtgaaggg |
| | | ccagagtgaagaccctggctccctcctttccttgttccggcggctgagtgaccagcggagta |
| | | aggagcgctccctactgcatggggacttccacgcgttctccgctgggcctggactcttctcct |
| | | atatccgccactgggaccagaatgagcgttttctggtagtgcttaactttggggatgtgggcct |
| | | ctcggctggactgcaggcctccgacctgcctgccagcgccagcctgccagccaaggctga |
| | | cctcctgctcagcacccagccaggccgtgaggagggctcccctcttgagctggaacgcctg |
| | | aaactggagcctcacgaagggctgctgctccgcttcccctacgcggcctgacttcagcctga |
| | | catggacccactacccttctcctttccttcccaggccctttggcttctgatttttctcttttttaaaaa |
| | | caaacaaacaaactgttgcagattatgagtgaacccccaaatagggtgttttctgccttcaaat |
| | | aaaagtcacccctgcatggtgaagtcttccctctgcttctctcataaaaaaa |
| 99 | Human | MELQPPEASI AVVSIPRQLP GSHSEAGVQG LSAGDDSELG |
| | CD98hc | SHCVAQTGLE LLASGDPLPS ASQNAEMIET |
| | isoform c | GSDCVTQAGL QLLASSDPPA LASKNAEVTG |
| | polypeptide | TMSQDTEVDM KEVELNELEP EKQPMNAASG |
| | | AAMSLAGAEK NGLVKIKVAE DEAEAAAAAK |
| | | FTGLSKEELL KVAGSPGWVR TRWALLLLFW |
| | | LGWLGMLAGA VVIIVRAPRC RELPAQKWWH |
| | | TGALYRIGDL QAFQGHGAGN LAGLKGRLDY |
| | | LSSLKVKGLV LGPIHKNQKD DVAQTDLLQI |
| | | DPNFGSKEDF DSLLQSAKKK SIRVILDLTP NYRGENSWFS |
| | | TQVDTVATKV KDALEFWLQA GVDGFQVRDI |
| | | ENLKDASSFL AEWQNITKGF SEDRLLIAGT NSSDLQQILS |
| | | LLESNKDLLL TSSYLSDSGS TGEHTKSLVT |
| | | QYLNATGNRW CSWSLSQARL LTSFLPAQLL |
| | | RLYQLMLFTL PGTPVFSYGD EIGLDAAALP |
| | | GQPMEAPVML WDESSFPDIP GAVSANMTVK |
| | | GQSEDPGSLL SLFRRLSDQR SKERSLLHGD FHAFSAGPGL |
| | | FSYIRHWDQN ERFLVVLNFG DVGLSAGLQA |
| | | SDLPASASLP AKADLLLSTQ PGREEGSPLE LERLKLEPHE |
| | | GLLLRFPYAA |
| 100 | Human | agttccagggaaggagggcgtagacaaagcgccacctgaacttgcggcgcgaaaaaggc |
| | CD98hc | gcgcatgcgtcctacgggagcgtgctggctcaccgaccgcattgcggcttggttttctcacc |
| | isoform c | cagtgcatgtggcaggagcggtgagatcactgcctcacggcgatcctggactgacggtcac |
| | polynucleotide | gactgcctaccctctaaccctgttctgagctgccccttgcccacacaccccaaacctgtgtgc |
| | | aggatccgcctccatggagctacagcctcctgaagcctcgatcgccgtcgtgtcgattccgc |
| | | gccagttgcctggctcacattcggaggctggtgtccagggtctcagcgcgggggacgactc |
| | | agagttggggtctcactgtgttgcccagactggtctcgaactcttggcctcaggtgatcctctt |
| | | ccctcagcttcccagaatgccgagatgatagagacggggtctgactgtgttacccaggctgg |
| | | tcttcaactcttggcctcaagtgatcctcctgccttagcttccaagaatgctgaggttacaggca |
| | | ccatgagccaggacaccgaggtggatatgaaggaggtggagctgaatgagttagagcccg |
| | | agaagcagccgatgaacgcggcgtctggggcggccatgtccctggcgggagccgagaa |
| | | gaatggtctggtgaagatcaaggtggcggaagacgaggcggaggcggcagccgcggct |
| | | aagttcacgggcctgtccaaggaggagctgctgaaggtggcaggcagccccggctgggt |
| | | acgcacccgctgggcactgctgctgctcttctggctcggctggctcggcatgcttgctggtg |
| | | ccgtggtcataatcgtgcgagcgccgcgttgtcgcgagctaccggcgcagaagtggtggc |
| | | acacgggcgccctctaccgcatcggcgaccttcaggccttccagggccacggcgcgggc |
| | | aacctggcgggtctgaaggggcgtctcgattacctgagctctctgaaggtgaagggccttgt |
| | | gctgggtccaattcacaagaaccagaaggatgatgtcgctcagactgacttgctgcagatcg |
| | | accccaattttggctccaaggaagattttgacagtctcttgcaatcggctaaaaaaaagagcat |
| | | ccgtgtcattctggaccttactcccaactaccggggtgagaactcgtggttctccactcaggtt |
| | | gacactgtggccaccaaggtgaaggatgctctggagttttggctgcaagctggcgtggatg |
| | | ggttccaggttcgggacatagagaatctgaaggatgcatcctcattcttggctgagtggcaaa |
| | | atatcaccaagggcttcagtgaagacaggctcttgattgcggggactaactcctccgaccttc |
| | | agcagatcctgagcctactcgaatccaacaaagacttgctgttgactagctcatacctgtctga |
| | | ttctggttctactggggagcatacaaaatccctagtcacacagtatttgaatgccactggcaat |
| | | cgctggtgcagctggagtttgtctcaggcaaggctcctgacttccttcttgccggctcaacttc |
| | | tccgactctaccagctgatgctcttcaccctgccagggacccctgttttcagctacggggatg |
| | | agattggcctggatgcagctgcccttcctggacagcctatggaggctccagtcatgctgtgg |
| | | gatgagtccagcttccctgacatcccaggggctgtaagtgccaacatgactgtgaagggcc |
| | | agagtgaagaccctggctccctcctttccttgttccggcggctgagtgaccagcggagtaag |
| | | gagcgctccctactgcatggggacttccacgcgttctccgctgggcctggactcttctcctat |
| | | atccgccactgggaccagaatgagcgttttctggtagtgcttaactttggggatgtgggcctct |
| | | cggctggactgcaggcctccgacctgcctgccagcgccagcctgccagccaaggctgac |
| | | ctcctgctcagcacccagccaggccgtgaggagggctcccctcttgagctggaacgcctga |
| | | aactggagcctcacgaagggctgctgctccgcttcccctacgcggcctgacttcagcctgac |
| | | atggacccactacccttctcctttccttcccaggccctttggcttctgatttttctcttttttaaaaac |
| | | aaacaaacaaactgttgcagattatgagtgaacccccaaatagggtgttttctgccttcaaata |
| | | aaagtcacccctgcatggtgaagtcttccctctgcttctctcataaaaaaa |
| 101 | Human | MELQPPEASI AVVSIPRQLP GSHSEAGVQG |
| | CD98hc | LSAGDDSGTM SQDTEVDMKE VELNELEPEK |
| | isoform e | QPMNAASGAA MSLAGAEKNG LVKIKVAEDE |
| | polypeptide | AEAAAAAKFT GLSKEELLKV AGSPGWVRTR |
| | | WALLLLFWLG WLGMLAGAVV IIVRAPRCRE |
| | | LPAQKWWHTG ALYRIGDLQA FQGHGAGNLA |
| | | GLKGRLDYLS SLKVKGLVLG PIHKNQKDDV |
| | | AQTDLLQIDP NFGSKEDFDS LLQSAKKKSI RVILDLTPNY |
| | | RGENSWFSTQ VDTVATKVKD ALEFWLQAGV |
| | | DGFQVRDIEN LKDASSFLAE WQNITKGFSE DRLLIAGTNS |
| | | SDLQQILSLL ESNKDLLLTS SYLSDSGSTG EHTKSLVTQY |
| | | LNATGNRWCS WSLSQARLLT SFLPAQLLRL |
| | | YQLMLFTLPG TPVFSYGDEI GLDAAALPGQ |
| | | PMEAPVMLWD ESSFPDIPGA VSANMTVKGQ |
| | | SEDPGSLLSL FRRLSDQRSK ERSLLHGDFH AFSAGPGLFS |
| | | YIRHWDQNER FLVVLNFGDV GLSAGLQASD |
| | | LPASASLPAK ADLLLSTQPG REEGSPLELE RLKLEPHEGL |
| | | LLRFPYAA |
| 102 | Human | agttccagggaaggagggcgtagacaaagcgccacctgaacttgcggcgcgaaaaaggc |
| | CD98hc | gcgcatgcgtcctacgggagcgtgctggctcaccgaccgcattgcggcttggttttctcacc |
| | isoform e | cagtgcatgtggcaggagcggtgagatcactgcctcacggcgatcctggactgacggtcac |
| | polynucleotide | gactgcctaccctctaaccctgttctgagctgccccttgcccacacaccccaaacctgtgtgc |
| | | aggatccgcctccatggagctacagcctcctgaagcctcgatcgccgtcgtgtcgattccgc |
| | | gccagttgcctggctcacattcggaggctggtgtccagggtctcagcgcgggggacgactc |
| | | aggcaccatgagccaggacaccgaggtggatatgaaggaggtggagctgaatgagttaga |
| | | gcccgagaagcagccgatgaacgcggcgtctggggcggccatgtccctggcgggagcc |
| | | gagaagaatggtctggtgaagatcaaggtggcggaagacgaggcggaggcggcagccg |
| | | cggctaagttcacgggcctgtccaaggaggagctgctgaaggtggcaggcagccccggct |
| | | gggtacgcacccgctgggcactgctgctgctcttctggctcggctggctcggcatgcttgct |
| | | ggtgccgtggtcataatcgtgcgagcgccgcgttgtcgcgagctaccggcgcagaagtggt |
| | | ggcacacgggcgccctctaccgcatcggcgaccttcaggccttccagggccacggcgcg |
| | | ggcaacctggcgggtctgaaggggcgtctcgattacctgagctctctgaaggtgaagggcc |
| | | ttgtgctgggtccaattcacaagaaccagaaggatgatgtcgctcagactgacttgctgcaga |
| | | tcgaccccaattttggctccaaggaagattttgacagtctcttgcaatcggctaaaaaaaaga |
| | | gcatccgtgtcattctggaccttactcccaactaccggggtgagaactcgtggttctccactca |
| | | ggttgacactgtggccaccaaggtgaaggatgctctggagttttggctgcaagctggcgtgg |
| | | atgggttccaggttcgggacatagagaatctgaaggatgcatcctcattcttggctgagtggc |
| | | aaaatatcaccaagggcttcagtgaagacaggctcttgattgcggggactaactcctccgac |
| | | cttcagcagatcctgagcctactcgaatccaacaaagacttgctgttgactagctcatacctgt |
| | | ctgattctggttctactggggagcatacaaaatccctagtcacacagtatttgaatgccactgg |
| | | caatcgctggtgcagctggagtttgtctcaggcaaggctcctgacttccttcttgccggctcaa |
| | | cttctccgactctaccagctgatgctcttcaccctgccagggacccctgttttcagctacgggg |
| | | atgagattggcctggatgcagctgcccttcctggacagcctatggaggctccagtcatgctgt |
| | | gggatgagtccagcttccctgacatcccaggggctgtaagtgccaacatgactgtgaaggg |
| | | ccagagtgaagaccctggctccctcctttccttgttccggcggctgagtgaccagcggagta |
| | | aggagcgctccctactgcatggggacttccacgcgttctccgctgggcctggactcttctcct |
| | | atatccgccactgggaccagaatgagcgttttctggtagtgcttaactttggggatgtgggcct |
| | | ctcggctggactgcaggcctccgacctgcctgccagcgccagcctgccagccaaggctga |
| | | cctcctgctcagcacccagccaggccgtgaggagggctcccctcttgagctggaacgcctg |
| | | aaactggagcctcacgaagggctgctgctccgcttcccctacgcggcctgacttcagcctga |
| | | catggacccactacccttctcctttccttcccaggccctttggcttctgatttttctcttttttaaaaa |
| | | caaacaaacaaactgttgcagattatgagtgaacccccaaatagggtgttttctgccttcaaat |
| | | aaaagtcacccctgcatggtgaagtcttccctctgcttctctcataaaaaaa |
| 103 | Human | MSQDTEVDMK EVELNELEPE KQPMNAASGA |
| | CD98hc | AMSLAGAEKN GLVKIKVAED EAEAAAAAKF |
| | isoform f | TGLSKEELLK VAGSPGWVRT RWALLLLFWL |
| | polypeptide | GWLGMLAGAV VIIVRAPRCR ELPAQKWWHT |
| | | GALYRIGDLQ AFQGHGAGNL AGLKGRLDYL |
| | | SSLKVKGLVL GPIHKNQKDD VAQTDLLQID |
| | | PNFGSKEDFD SLLQSAKKKS IRVILDLTPN YRGENSWFST |
| | | QVDTVATKVK DALEFWLQAG VDGFQVRDIE |
| | | NLKDASSFLA EWQNITKGFS EDRLLIAGTN SSDLQQILSL |
| | | LESNKDLLLT SSYLSDSGST GEHTKSLVTQ |
| | | YLNATGNRWC SWSLSQARLL TSFLPAQLLR |
| | | LYQLMLFTLP GTPVFSYGDE IGLDAAALPG |
| | | QPMEAPVMLW DESSFPDIPG AVSANMTVKG |
| | | QSEDPGSLLS LFRRLSDQRS KERSLLHGDF HAFSAGPGLF |
| | | SYIRHWDQNE RFLVVLNFGD VGLSAGLQAS |
| | | DLPASASLPA KADLLLSTQP GREEGSPLEL ERLKLEPHEG |
| | | LLLRFPYAA |
| 104 | Human | cagaggccgcgcctgctgctgagcagatgcagtagccgaaactgcgcggaggcacagag |
| | CD98hc | gccggggagagcgttctgggtccgagggtccaggtaggggttgagccaccatctgaccgc |
| | isoform f | aagctgcgtcgtgtcgccggttctgcaggcaccatgagccaggacaccgaggtggatatga |
| | polynucleotide | aggaggtggagctgaatgagttagagcccgagaagcagccgatgaacgcggcgtctggg |
| | | gcggccatgtccctggcgggagccgagaagaatggtctggtgaagatcaaggtggcgga |
| | | agacgaggcggaggcggcagccgcggctaagttcacgggcctgtccaaggaggagctg |
| | | ctgaaggtggcaggcagccccggctgggtacgcacccgctgggcactgctgctgctcttct |
| | | ggctcggctggctcggcatgcttgctggtgccgtggtcataatcgtgcgagcgccgcgttgt |
| | | cgcgagctaccggcgcagaagtggtggcacacgggcgccctctaccgcatcggcgacctt |
| | | caggccttccagggccacggcgcgggcaacctggcgggtctgaaggggcgtctcgattac |
| | | ctgagctctctgaaggtgaagggccttgtgctgggtccaattcacaagaaccagaaggatga |
| | | tgtcgctcagactgacttgctgcagatcgaccccaattttggctccaaggaagattttgacagt |
| | | ctcttgcaatcggctaaaaaaaagagcatccgtgtcattctggaccttactcccaactaccgg |
| | | ggtgagaactcgtggttctccactcaggttgacactgtggccaccaaggtgaaggatgctct |
| | | ggagttttggctgcaagctggcgtggatgggttccaggttcgggacatagagaatctgaagg |
| | | atgcatcctcattcttggctgagtggcaaaatatcaccaagggcttcagtgaagacaggctctt |
| | | gattgcggggactaactcctccgaccttcagcagatcctgagcctactcgaatccaacaaag |
| | | acttgctgttgactagctcatacctgtctgattctggttctactggggagcatacaaaatcccta |
| | | gtcacacagtatttgaatgccactggcaatcgctggtgcagctggagtttgtctcaggcaagg |
| | | ctcctgacttccttcttgccggctcaacttctccgactctaccagctgatgctcttcaccctgcc |
| | | agggacccctgttttcagctacggggatgagattggcctggatgcagctgcccttcctggac |
| | | agcctatggaggctccagtcatgctgtgggatgagtccagcttccctgacatcccaggggct |
| | | gtaagtgccaacatgactgtgaagggccagagtgaagaccctggctccctcctttccttgttc |
| | | cggcggctgagtgaccagcggagtaaggagcgctccctactgcatggggacttccacgcg |
| | | ttctccgctgggcctggactcttctcctatatccgccactgggaccagaatgagcgttttctggt |
| | | agtgcttaactttggggatgtgggcctctcggctggactgcaggcctccgacctgcctgcca |
| | | gcgccagcctgccagccaaggctgacctcctgctcagcacccagccaggccgtgaggag |
| | | ggctcccctcttgagctggaacgcctgaaactggagcctcacgaagggctgctgctccgctt |
| | | cccctacgcggcctgacttcagcctgacatggacccactacccttctcctttccttcccaggc |
| | | cctttggcttctgatttttctcttttttaaaaacaaacaaacaaactgttgcagattatgagtgaac |
| | | ccccaaatagggtgttttctgccttcaaataaaagtcacccctgcatggtgaagtcttccctctg |
| | | cttctctcataaaaaaa |
| 105 | Murine | MDPEPTEHST DGVSVPRQPP SAQTGLDVQV |
| | CD98hc | VSAAGDSGTM SQDTEVDMKD VELNELEPEK |
| | isoform a | QPMNAADGAA AGEKNGLVKI KVAEDETEAG |
| | polypeptide | VKFTGLSKEE LLKVAGSPGW VRTRWALLLL |
| | | FWLGWLGMLA GAVVIIVRAP RCRELPVQRW |
| | | WHKGALYRIG DLQAFVGRDA GGIAGLKSHL |
| | | EYLSTLKVKG LVLGPIHKNQ KDEINETDLK QINPTLGSQE |
| | | DFKDLLQSAK KKSIHIILDL TPNYQGQNAW |
| | | FLPAQADIVA TKMKEALSSW LQDGVDGFQF |
| | | RDVGKLMNAP LYLAEWQNIT KNLSEDRLLI |
| | | AGTESSDLQQ IVNILESTSD LLLTSSYLSN STFTGERTES |
| | | LVTRFLNATG SQWCSWSVSQ AGLLADFIPD |
| | | HLLRLYQLLL FTLPGTPVFS YGDELGLQGA |
| | | LPGQPAKAPL MPWNESSIFH IPRPVSLNMT |
| | | VKGQNEDPGS LLTQFRRLSD LRGKERSLLH |
| | | GDFHALSSSP DLFSYIRHWD QNERYLVVLN |
| | | FRDSGRSARL GASNLPAGIS LPASAKLLLS TDSARQSREE |
| | | DTSLKLENLS LNPYEGLLLQ FPFVA |
| 106 | Murine | gtgggtagaggaatccgcccaaaggggcgtgcggagagctccgcctctgattttgcagcg |
| | CD98hc | cgaaaaagaggcgcaggcgctttaggggagtgcgacgctacgcctttggcgctgcggcta |
| | isoform a | ggcggttcttactcactgcgggtaaaacgtcatcgctggagattttggttcgcgacccataca |
| | polynucleotide | gctcgactgtctgggtcacaactaccaatatccatacgttgaggcgatttctcaccctcactca |
| | | cgctaagccgcgtgttgatccatctctatggatcctgaacctactgaacactccaccgacggt |
| | | gtctcggttccccgccagccgcccagcgcgcagacggggcttgatgtccaggttgtcagcg |
| | | cagcgggcgactcaggcaccatgagccaggacaccgaagtggacatgaaagatgtggag |
| | | ctgaacgagctagaaccggagaagcagcccatgaatgcagcggacggggcggcggccg |
| | | gggagaagaacggtctggtgaagatcaaggtggcggaggacgagacggaggccggggt |
| | | caagttcaccggcttatccaaggaggagctactgaaggtagcgggcagccctggctgggt |
| | | gcgcacccgctgggcgctgctgctgctcttctggctcggttggctgggcatgctggcgggc |
| | | gccgtggttatcatcgttcgggcgccgcgctgccgtgagctgcctgtacagaggtggtggc |
| | | acaagggcgccctctaccgcatcggcgaccttcaggcctttgtaggccgggatgcgggag |
| | | gcatagctggtctgaagagccatctggagtacttgagcaccctgaaggtgaagggcctggt |
| | | gttaggcccaattcacaagaaccagaaggatgaaatcaatgaaaccgacctgaaacagatt |
| | | aatcccactttgggctcccaggaagattttaaagaccttctacaaagtgccaagaaaaagag |
| | | cattcacatcattttggacctcactcccaactaccagggccagaatgcgtggttcctccctgct |
| | | caggctgacattgtagccaccaaaatgaaggaagctctgagttcttggttgcaggacggtgt |
| | | ggatggtttccaattccgggatgtgggaaagctgatgaatgcacccttgtacttggctgagtg |
| | | gcagaatatcaccaagaacttaagtgaggacaggcttttgattgcagggactgagtcctctga |
| | | cctgcagcaaattgtcaacatacttgaatccaccagcgacctgctgttgaccagctcctacct |
| | | gtcaaattccactttcactggggagcgtactgaatccctagtcactaggtttttgaatgccactg |
| | | gcagccaatggtgcagctggagtgtgtcgcaagcaggactcctcgcagactttataccgga |
| | | ccatcttctccgactctaccagctgctgctcttcactctgccagggactcctgtttttagctacg |
| | | gggatgagcttggccttcagggtgcccttcctggacagcctgcgaaggccccactcatgcc |
| | | gtggaatgagtccagcatctttcacatcccaagacctgtaagcctcaacatgacagtgaagg |
| | | gccagaatgaagaccctggctccctccttacccagttccggcggctgagtgaccttcggggt |
| | | aaggagcgctctctgttgcacggtgacttccatgcactgtcttcctcacctgacctcttctccta |
| | | catacgacactgggaccagaatgagcgttacctggtggtgctcaacttccgagattcgggcc |
| | | ggtcagccaggctaggggcctccaacctccctgctggcataagcctgccagccagcgcta |
| | | aacttttgcttagtaccgacagtgcccggcaaagccgtgaggaggacacctccctgaagct |
| | | ggaaaacctgagcctgaatccttatgagggcttgctgttacagttcccctttgtggcctgatcct |
| | | tcctatgcagaacctaccaccctcctttgttctccccaggccttttggattctagtcttcctctcct |
| | | tgtttttaaacttttgcagattacatacgaattcttatactgggtgtttttgtcttcaaataaaaacat |
| | | cacccctgcctcatgagattgtgactttcatccttccttccttctagaagaactttctcttgctcct |
| | | gatctcttttgctcctccctgcccctgccatagtcgcagccagttgtagacagctattccagctc |
| | | tctttttttttttttttttttttttttttttggtttttcgagacagggtttctctgtatagccctggctgtcctg |
| | | gaactcactttgtagaccaggctggcctcgaactcagaaatccacctgcctctgcctcccaa |
| | | gtgctgggattaaaggcgtgcgccaccacgcccggccgctattccagctcttaaattaatcat |
| | | ttagagaccaaggctagagaagggcccttccatggttaacagcaaagtgtcttggctggagt |
| | | aaccacacctcctcgctctggcccaagaatcttgggaattgccaactcttccttatctctcttag |
| | | cacagtctttaagaaaaagggtggggtgagttgaagactgcatactgccaagggcctgggg |
| | | cttcccttctttactctttggtgaggcacttaccatatagacaggactgcgatccccagtaccca |
| | | gtggataccccatctccagaaaaagccaacaagacaaaccctttgcttccttaggctatgttat |
| | | ctcttgtgtggaaatggagaagaaataaggaataaacattttttgtatgaag |
| 107 | Murine | MSQDTEVDMK DVELNELEPE KQPMNAADGA |
| | CD98hc | AAGEKNGLVK IKVAEDETEA GVKFTGLSKE |
| | isoform b | ELLKVAGSPG WVRTRWALLL LFWLGWLGML |
| | polypeptide | AGAVVIIVRA PRCRELPVQR WWHKGALYRI |
| | | GDLQAFVGRD AGGIAGLKSH LEYLSTLKVK |
| | | GLVLGPIHKN QKDEINETDL KQINPTLGSQ EDFKDLLQSA |
| | | KKKSIHIILD LTPNYQGQNA WFLPAQADIV |
| | | ATKMKEALSS WLQDGVDGFQ FRDVGKLMNA |
| | | PLYLAEWQNI TKNLSEDRLL IAGTESSDLQ QIVNILESTS |
| | | DLLLTSSYLS NSTFTGERTE SLVTRFLNAT |
| | | GSQWCSWSVS QAGLLADFIP DHLLRLYQLL |
| | | LFTLPGTPVF SYGDELGLQG ALPGQPAKAP |
| | | LMPWNESSIF HIPRPVSLNM TVKGQNEDPG SLLTQFRRLS |
| | | DLRGKERSLL HGDFHALSSS PDLFSYIRHW |
| | | DQNERYLVVL NFRDSGRSAR LGASNLPAGI |
| | | SLPASAKLLL STDSARQSRE EDTSLKLENL SLNPYEGLLL |
| | | QFPFVA |
| 108 | Murine | cccgccgccacacccgcccagcggcagaagcagttaggaagctctgctagcctcacggc |
| | CD98hc | cacgggacgcctctctgaacggggatccaggcaggattagagctgcctcactgactacag |
| | isoform b | gccgtgtcgtgtcaccgtttctgcaggcaccatgagccaggacaccgaagtggacatgaaa |
| | polynucleotide | gatgtggagctgaacgagctagaaccggagaagcagcccatgaatgcagcggacgggg |
| | | cggcggccggggagaagaacggtctggtgaagatcaaggtggcggaggacgagacgg |
| | | aggccggggtcaagttcaccggcttatccaaggaggagctactgaaggtagcgggcagcc |
| | | ctggctgggtgcgcacccgctgggcgctgctgctgctcttctggctcggttggctgggcatg |
| | | ctggcgggcgccgtggttatcatcgttcgggcgccgcgctgccgtgagctgcctgtacaga |
| | | ggtggtggcacaagggcgccctctaccgcatcggcgaccttcaggcctttgtaggccggg |
| | | atgcgggaggcatagctggtctgaagagccatctggagtacttgagcaccctgaaggtgaa |
| | | gggcctggtgttaggcccaattcacaagaaccagaaggatgaaatcaatgaaaccgacctg |
| | | aaacagattaatcccactttgggctcccaggaagattttaaagaccttctacaaagtgccaag |
| | | aaaaagagcattcacatcattttggacctcactcccaactaccagggccagaatgcgtggttc |
| | | ctccctgctcaggctgacattgtagccaccaaaatgaaggaagctctgagttcttggttgcag |
| | | gacggtgtggatggtttccaattccgggatgtgggaaagctgatgaatgcacccttgtacttg |
| | | gctgagtggcagaatatcaccaagaacttaagtgaggacaggcttttgattgcagggactga |
| | | gtcctctgacctgcagcaaattgtcaacatacttgaatccaccagcgacctgctgttgaccag |
| | | ctcctacctgtcaaattccactttcactggggagcgtactgaatccctagtcactaggtttttga |
| | | atgccactggcagccaatggtgcagctggagtgtgtcgcaagcaggactcctcgcagacttt |
| | | ataccggaccatcttctccgactctaccagctgctgctcttcactctgccagggactcctgttttt |
| | | agctacggggatgagcttggccttcagggtgcccttcctggacagcctgcgaaggccccac |
| | | tcatgccgtggaatgagtccagcatctttcacatcccaagacctgtaagcctcaacatgacag |
| | | tgaagggccagaatgaagaccctggctccctccttacccagttccggcggctgagtgacctt |
| | | cggggtaaggagcgctctctgttgcacggtgacttccatgcactgtatcctcacctgacctct |
| | | tctcctacatacgacactgggaccagaatgagcgttacctggtggtgctcaacttccgagatt |
| | | cgggccggtcagccaggctaggggcctccaacctccctgctggcataagcctgccagcca |
| | | gcgctaaacttttgcttagtaccgacagtgcccggcaaagccgtgaggaggacacctccct |
| | | gaagctggaaaacctgagcctgaatccttatgagggcttgctgttacagttcccctttgtggcc |
| | | tgatccttcctatgcagaacctaccaccctcctttgttctccccaggccttttggattctagtcttc |
| | | ctctccttgtttttaaacttttgcagattacatacgaattcttatactgggtgtttttgtcttcaaataa |
| | | aaacatcacccctgcctcatgagattgtgactttcatccttccttccttctagaagaactttctctt |
| | | gctcctgatctcttttgctcctccctgcccctgccatagtcgcagccagttgtagacagctattc |
| | | cagctctctttttttttttttttttttttttttttttggtttttcgagacagggtttctctgtatagccctggc |
| | | tgtcctggaactcactttgtagaccaggctggcctcgaactcagaaatccacctgcctctgcc |
| | | tcccaagtgctgggattaaaggcgtgcgccaccacgcccggccgctattccagctcttaaat |
| | | taatcatttagagaccaaggctagagaagggcccttccatggttaacagcaaagtgtcttggc |
| | | tggagtaaccacacctcctcgctctggcccaagaatcttgggaattgccaactcttccttatct |
| | | ctcttagcacagtctttaagaaaaagggtggggtgagttgaagactgcatactgccaagggc |
| | | ctggggcttcccttctttactctttggtgaggcacttaccatatagacaggactgcgatcccca |
| | | gtacccagtggataccccatctccagaaaaagccaacaagacaaaccctttgcttccttagg |
| | | ctatgttatctcttgtgtggaaatggagaagaaataaggaataaacattttttgtatgaag |
| 109 | Macaca | MSQDTEVDMK EVELNELEPE KQPMNAASGA |
| | fascicularis | AMAVVGAEKN GLVKIKVAED EAEAAAAAKF |
| | CD98hc | TGLSKEELLK VAGSPGWVRT RWVLLLLFWL |
| | polypeptide | GWLGMLAGAV VIIVRAPRCR ELPAQKWWHT |
| | | GALYRIGDLQ AFQGHGSGNL AGLKGRLDYL |
| | | SSLKVKGLVL GPLHKNQKDD VAQTDLLQID |
| | | PNFGSKEDFD NLLQSAKKKS IRVILDLTPN YRGENLWFST |
| | | QVDSVATKVK DALEFWLQAG VDGFQVRDIE |
| | | NLKDASSFLA EWENITKGFS EDRLLIAGTN SSDLQQIVSP |
| | | LESNKDLLLT SSYLSDSSFT GEHTKSLVTQ |
| | | YLNATGNRWC SWSLSQAGLL TSFLPAQLLR |
| | | LYQLMLSTLP GTPVFSYGDE IGLKAAALPG |
| | | QPVEAPVMLW DESSFPDIPG AVSANMTVKG |
| | | QSEDPGSLLS LFRQLSDQRS KERSLLHGDF HTFSSGPGLF |
| | | SYIRHWDQNE RFLVVLNFGD VGLSAGLQAS |
| | | DLPASASLPT KADPVLSTQP GREEGSPLEL ERLKLEPHEG |
| | | LLLRFPYVA |
| 110 | Macaca | agatgcagtagccgaagctgcgcggaggcacacaggccgggagaccgttctgggtccga |
| | fascicularis | gggtccgggcaggggttgagccaccatctgacctcaagcttcgtcgtgtcgccggttctgca |
| | CD98hc | ggcaccatgagccaggacaccgaggtggatatgaaggaggtggagctgaatgagttagaa |
| | polynucleotide | cccgagaagcagccgatgaacgcggcgtctggggctgccatggccgtggtgggagccga |
| | | gaagaatggtctggtgaagatcaaggtggcggaagacgaggcggaggcagcagccgcc |
| | | gctaagttcacgggcctgtccaaggaggagctgctgaaggtggcgggcagtcccggctgg |
| | | gtacgtacccgctgggtgctgctgctgctcttctggctcggctggcttggcatgctggcgggt |
| | | gccgtggtcataatcgtgcgggcgccgcgctgtcgcgagctgccggcgcagaagtggtgg |
| | | cacacgggcgccctctaccgcatcggcgaccttcaggccttccagggccacggctcgggc |
| | | aacttggcgggtctgaaggggcgtctcgattacctgagctctctgaaggtgaagggccttgt |
| | | gctgggcccacttcacaagaaccagaaggacgatgtcgctcagaccgacttgctgcagatc |
| | | gaccccaattttggctccaaggaagattttgacaatctcttgcaatcggctaaaaaaaagagc |
| | | atccgtgtcattctggacctcactcccaactaccggggtgagaacttgtggttctccacccag |
| | | gttgacagtgtggccaccaaggtgaaggatgctctggagttttggctgcaagctggcgtgga |
| | | tgggttccaggttcgggacatagagaatctgaaggatgcatcctcattcttggctgagtggga |
| | | aaacatcaccaagggcttcagtgaagataggctcttgattgcagggactaactcctccgacct |
| | | tcagcagatcgtgagcccactcgaatccaacaaagacttgctgttgaccagctcatacctgtc |
| | | tgattccagctttactggggagcatacaaaatccctagtcacacagtatttgaatgccactggc |
| | | aatcgctggtgcagctggagtttgtctcaggcagggctcctgacttccttcttgccggctcaac |
| | | ttctccgactctaccagctgatgctctccaccctgccagggacccctgtgttcagctacgggg |
| | | atgagattggcctgaaggcagctgcccttcctggacagcctgtggaggctccagtcatgctg |
| | | tgggatgagtccagcttccctgacatcccaggggctgtaagtgccaacatgactgtgaagg |
| | | gccagagtgaagaccctggctccctcctttccttgttccggcagctgagtgaccagcggagt |
| | | aaggagcgctccctattgcatggggacttccatacgttctcctctgggcctggactcttctcct |
| | | atatccgccactgggaccagaatgagcgttttctggtagtgcttaactttggggatgtgggcct |
| | | ctcggctgggctgcaggcctccgacctgcccgccagcgccagcctgccaaccaaggctg |
| | | accctgtgctcagcacccagccaggccgtgaggagggctccccgcttgagctggaacgcc |
| | | tgaaactggagcctcacgaagggctgctgctccgcttcccctatgtggcctgaccccagcct |
| | | gacgtggacccactgccctcctttccttcctagaccctttgggttctggtttttctctttttccccct |
| | | tttttaaaaaacaacaacaaaacggttgcagattataaatgaacccccaaatagggtgttttctg |
| | | ccttcaaataaaagtcacccctgcctggtgaaaaaaaaaaaaaaaaaaaaaaaaaaaaaaa |
| | | aa |
| 111 | BACE1 | MAQALPWLLLWMGAGVLPAHGTQHGIRLPLRSGLGGAP |
| | polypeptide | LGLRLPRETDEEPEEPGRRGSFVEMVDNLRGKSGQGYYVE |
| | | MTVGSPPQTLNILVDTGSSNFAVGAAPHPFLHRYYQRQLS |
| | | STYRDLRKGVYVPYTQGKWEGELGTDLVSIPHGPNVTVR |
| | | ANIAAITESDKFFINGSNWEGILGLAYAEIARPDDSLEPFFD |
| | | SLVKQTHVPNLFSLQLCGAGFPLNQSEVLASVGGSMIIGGI |
| | | DHSLYTGSLWYTPIRREWYYEVIIVRVEINGQDLKMDCKE |
| | | YNYDKSIVDSGTTNLRLPKKVFEAAVKSIKAASSTEKFPD |
| | | GFWLGEQLVCWQAGTTPWNIFPVISLYLMGEVTNQSFRIT |
| | | ILPQQYLRPVEDVATSQDDCYKFAISQSSTGTVMGAVIME |
| | | GFYVVFDRARKRIGFAVSACHVHDEFRTAAVEGPFVTLD |
| | | MEDCGYNIPQTDESTLMTIAYVMAAICALFMLPLCLMVC |
| | | QWCCLRCLRQQHDDFADDISLLK |
| 112 | Human | MAAALFVLLG FALLGTHGAS GAAGTVFTTV |
| | basigin | EDLGSKILLT CSLNDSATEV TGHRWLKGGV |
| | isoform 2 | VLKEDALPGQ KTEFKVDSDD QWGEYSCVFL |
| | polypeptide | PEPMGTANIQ LHGPPRVKAV KSSEHINEGE |
| | | TAMLVCKSES VPPVTDWAWY KITDSEDKAL |
| | | MNGSESRFFV SSSQGRSELH IENLNMEADP |
| | | GQYRCNGTSS KGSDQAIITL RVRSVLVLVT IIFIYEKRRK |
| | | PEDVLDDDDA GSAPLKSSGQ HQNDKGKNVR QRNSS |
| 113 | Murine | MAAALLLALA FTLLSGQGAC AAAGTIQTSV |
| | basigin | QEVNSKTQLT CSLNSSGVDI VGHRWMRGGK |
| | polypeptide | VLQEDTLPDL HTKYIVDADD RSGEYSCIFL PEPVGRSEIN |
| | | VEGPPRIKVG KKSEHSSEGE LAKLVCKSDA |
| | | SYPPITDWFW FKTSDTGEEE AITNSTEANG |
| | | KYVVVSTPEK SQLTISNLDV NVDPGTYVCN |
| | | ATNAQGTTRE TISLRVRSRG NSRAQVTDKK IEPRGPTIKP |
| | | CPPCKCPAPN LLGGPSVFIF PPKIKDVLMI SLSPIVTCVV |
| | | VDVSEDDPDV QISWFVNNVE VHTAQTQTHR |
| | | EDYNSTLRVV SALPIQHQDW MSGKEFKCKV |
| | | NNKDLPAPIE RTISKPKGSV RAPQVYVLPP |
| | | PEEEMTKKQV TLTCMVTDFM PEDIYVEWTN |
| | | NGKTELNYKN TEPVLDSDGS YFMYSKLRVE |
| | | KKNWVERNSY SCSVVHEGLH NHHTTKSFSR TPGK |
| 114 | Human Glut1 | MEPSSKKLTG RLMLAVGGAV LGSLQFGYNT |
| | polypeptide | GVINAPQKVI EEFYNQTWVH RYGESILPTT |
| | | LTTLWSLSVA IFSVGGMIGS FSVGLFVNRF |
| | | GRRNSMLMMN LLAFVSAVLM GFSKLGKSFE |
| | | MLILGRFIIG VYCGLTTGFV PMYVGEVSPT |
| | | ALRGALGTLH QLGIVVGILI AQVFGLDSIM |
| | | GNKDLWPLLL SIIFIPALLQ CIVLPFCPES PRFLLINRNE |
| | | ENRAKSVLKK LRGTADVTHD LQEMKEESRQ |
| | | MMREKKVTIL ELFRSPAYRQ PILIAVVLQL SQQLSGINAV |
| | | FYYSTSIFEK AGVQQPVYAT IGSGIVNTAF TVVSLFVVER |
| | | AGRRTLHLIG LAGMAGCAIL MTIALALLEQ |
| | | LPWMSYLSIV AIFGFVAFFE VGPGPIPWFI VAELFSQGPR |
| | | PAAIAVAGFS NWTSNFIVGM CFQYVEQLCG PYVFIIFTVL |
| | | LVLFFIFTYF KVPETKGRTF DEIASGFRQG GASQSDKTPE |
| | | ELFHPLGADS QV |

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entireties by reference.

1. A method of transporting an agent across the blood-brain barrier, wherein the method comprises exposing the blood-brain barrier to an antibody which (i) binds to a blood-brain barrier receptor (BBB-R); and (ii) is coupled to the agent; wherein:
the antibody, upon binding to the BBB-R, transports the agent coupled thereto across the blood-brain barrier; and
the BBB-R is a member selected from the group consisting of CD98 heavy chain (CD98hc), basigin, and Glucose Transporter Type 1 (Glut1).

2. The method of claim 1, wherein the blood-brain barrier is in a mammal.

3. The method of claim 2, wherein the mammal has a neurological disease or disorder.

4. A method of treating a neurological disease or disorder in a mammal, wherein the method comprises administering to the mammal an antibody which (i) binds to a BBB-R selected from the group consisting of CD98hc, basigin, and Glut1; and (ii) is coupled to a therapeutic agent which is effective for treating the neurological disease or disorder.

5. The method of claim 4, wherein the neurological disease or disorder is selected from the group consisting of Alzheimer's disease (AD), stroke, dementia, muscular dystrophy (MD), multiple sclerosis (MS), amyotrophic lateral sclerosis (ALS), cystic fibrosis, Angelman's syndrome, Liddle syndrome, Parkinson's disease, Pick's disease, Paget's disease, cancer, and traumatic brain injury.

6. The method of claim 2, wherein the mammal is a human.

7. The method of claim 1, wherein the agent is an imaging agent.

8. The method of claim 1, wherein the agent is a neurological disorder drug.

9. The method of claim 1, wherein binding of the antibody to the BBB-R does not impair binding of the BBB-R to one or more of its native ligands.

10. The method of claim 1, wherein binding of the BBB-R to one or more of its native ligands in the presence of the antibody is at least 80% of the amount of binding in the absence of the antibody.

11. The method of claim 1, wherein binding of the antibody to the BBB-R does not impair transport of one or more of the native ligands of the BBB-R across the blood-brain barrier.

12. The method of claim 1, wherein transport of one or more of the native ligands of the BBB-R across the blood-brain barrier is at least 80% of the amount of transport in the absence of the antibody.

13. The method of claim 1, wherein the antibody has been engineered to have a low binding affinity.

14. The method of claim 1, wherein the antibody does not inhibit cell proliferation and/or cell division and/or cell adhesion.

15. The method of claim 1, wherein the antibody does not induce cell death.

16. The method of claim 1, wherein the antibody has an IC₅₀ for the BBB-R from about 1 nM to about 100 μM.

17. The method of claim 16, wherein the IC₅₀ is from about 1 nM to about 10 nM.

18. The method of claim 17, wherein the IC₅₀ is from about 5 nM to about 100 μM.

19. The method of claim 18, wherein the IC₅₀ is from about 50 nM to about 100 μM.

20. The method of claim 16, wherein the IC₅₀ is from about 100 nM to about 100 μM.

21. The method of claim 1, wherein the antibody has an affinity for the BBB-R from about 1 nM to about 10 μM.

22. The method of claim 21, wherein the antibody has an affinity for the BBB-R from about 1 nM to about 1 μM.

23. The method of claim 22, wherein the antibody has an affinity for the BBB-R from about 1 nM to about 500 nM.

24. The method of claim 23, wherein the antibody has an affinity for the BBB-R from about 1 nM to about 50 nM.

25. The method of claim 1, wherein the antibody has an affinity for the BBB-R from about 1 nM to about 100 μM.

26. The method of claim 1, wherein the antibody is administered to the mammal at a therapeutic dose.

27. The method of claim 26, wherein the therapeutic dose is BBB-R-saturating.

28. The method of claim 1, wherein the antibody is multispecific, and the agent coupled thereto comprises an antigen-binding site of the multispecific antibody which binds to a brain antigen.

29. The method of claim 28, wherein the multispecific antibody is bispecific.

30. The method of claim 28, wherein the brain antigen is selected from the group consisting of: beta-secretase 1 (BACE1), Abeta, epidermal growth factor receptor (EGFR), human epidermal growth factor receptor 2 (HER2), Tau, apolipoprotein (e.g., apolipoprotein E4 (ApoE4)), alpha-synuclein, CD20, huntingtin, prion protein (PrP), leucine rich repeat kinase 2 (LRRK2), parkin, presenilin 1, presenilin 2, gamma secretase, death receptor 6 (DR6), amyloid precursor protein (APP), p75 neurotrophin receptor (p75NTR), and caspase 6.

31. The method of claim 28, wherein the multispecific antibody binds both CD98hc and BACEI.

32. The method of claim 28, wherein the multispecific antibody binds both CD98hc and Abeta.

33. The method of claim 28, wherein the multispecific antibody binds both basigin and BACEI.

34. The method of claim 28, wherein the multispecific antibody binds both basigin and Abeta.

35. The method of claim 28, wherein the multispecific antibody binds both Glut1 and BACEI.

36. The method of claim 28, wherein the multispecific antibody binds both Glut1 and Abeta.

37. The method of claim 1, wherein the BBB-R is CD98hc.

38. The method of claim 1, wherein the BBB-R is basigin.

39. The method of claim 38, wherein the antibody comprises:
(a) one or more of the heavy chain complementarity determining region (CDR) 1, 2, and 3 sequences comprising the amino acid sequences of SEQ ID NOs: 6, 7, and 8, respectively; and/or
(b) one or more of the light chain CDR 1, 2, and 3 sequences comprising the amino acid sequences of SEQ ID NOs: 3, 4, and 5, respectively.

40. The method of claim 39, wherein the antibody further comprises:
(a) a light chain variable domain framework FR1 amino acid sequence of SEQ ID NO: 9;
(b) a light chain variable domain framework FR2 amino acid sequence of SEQ ID NO: 10;
(c) a light chain variable domain framework FR3 amino acid sequence of SEQ ID NO: 11; and/or
(d) a light chain variable domain framework FR4 amino acid sequence of SEQ ID NO: 12.

41. The method of claim 39, wherein the antibody further comprises:
(a) a heavy chain variable domain framework FR1 amino acid sequence of SEQ ID NO: 13;
(b) a heavy chain variable domain framework FR2 amino acid sequence of SEQ ID NO: 14;
(c) a heavy chain variable domain framework FR3 amino acid sequence of SEQ ID NO: 15; and/or
(d) a heavy chain variable domain framework FR4 amino acid sequence of SEQ ID NO: 16.

42. The method of claim 38, wherein the antibody comprises:
(a) a heavy chain variable region (VH) sequence having at least 95% sequence identity to the amino acid sequence of SEQ ID NO: 2; (b) a light chain variable region (VL) sequence having at least 95% sequence identity to the amino acid sequence of SEQ ID NO: 1; or (c) a VH sequence as in (a) and a VL sequence as in (b).

43. The method of claim 38, wherein the antibody comprises a VH sequence of SEQ ID NO: 2 and a VL sequence of SEQ ID NO: 1.

44. The method of claim 38, wherein the antibody comprises:
(a) one or more of the heavy chain CDR 1, 2, and 3 sequences comprising the amino acid sequences of SEQ ID NOs: 22, 23, and 24, respectively; and/or
(b) one or more of the light chain CDR 1, 2, and 3 sequences comprising the amino acid sequences of SEQ ID NOs: 19, 20, and 21, respectively.

45. The method of claim 44, wherein the antibody further comprises:
(a) a light chain variable domain framework FR1 amino acid sequence of SEQ ID NO: 25;
(b) a light chain variable domain framework FR2 amino acid sequence of SEQ ID NO: 26;
(c) a light chain variable domain framework FR3 amino acid sequence of SEQ ID NO: 27; and/or
(d) a light chain variable domain framework FR4 amino acid sequence of SEQ ID NO: 28.

46. The method of claim 44, wherein the antibody further comprises:
(a) a heavy chain variable domain framework FR1 amino acid sequence of SEQ ID NO: 29;
(b) a heavy chain variable domain framework FR2 amino acid sequence of SEQ ID NO: 30;
(c) a heavy chain variable domain framework FR3 amino acid sequence of SEQ ID NO: 31; and/or
(d) a heavy chain variable domain framework FR4 amino acid sequence of SEQ ID NO: 32.

47. The method of claim 38, wherein the antibody comprises: (a) VH sequence having at least 95% sequence identity to the amino acid sequence of SEQ ID NO: 18; (b) VL sequence having at least 95% sequence identity to the amino acid sequence of SEQ ID NO: 17; or (c) a VH sequence as in (a) and a VL sequence as in (b).

48. The method of claim 47, wherein the antibody comprises a VH sequence of SEQ ID NO: 18 and a VL sequence of SEQ ID NO: 17.

49. The method of claim 38, wherein the antibody comprises:
(a) one or more of the heavy chain CDR 1, 2, and 3 sequences comprising the amino acid sequences of SEQ ID NOs: 38, 39, and 40, respectively; and/or
(b) one or more of the light chain CDR 1, 2, and 3 sequences comprising the amino acid sequences of SEQ ID NOs: 35, 36, and 37, respectively.

50. The method of claim 49, wherein the antibody further comprises:
(a) a light chain variable domain framework FR1 amino acid sequence of SEQ ID NO: 41;
(b) a light chain variable domain framework FR2 amino acid sequence of SEQ ID NO: 42;
(c) a light chain variable domain framework FR3 amino acid sequence of SEQ ID NO: 43; and/or
(d) a light chain variable domain framework FR4 amino acid sequence of SEQ ID NO: 44.

51. The method of claim 49, wherein the antibody further comprises: (a) a heavy chain variable domain framework FR1 amino acid sequence of SEQ ID NO: 45; (b) a heavy chain variable domain framework FR2 amino acid sequence of SEQ ID NO: 46; (c) a heavy chain variable domain framework FR3 amino acid sequence of SEQ ID NO: 47; and/or (d) a heavy chain variable domain framework FR4 amino acid sequence of SEQ ID NO: 48.

52. The method of claim 38, wherein the antibody comprises:
(a) VH sequence having at least 95% sequence identity to the amino acid sequence of SEQ ID NO: 34; or (b) VL sequence having at least 95% sequence identity to the amino acid sequence of SEQ ID NO: 33; or (c) a VH sequence as in (a) and a VL sequence as in (b).

53. The method of claim 38, wherein the antibody comprises a VH sequence of SEQ ID NO: 34 and a VL sequence of SEQ ID NO: 33.

54. The method of claim 38, wherein the antibody comprises:
(a) one or more of the heavy chain CDR 1, 2, and 3 sequences comprising the amino acid sequences of SEQ ID NOs: 54, 55, and 56, respectively; and/or
(b) one or more of the light chain CDR 1, 2, and 3 sequences comprising the amino acid sequences of SEQ ID NOs: 51, 52, and 53, respectively.

55. The method of claim 54, wherein the antibody further comprises: (a) a light chain variable domain framework FR1 amino acid sequence of SEQ ID NO: 57; (b) a light chain variable domain framework FR2 amino acid sequence of SEQ ID NO: 58; (c) a light chain variable domain framework FR3 amino acid sequence of SEQ ID NO: 59; and/or (d) a light chain variable domain framework FR4 amino acid sequence of SEQ ID NO: 60.

56. The method of claim 54, wherein the antibody further comprises:
(a) a heavy chain variable domain framework FR1 amino acid sequence of SEQ ID NO: 61;
(b) a heavy chain variable domain framework FR2 amino acid sequence of SEQ ID NO: 62;
(c) a heavy chain variable domain framework FR3 amino acid sequence of SEQ ID NO: 63; and/or
(d) a heavy chain variable domain framework FR4 amino acid sequence of SEQ ID NO: 64.

57. The method of claim 38, wherein the antibody comprises:
(a) VH sequence having at least 95% sequence identity to the amino acid sequence of SEQ ID NO: 50; or (b) VL sequence having at least 95% sequence identity to the amino acid sequence of SEQ ID NO: 49; or (c) a VH sequence as in (a) and a VL sequence as in (b).

58. The method of claim 38, wherein the antibody comprises a VH sequence of SEQ ID NO: 50 and a VL sequence of SEQ ID NO: 49.

59. The method of claim 38, wherein the antibody comprises:
(a) one or more of the heavy chain CDR 1, 2, and 3 sequences comprising the amino acid sequences of SEQ ID NOs: 70, 71, and 72, respectively; and/or
(b) one or more of the light chain CDR 1, 2, and 3 sequences comprising the amino acid sequences of SEQ ID NOs: 67, 68, and 69, respectively.

60. The method of claim 59, wherein the antibody further comprises:
(a) a light chain variable domain framework FR1 amino acid sequence of SEQ ID NO: 73;
(b) a light chain variable domain framework FR2 amino acid sequence of SEQ ID NO: 74;
(c) a light chain variable domain framework FR3 amino acid sequence of SEQ ID NO: 75; and/or
(d) a light chain variable domain framework FR4 amino acid sequence of SEQ ID NO: 76.

61. The method of claim 59, wherein the antibody further comprises:
(a) a heavy chain variable domain framework FR1 amino acid sequence of SEQ ID NO: 77;
(b) a heavy chain variable domain framework FR2 amino acid sequence of SEQ ID NO: 78;
(c) a heavy chain variable domain framework FR3 amino acid sequence of SEQ ID NO: 79; and/or
(d) a heavy chain variable domain framework FR4 amino acid sequence of SEQ ID NO: 80.

62. The method of claim 38, wherein the antibody comprises:
(a) VH sequence having at least 95% sequence identity to the amino acid sequence of SEQ ID NO: 66; or (b) VL sequence having at least 95% sequence identity to the amino acid sequence of SEQ ID NO: 65; or (c) a VH sequence as in (a) and a VL sequence as in (b).

63. The method of claim 38, wherein the antibody comprises a VH sequence of SEQ ID NO: 66 and a VL sequence of SEQ ID NO: 65.

64. The method of claim 1, wherein the BBB-R is Glut1.

65. The method of claim 64, wherein the antibody comprises:
(a) one or more of the heavy chain CDR 1, 2, and 3 sequences comprising the amino acid sequences of SEQ ID NOs: 86, 87 and 88, respectively; and/or
(b) one or more of the light chain CDR 1, 2, and 3 sequences comprising the amino acid sequences of SEQ ID NOs: 83, 84 and 85, respectively.

66. The method of claim 64, wherein the antibody comprises:
(a) a light chain variable domain framework FR1 amino acid sequence of SEQ ID NO: 89;
(b) a light chain variable domain framework FR2 amino acid sequence of SEQ ID NO: 90;
(c) a light chain variable domain framework FR3 amino acid sequence of SEQ ID NO: 91; and/or
(d) a light chain variable domain framework FR4 amino acid sequence of SEQ ID NO: 92.

67. The method of claim 64, wherein the antibody comprises:
(a) a heavy chain variable domain framework FR1 amino acid sequence of SEQ ID NO: 93;
(b) a heavy chain variable domain framework FR2 amino acid sequence of SEQ ID NO: 94;
(c) a heavy chain variable domain framework FR3 amino acid sequence of SEQ ID NO: 95; and/or
(d) a heavy chain variable domain framework FR4 amino acid sequence of SEQ ID NO: 96.

68. The method of claim 64, wherein the antibody comprises:
(a) a VH sequence having at least 95% sequence identity to the amino acid sequence of SEQ ID NO: 82; (b) a VL sequence having at least 95% sequence identity to the amino acid sequence of SEQ ID NO: 81; or (c) a VH sequence as in (a) and a VL sequence as in (b).

69. The method of claim 64, wherein the antibody comprises a VH sequence of SEQ ID NO: 82 and a VL sequence of SEQ ID NO: 81.

70. An isolated antibody that binds to basigin, wherein the antibody comprises:
(a) one or more of the heavy chain complementarity determining region (CDR) 1, 2, and 3 sequences comprising the amino acid sequences of SEQ ID NOs: 6, 7, and 8, respectively; and/or
(b) one or more of the light chain CDR 1, 2, and 3 sequences comprising the amino acid sequences of SEQ ID NOs: 3, 4, and 5, respectively.

71. The antibody of claim 70, further comprising one or more of:
(a) a light chain variable domain framework FR1 amino acid sequence of SEQ ID NO: 9;
(b) a light chain variable domain framework FR2 amino acid sequence of SEQ ID NO: 10;
(c) a light chain variable domain framework FR3 amino acid sequence of SEQ ID NO: 11; and
(d) a light chain variable domain framework FR4 amino acid sequence of SEQ ID NO: 12.

72. The antibody of claim 70, further comprising one or more of:
(a) a heavy chain variable domain framework FR1 amino acid sequence of SEQ ID NO: 13;
(b) a heavy chain variable domain framework FR2 amino acid sequence of SEQ ID NO: 14;
(c) a heavy chain variable domain framework FR3 amino acid sequence of SEQ ID NO: 15; and
(d) a heavy chain variable domain framework FR4 amino acid sequence of SEQ ID NO: 16.

73. The antibody of claim 70, comprising:
(a) a VH sequence having at least 95% sequence identity to the amino acid sequence of SEQ ID NO: 2; (b) a VL sequence having at least 95% sequence identity to the amino acid sequence of SEQ ID NO: 1; or (c) a VH sequence as in (a) and a VL sequence as in (b).

74. The antibody of claim 70, wherein the antibody comprises a VH sequence of SEQ ID NO: 2 and a VL sequence of SEQ ID NO: 1.

75. An isolated antibody that binds to basigin, wherein the antibody comprises:
(a) one or more of heavy chain CDR 1, 2, and 3 sequences comprising the amino acid sequences of SEQ ID NOs: 22, 23, and 24, respectively; and/or
(b) one or more of light chain CDR 1, 2, and 3 sequences comprising the amino acid sequences of SEQ ID NOs: 19, 20, and 21, respectively.

76. The antibody of claim 75, further comprising one or more of:
(a) a light chain variable domain framework FR1 amino acid sequence of SEQ ID NO: 25;
(b) a light chain variable domain framework FR2 amino acid sequence of SEQ ID NO: 26;
(c) a light chain variable domain framework FR3 amino acid sequence of SEQ ID NO: 27; and
(d) a light chain variable domain framework FR4 amino acid sequence of SEQ ID NO: 28.

77. The antibody of claim 75, further comprising one or more of:
(a) a heavy chain variable domain framework FR1 amino acid sequence of SEQ ID NO: 29;
(b) a heavy chain variable domain framework FR2 amino acid sequence of SEQ ID NO: 30;
(c) a heavy chain variable domain framework FR3 amino acid sequence of SEQ ID NO: 31; and
(d) a heavy chain variable domain framework FR4 amino acid sequence of SEQ ID NO: 32.

78. The antibody of claim 75, comprising:
(a) a VH sequence having at least 95% sequence identity to the amino acid sequence of SEQ ID NO: 18; (b) a VL sequence having at least 95% sequence identity to the amino acid sequence of SEQ ID NO: 17; or (c) a VH sequence as in (a) and a VL sequence as in (b).

79. The antibody of claim 75, wherein the antibody comprises a VH sequence of SEQ ID NO: 18 and a VL sequence of SEQ ID NO: 17.

80. An isolated antibody that binds to basigin, wherein the antibody comprises:
(a) one or more of the heavy chain CDR 1, 2, and 3 sequences comprising the amino acid sequences of SEQ ID NOs: 38, 39, and 40, respectively; and/or
(b) one or more of the light chain CDR 1, 2, and 3 sequences comprising the amino acid sequences of SEQ ID NOs: 35, 36, and 37, respectively.

81. The antibody of claim 80, wherein the antibody further comprises:
(a) a light chain variable domain framework FR1 amino acid sequence of SEQ ID NO: 41;
(b) a light chain variable domain framework FR2 amino acid sequence of SEQ ID NO: 42;
(c) a light chain variable domain framework FR3 amino acid sequence of SEQ ID NO: 43; and/or
(d) a light chain variable domain framework FR4 amino acid sequence of SEQ ID NO: 44.

82. The antibody of claim 80, wherein the antibody further comprises:
(a) a heavy chain variable domain framework FR1 amino acid sequence of SEQ ID NO: 45;
(b) a heavy chain variable domain framework FR2 amino acid sequence of SEQ ID NO: 46;
(c) a heavy chain variable domain framework FR3 amino acid sequence of SEQ ID NO: 47; and/or
(d) a heavy chain variable domain framework FR4 amino acid sequence of SEQ ID NO: 48.

83. The antibody of claim 80, wherein the antibody comprises:
(a) VH sequence having at least 95% sequence identity to the amino acid sequence of SEQ ID NO: 34; or (b) VL sequence having at least 95% sequence identity to the amino acid sequence of SEQ ID NO: 33; or (c) a VH sequence as in (a) and a VL sequence as in (b).

84. The antibody of claim 80, wherein the antibody comprises a VH sequence of SEQ ID NO: 34 and a VL sequence of SEQ ID NO: 33.

85. An isolated antibody that binds to basigin, wherein the antibody comprises:
(a) one or more of the heavy chain CDR 1, 2, and 3 sequences comprising the amino acid sequences of SEQ ID NOs: 54, 55, and 56, respectively; and/or
(b) one or more of the light chain CDR 1, 2, and 3 sequences comprising the amino acid sequences of SEQ ID NOs: 51, 52, and 53, respectively.

86. The antibody of claim 85, wherein the antibody further comprises:
(a) a light chain variable domain framework FR1 amino acid sequence of SEQ ID NO: 57;
(b) a light chain variable domain framework FR2 amino acid sequence of SEQ ID NO: 58;
(c) a light chain variable domain framework FR3 amino acid sequence of SEQ ID NO: 59; and/or
(d) a light chain variable domain framework FR4 amino acid sequence of SEQ ID NO: 60.

87. The antibody of claim 85, wherein the antibody further comprises:
(a) a heavy chain variable domain framework FR1 amino acid sequence of SEQ ID NO: 61; (b) a heavy chain variable domain framework FR2 amino acid sequence of SEQ ID NO: 62;
(c) a heavy chain variable domain framework FR3 amino acid sequence of SEQ ID NO: 63; and/or
(d) a heavy chain variable domain framework FR4 amino acid sequence of SEQ ID NO: 64.

88. The antibody of claim 85, wherein the antibody comprises:
(a) VH sequence having at least 95% sequence identity to the amino acid sequence of SEQ ID NO: 50; or (b) VL sequence having at least 95% sequence identity to the amino acid sequence of SEQ ID NO: 49; or (c) a VH sequence as in (a) and a VL sequence as in (b).

89. The antibody of claim 85, wherein the antibody comprises a VH sequence of SEQ ID NO: 50 and a VL sequence of SEQ ID NO: 49.

90. An isolated antibody that binds to basigin, wherein the antibody comprises:
(a) one or more of the heavy chain CDR 1, 2, and 3 sequences comprising the amino acid sequences of SEQ ID NOs: 70, 71, and 72, respectively; and/or
(b) one or more of the light chain CDR 1, 2, and 3 sequences comprising the amino acid sequences of SEQ ID NOs: 67, 68, and 69, respectively.

91. The antibody of claim 90, wherein the antibody further comprises:
(a) a light chain variable domain framework FR1 amino acid sequence of SEQ ID NO: 73;
(b) a light chain variable domain framework FR2 amino acid sequence of SEQ ID NO: 74;
(c) a light chain variable domain framework FR3 amino acid sequence of SEQ ID NO: 75; and/or
(d) a light chain variable domain framework FR4 amino acid sequence of SEQ ID NO: 76.

92. The antibody of claim 90, wherein the antibody further comprises:
(a) a heavy chain variable domain framework FR1 amino acid sequence of SEQ ID NO: 77;
(b) a heavy chain variable domain framework FR2 amino acid sequence of SEQ ID NO: 78;
(c) a heavy chain variable domain framework FR3 amino acid sequence of SEQ ID NO: 79; and/or
(d) a heavy chain variable domain framework FR4 amino acid sequence of SEQ ID NO: 80.

93. The antibody of claim 90, wherein the antibody comprises:
(a) VH sequence having at least 95% sequence identity to the amino acid sequence of SEQ ID NO: 66; or (b) VL sequence having at least 95% sequence identity to the amino acid sequence of SEQ ID NO: 65; or (c) a VH sequence as in (a) and a VL sequence as in (b).

94. The antibody of claim 90, wherein the antibody comprises a VH sequence of SEQ ID NO: 66 and a VL sequence of SEQ ID NO: 65.

95. An isolated antibody that binds to Glut1, wherein the antibody comprises:
(a) one or more of the heavy chain CDR 1, 2, and 3 sequences comprising the amino acid sequences of SEQ ID NOs: 86, 87, and 88, respectively; and/or
(b) one or more of the light chain CDR 1, 2, and 3 sequences comprising the amino acid sequences of SEQ ID NOs: 83, 84, and 85, respectively.

96. The antibody of claim 95, further comprising one or more of:
(a) a light chain variable domain framework FR1 amino acid sequence of SEQ ID NO: 89;
(b) a light chain variable domain framework FR2 amino acid sequence of SEQ ID NO: 90;
(c) a light chain variable domain framework FR3 amino acid sequence of SEQ ID NO: 91; and
(d) a light chain variable domain framework FR4 amino acid sequence of SEQ ID NO: 92.

97. The antibody of claim 95, further comprising one or more of:
(a) a heavy chain variable domain framework FR1 amino acid sequence of SEQ ID NO: 93;
(b) a heavy chain variable domain framework FR2 amino acid sequence of SEQ ID NO: 94;
(c) a heavy chain variable domain framework FR3 amino acid sequence of SEQ ID NO: 95; and
(d) a heavy chain variable domain framework FR4 amino acid sequence of SEQ ID NO: 96.

98. The antibody of claim 95, comprising:
(a) a VH sequence having at least 95% sequence identity to the amino acid sequence of SEQ ID NO: 82; (b) a VL sequence having at least 95% sequence identity to the amino acid sequence of SEQ ID NO: 81; or (c) a VH sequence as in (a) and a VL sequence as in (b).

99. The antibody of claim 95, wherein the antibody comprises a VH sequence of SEQ ID NO: 82 and a VL sequence of SEQ ID NO: 81.

100. The antibody of claim 1, which is a monoclonal antibody.

101. The antibody of claim 1, which is a human, humanized, or chimeric antibody.

102. The antibody of claim 1, which is a full length IgG1 or IgG4 antibody.

103. The antibody of claim 1, which is a Fab fragment.

104. An isolated nucleic acid encoding the antibody of claim 1.

105. A host cell comprising the nucleic acid of claim 104.

106. A method of producing an antibody comprising culturing the host cell of claim 105 so that the antibody is produced.

107. An immunoconjugate comprising the antibody of claim 70, and a cytotoxic agent.

108. A multispecific antibody comprising a first arm which comprises an antigen-binding site of the antibody of claim 70.

109. The multispecific antibody of claim 108, further comprising a second arm which comprises an antigen binding site which binds a brain antigen.

110. The multispecific antibody of claim 109, wherein the brain antigen is selected from the group consisting of: BACE1, Abeta, EGFR, HER2, Tau, apolipoprotein (e.g., ApoE4), alpha-synuclein, CD20, huntingtin, PrP, LRRK2, parkin, presenilin 1, presenilin 2, gamma secretase, DR6, APP, p75NTR, and caspase 6.

111. The multispecific antibody of claim 110 wherein the brain antigen is BACE1.

112. The multispecific antibody of claim 110 wherein the brain antigen is Abeta.

113. A pharmaceutical formulation comprising the antibody of claim 70, and a pharmaceutically acceptable carrier.

114. The pharmaceutical formulation of claim 113, further comprising an additional therapeutic agent.

115. (canceled)

116. (canceled)

117. The antibody of claim 70 for use in transporting an agent across the blood-brain barrier, wherein the use comprises: exposing the blood-brain barrier to the antibody.

118. (canceled)

119. (canceled)

120. (canceled)

121. The antibody of claim 70, wherein the BBB-R is a human BBB-R.

122. The antibody of claim 70, which is coupled with a neurological disorder drug.
